(19) United States
(12) Patent Application Publication
(10) Pub. No.: US 2012/0016006 A1
(43) Pub. Date: Jan. 19, 2012

(54) Compositions And Methods For Increasing Cellular Uptake Of RNAi Via SID-1

(75) Inventors: Gregory Hinkle, Plymouth, MA (US); Yosef Landesman, Cambridge, MA (US)

(73) Assignee: Alnylam Pharmaceuticals ,Inc. a corporation

(21) Appl. No.: 13/128,835

(22) PCT Filed: Nov. 17, 2009
(86) PCT No.: PCT/US2009/064866

§ 371 Date: Aug. 5, 2011

Related U.S. Application Data
(60) Provisional application No. 61/115,130, filed on Nov. 17, 2008.

(51) Int. Cl. A61K 31/713; C12N 5/071; C12N 5/10; C12N 15/113; C12N 15/63

(52) U.S. Cl. 514/44 A; 536/24.5; 435/320.1; 435/325; 435/357; 435/354; 435/353; 435/352; 435/350; 435/363; 435/375

(57) ABSTRACT

The invention relates to a double-stranded ribonucleic acid (dsRNA) targeting a Systemic RNA Interference Defective-1 (SID-I) gene, and methods of using the dsRNA to inhibit expression of SID-1.

### RELATED APPLICATIONS

This application claims the benefit of the earlier-filed U.S. Provisional Application No. 61/115,130, filed on Nov. 17, 2008, hereby incorporated-by-reference.

### FIELD OF THE INVENTION

The invention relates to a double-stranded ribonucleic acid (dsRNA) targeting a Systemic RNA Interference Defective-1 (SID-1) gene, and methods of using the dsRNA to inhibit expression of SID-1.

### BACKGROUND OF THE INVENTION

RNA interference (RNAi) is a post-transcriptional silencing mechanism involving sequence-specific degradation of homologous mRNA mediated by double stranded RNA (dsRNA). Systemic RNA interference defective-1 (SID-1), a 776-amino acid transmembrane channel protein, originally identified in C. elegans, belongs to a novel, uncharacterized gene family that includes mammalian homologs (Feinberg E. H. et al. (2003) Science 301:1545-1547, Winston W. M. et al. (2002) Science 295:2456-2459). Systemic dsRNA uptake has been uniquely observed in C. elegans (see Svoboda P. (2004) Cytogenet Genome Res 105:422-434) due to expression of systemic RNA interference defective-1 (SID-1) (Feinberg E. H., et al. (2003) Science 301:1545-1547, Winston W. M., et al. (2002) Science 295:2456-2459). Ectopic expression of the C. elegans protein SID-1 in Drosophila cells enables passive cellular uptake of soaking dsRNA (Feinberg E. H., et al. (2003) Science 301:1545-1547). A recent report demonstrated that overexpression of a human SID-1 homolog, FLJ20174, in pancreatic ductal adenocarcinoma cells enhanced the passive uptake of siRNAs (Duxbury M. S. et al. (2005) Biochem Biophys Res Commun 331:459-463).

Despite significant advances in the field of RNAi and advances in the treatment of pathological processes, there remains a need for compositions and methods that can selectively and efficiently deliver agents to cells where silencing can then occur.

### SUMMARY OF THE INVENTION

The invention provides compositions containing double-stranded ribonucleic acid (dsRNA) and methods for inhibiting the expression of a SID-1 gene, such as in a cell or mammal. The invention also provides methods for identifying agents that target SID-1, e.g., oligonucleotides, such as dsRNAs, that target SID-1.

In one aspect, the invention features compositions containing an inhibitor of SID-1 expression where the inhibitor is a double stranded oligonucleotide, e.g., a double stranded RNA (dsRNA). In one embodiment, a dsRNA includes an RNA strand (the antisense strand) having a region which is less than 30 nucleotides in length, generally 18-30 nucleotides in length, and is substantially complementary to at least part of an mRNA transcript of the SID-1 target gene. In one embodiment, a dsRNA for inhibiting expression of the target gene includes at least two sequences that are complementary to each other. The dsRNA includes a sense strand having a first sequence and an antisense strand having a second sequence. The antisense strand includes a nucleotide sequence that is substantially complementary to at least part of an mRNA encoding a target gene, and the region of complementarity is less than 30 nucleotides in length, and at least 10 nucleotides in length. Generally, the dsRNA is 19 to 24, e.g., 19 to 21 nucleotides in length. In some embodiments the dsRNA is from about 10 to about 15 nucleotides in length, and in other embodiments the dsRNA is from about 25 to about 30 nucleotides in length. In one embodiment the strands are independently 18-30 nucleotides in length.

In another aspect, the invention features compositions containing an inhibitor of SID-1 expression where the inhibitor is a single-stranded oligonucleotide, e.g., a single-stranded DNA or RNA. In one embodiment, a single-stranded oligonucleotide includes a nucleotide sequence that is substantially complementary to at least part of an mRNA encoding a target gene, and the region of complementarity is less than 60 nucleotides in length, e.g., less than 30 nucleotides in length, and is at least 15 nucleotides in length. Generally, the single stranded oligonucleotides are 18 to 30, e.g., 19 to 21 nucleotides in length. In one embodiment the strand is 18-30 nucleotides in length. Single stranded oligonucleotides, e.g., single stranded DNA or RNA having less than 100% complementarity to the target mRNA are also embraced by the present invention.

The oligonucleotides featured herein, e.g., the single stranded and double stranded oligonucleotides featured herein, can include naturally occurring nucleotides or can include at least one modified nucleotide, such as a 2′-O-methyl modified nucleotide, a nucleotide having a non-phosphate backbone such as phosphorothioate and a terminal nucleotide linked to a cholesteryl derivative. Alternatively, the modified nucleotide may be chosen from the group of: a 2′-deoxy-2′-fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide, a phosphoramidate, and a non-natural base comprising nucleotide.

The oligonucleotides of the present invention may be preferably stabilized by one or more modifications to avoid degradation of the oligonucleotides. Possible modifications are phosphorothioate units, 2′-O-methyl RNA units, 2′-O-methoxy-ethyl RNA units, peptide nucleic acid units, N3′-P5′ phosphoroamidate DNA units, 2′ fluoro-ribo nucleic acid units, Locked nucleic acid units, morpholino phosphoroamidate nucleic acid units, cyclohexane nucleic acid units, tricyclonucleic acid units, 2′-O-alkylated nucleotide modifications, 2′-Deoxy-2′-fluoro modifications, 2,4-difluorotoluoyl modifications, 4′-thio ribose modifications, or boranophosphate modifications.

In one embodiment, the oligonucleotides featured herein include a first sequence that is selected from the group consisting of the sense sequences of Tables 3A, 3B, 4A and 4B, and a second sequence that is selected from the group consisting of the antisense sequences of Tables 3A, 3B, 4A and 4B. The dsRNA molecules featured herein can include naturally occurring nucleotides or can include at least one modified nucleotide, such as a 2′-O-methyl modified nucleotide, a nucleotide having a 5′-phosphorothioate group, and a terminal nucleotide linked to a cholesteryl derivative. Alternatively, the modified nucleotide may be chosen from the group of: a 2′-deoxy-2′-fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide, a phosphoramidate, and a non-natural base comprising nucleotide. Generally, such modified sequence will be based on a first sequence of said dsRNA selected from the group consisting of the sense sequences of Tables 3A, 3B, 4A and 4B, and a second sequence selected from the group consisting of the antisense sequences of Tables 3A, 3B, 4A and 4B.

In one aspect, the invention provides a method for inhibiting the expression of a SID-1 gene in a cell by performing the following steps:
(a) introducing into the cell a double-stranded ribonucleic acid (dsRNA), wherein the dsRNA includes at least two sequences that are complementary to each other. The dsRNA has a sense strand having a first sequence and an antisense strand having a second sequence; the antisense strand has a region of complementarity that is substantially complementary to at least a part of an mRNA encoding SID-1, and where the region of complementarity is less than 30 nucleotides in length, generally 19-24 nucleotides in length, and where the dsRNA, upon contact with a cell expressing SID-1, inhibits expression of a SID-1 gene by at least 40%;
and
(b) maintaining the cell produced in step (a) for a time sufficient to obtain degradation of the mRNA transcript of SID-1 gene, thereby inhibiting expression of a SID-1 gene in the cell.

In one embodiment, the method of reducing expression of the SID-1 gene in a cell is performed in vitro. In another embodiment, the method is performed in vivo.

In one embodiment, the SID-1 dsRNA featured in the invention reduces the amount of SID-1 mRNA present in a cultured cell, e.g., a primary cell or a transformed cell. In certain embodiments, the dsRNA reduces the amount of SID-1 mRNA in cultured human cells, such as human cells of epithelial origin, such as PC3 cells. In one embodiment, the dsRNA featured in the invention reduces the amount of SID-1 mRNA in cultured cells by at least 20%, 25%, 35%, 40%, 45%, 50%, 55%, 60%, 65%, 70%, 75%, or 80%.

In another aspect, the invention provides a cell containing at least one of the dsRNAs of the invention. The cell is generally a mammalian cell, such as a human or rodent cell. In one embodiment, the cell is a cultured human cell, such as a cultured epithelial cell, or embryonic kidney cell. In another embodiment, the cell is an isolated stem cell, e.g., a pluripotent or multipotent stem cell or a derivative thereof.

In yet another aspect, the invention features a method of identifying a dsRNA capable of inhibiting SID-1 expression in a cell. The method features, for example, contacting a candidate SID-1 dsRNA with a cell, such as a mammalian cell, for a time sufficient to inhibit SID-1 expression in the cell, and then assaying for SID-1 expression. In one embodiment, the candidate dsRNA is contacted with a human cell, such as a PC3 cell, for a time sufficient to inhibit SID-1 expression, and then SID-1 expression levels are assayed by reverse transcription coupled with polymerase chain reaction (RT-PCR).

In yet another aspect, the invention features a method for studying dsRNA uptake into a cell, e.g., a mammalian cell. In one embodiment, the effect of preincubation of the cell with a SID-1 dsRNA on the uptake of a second dsRNA is assayed, for example, by RT-PCR, branched DNA assay, Northern blot or Western blot analysis, or by a reporter gene system. For example, a cell is contacted with a SID-1 dsRNA for a period of time sufficient to cause a decrease in SID-1 expression levels, and then the cell is contacted with a second dsRNA, and then uptake of the second dsRNA is assayed by one of the above methods. In one embodiment, the second dsRNA targets a gene other than SID-1, such as an endogenous gene other than SID-1. In another embodiment, the second dsRNA targets an exogenously expressed gene, such as a gene other than SID-1. In one embodiment, the inhibition of SID-1 expression inhibits or decreases the amount of RNA interference (RNAi) that occurs in the cell.

In another aspect, the invention features a method of regulating dsRNA uptake into a cell, such as into a cell of a particular tissue. In one embodiment, a SID-1 dsRNA is targeted to a specific tissue, such as to the liver, and then a second dsRNA is administered, e.g., to a human or mouse. Accordingly, the second dsRNA will have a greater inhibitory effect on tissues other than the one targeted by the SID-1 dsRNA.

In another aspect, the SID-1 dsRNA is used as a tool to identify agents that enhance uptake of dsRNA. In one embodiment, SID-1 dsRNA is contacted with cells, such as cells in culture, to decrease dsRNA uptake efficiency. A library of agents (e.g., small molecule compounds, antibodies, aptamers, and the like) is screened to identify agents that enhance uptake of dsRNA, even in the presence of the SID-1 dsRNA.

In another aspect, the SID-1 dsRNA featured in the invention is used to identify genes involved in RNAi. In one embodiment, gene expression analysis (e.g., by microarrays or gene chips) is performed before and after contact of a cell with the SID-1 dsRNA.

In another aspect, the invention provides a cell containing a sequence capable of expressing a SID-1 dsRNA (e.g., a SID-1 dsRNA hair pin, or two separate antisense and sense strands of a dsRNA) in a cell, which inhibits expression of a SID-1 gene. In one embodiment, the dsRNA is expressed from a vector in the cell, and in another embodiment, the sequence expressing the dsRNA is stably integrated into the genome of the cell. In one embodiment, sequence includes a regulatory sequence operably linked to a nucleotide sequence that encodes at least one strand of one of the dsRNAs featured in the invention.

In another aspect, the invention features a non-human transgenic animal expressing a SID-1 dsRNA. In one embodiment the SID-1 dsRNA is expressed under a regulatable promoter, which can be activated in certain tissue-types. A second dsRNA administered to the animal will not inhibit gene expression, or will inhibit target gene expression to a lesser extent, in the tissues expressing the SID-1 dsRNA than in the tissues not expressing the SID-1 dsRNA. In some embodiment, the non-human animal is a rat, mouse, hamster, guinea pig, rabbit, dog, cat, goat, sheep, or pig.

In another aspect, the invention provides a double-stranded ribonucleic acid (dsRNA), wherein said dsRNA comprises a sense strand and an antisense strand, each less than 30 nucleotides in length, wherein said sense strand comprises at least 15 contiguous nucleotides from SEQ ID NO: 5 and wherein said antisense strand comprises at least 15 contiguous nucleotides from SEQ ID NO: 6, and wherein said antisense strand is complementary to at least 15 nucleotides of said sense strand and complementary to at least a part of a mRNA encoding Systemic RNA Interference Defective-1 (SID-1), and wherein said region of complementarity between said antisense strand and said mRNA is between 15 and 30 contiguous nucleotides in length. In certain embodiments, said part of a mRNA encoding SID-1 is the part starting at the nucleotide position corresponding to position 1200 in the human SID-1 transcript (NM_{—}017699.2) and ending at position 1229, or the part starting at position 1200 and ending at position 1218. In a related embodiment, the dsRNA comprises at least one modified nucleotide. In yet another related embodiment, the one or more modified nucleotides are chosen from the group consisting of: a 2′-O-methyl modified nucleotide, a nucleotide comprising a 5′-phosphorothioate group, and a terminal nucleotide linked to a cholesteryl derivative or dodecanoic acid bisdecylamide group. In other related embodiments, the modified nucleotide is chosen from the group consisting of: a 2′-deoxy-2′-fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide, a phosphoramidate, and a non-natural base comprising nucleotide. In other embodiments, the anti-SID-1 dsRNA comprises a phosphorothioate or a 2′-modified nucleotide.

In certain embodiments, the region of complementary between said antisense strand and said mRNA is at least 19 nucleotides in length, or between 19 and 21 nucleotides in length. In yet other embodiments, the dsRNA comprises a nucleotide overhang having 1, 2, 3 or 4 nucleotides. In certain related embodiments, the nucleotide overhang is at the 3′-end of the antisense strand of the dsRNA.

In one embodiment, the sense strand of said dsRNA consists of the sequence of SEQ ID NO: 435 and said antisense strand of said dsRNA consists of the sequence of SEQ ID NO: 436. In one embodiment, the dsRNA is agent AD-3947, consisting of oligonucleotides A-31031 and A-31032. In another embodiment, the dsRNA is agent AD-3946, agent AD-3962, or agent AD-3953.

In another aspect, the invention provides a double-stranded ribonucleic acid (dsRNA), wherein said dsRNA comprises a sense strand and an antisense strand, each less than 30 nucleotides in length, wherein said sense strand comprises at least 15 contiguous nucleotides from SEQ ID NO: 5 and wherein said antisense strand comprises at least 15 contiguous nucleotides from SEQ ID NO: 6, and wherein said antisense strand is complementary to at least 15 nucleotides of said sense strand and complementary to at least a part of a mRNA encoding Systemic RNA Interference Defective-1 (SID-1), and wherein said region of complementarity between said antisense strand and said mRNA is between 15 and 30 contiguous nucleotides in length, and wherein the dsRNA reduces the amount of SID-1 mRNA present in cultured human cells. In certain embodiments, the dsRNA reduces the amount of SID-1 mRNA in cultured PC3 cells after incubation with the dsRNA by more than 60% compared to cells that have not been incubated with the dsRNA.

The invention also provides a vector comprising a nucleotide sequence that encodes at least one strand of a dsRNA, wherein said dsRNA comprises a sense strand and an antisense strand, each less than 30 nucleotides in length, wherein said sense strand comprises at least 15 contiguous nucleotides from SEQ ID NO: 5 and wherein said antisense strand comprises at least 15 contiguous nucleotides from SEQ ID NO: 6, and wherein said antisense strand is complementary to at least 15 nucleotides of said sense strand and complementary to at least a part of a mRNA encoding Systemic RNA Interference Defective-1 (SID-1), and wherein said region of complementarity between said antisense strand and said mRNA is between 15 and 30 contiguous nucleotides in length. In certain embodiments, the vector is comprised by a cell.

The invention also provides a pharmaceutical composition for inhibiting the expression of SID-1, comprising one of the above-mentioned anti-SID-1 dsRNAs. In one embodiment, the sense strand of said dsRNA consists of the sequence of SEQ ID NO: 435 and said antisense strand of said dsRNA consists of the sequence of SEQ ID NO: 436.

In yet another embodiment, the invention provides a pharmaceutical composition for inhibiting SID-1 expression, comprising an anti-SID-1 dsRNA described above and a lipid formulation, e.g., a lipid formulation such as one of the formulations described herein.

In yet another embodiment, the invention provides a method of reducing the amount of SID-1 RNA in a cell comprising: (a) contacting the cell with an anti-SID-1 dsRNA described herein and (b) maintaining the contact established in step (a) for a time sufficient to mediate cleavage of SID-1 RNA in the cell, thereby reducing the amount of SID-1 RNA in the cell. In certain embodiments, the method is performed in vitro. In certain related embodiments, step (a) comprises introducing the dsRNA into the cell by lipofection. In certain related embodiments, TNF-α expression is not detectably increased following administration of said anti-SID-1 dsRNA. In yet other related embodiments, IFN-αexpression is increased less than 20% following administration of said anti-SID-1 dsRNA, as compared to a control dsRNA. In still other related embodiments, the IC50 of said anti-SID-1 dsRNA is less than 0.01 nM, less than 0.004 nM, or between 0.001 and 0.002 nM.

The invention also provides a method of inhibiting RNA interference (RNAi) in a cell comprising (a) contacting the cell with an anti-SID-1 dsRNA described herein, and (b) maintaining the contact established in step (a) for a time sufficient to inhibit uptake of a second dsRNA into the cell, thereby inhibiting RNAi in the cell. In certain related embodiments, TNF-α expression is not detectably increased following administration of said anti-SID-1 dsRNA. In yet other related embodiments, IFN-α expression is increased less than 20% following administration of said anti-SID-1 dsRNA, as compared to a control dsRNA. In still other related embodiments, the IC50 of said anti-SID-1 dsRNA is less than 0.01 nM, less than 0.004 nM, or between 0.001 and 0.002 nM.

The following detailed description discloses how to make and use the compositions containing dsRNAs to inhibit the expression of the SID-1 gene. The compositions featured in the invention include a dsRNA having an antisense strand comprising a region of complementarity which is less than 30 nucleotides in length, generally 19-24 nucleotides in length, and is substantially complementary to at least part of an RNA transcript of the SID-1 gene, together with an acceptable carrier. The compositions featured in the invention also include a dsRNA having an antisense strand having a region of complementarity which is less than 30 nucleotides in length, generally 19-24 nucleotides in length, and is substantially complementary to at least part of an RNA transcript of the SID-1 gene.

SID-2 is a transmembrane protein expressed in the intestine and localized to the apical (luminal) membrane. SID-2 is also involved in uptake of dsRNA from the environment. Thus, in one embodiment, the invention provides compositions and methods for targeting SID-2 and/or SID-1.

### BRIEF DESCRIPTION OF THE DRAWINGS

FIG. 1A depicts real-time RT-PCR results for single dose SID-1 knockdown using 5 nM or 25 nM duplexes in PC3 cells. FIG. 1B and FIG. 1C depict real-time RT-PCR results for single dose SID-1 knockdown using 5 nM (FIG. 1B) or 10 nM (FIG. 1C) duplex (PS).

FIG. 2 depicts SID-1 siRNA IFN-α/TNF-α cytokine stimulation results.

### DETAILED DESCRIPTION OF THE INVENTION

The invention provides dsRNAs and methods of using the dsRNAs for inhibiting the expression of a SID-1 gene in a cell or a mammal where the dsRNA targets a SID-1 gene.

The dsRNAs of the compositions featured herein include an RNA strand (the antisense strand) having a region which is less than 30 nucleotides in length, generally 19-24 nucleotides in length, and is substantially complementary to at least part of an mRNA transcript of a SID-1 gene. The use of these dsRNAs enables the targeted degradation of mRNAs of genes that are implicated in pathologies associated with SID-1 expression in mammals. Low dosages of SID-1 dsRNAs in particular can specifically and efficiently mediate RNAi, resulting in significant inhibition of expression of a SID-1 gene. Using cell-based assays, the present inventors have demonstrated that dsRNAs targeting SID-1 can specifically and efficiently mediate RNAi, resulting in significant inhibition of expression of a SID-1 gene.

### DEFINITIONS

For convenience, the meaning of certain terms and phrases used in the specification, examples, and appended claims, are provided below. If there is an apparent discrepancy between the usage of a term in other parts of this specification and its definition provided in this section, the definition in this section shall prevail.

“G,” “C,” “A” and “U” each generally stand for a nucleotide that contains guanine, cytosine, adenine, and uracil as a base, respectively. “T” and “dT” are used interchangeably herein and refer to a deoxyribonucleotide wherein the nucleobase is thymine, e.g., deoxyribothymine. However, it will be understood that the term “ribonucleotide” or “nucleotide” or “deoxyribonucleotide” can also refer to a modified nucleotide, as further detailed below, or a surrogate replacement moiety. The skilled person is well aware that guanine, cytosine, adenine, and uracil may be replaced by other moieties without substantially altering the base pairing properties of an oligonucleotide comprising a nucleotide bearing such replacement moiety. For example, without limitation, a nucleotide comprising inosine as its base may base pair with nucleotides containing adenine, cytosine, or uracil. Hence, nucleotides containing uracil, guanine, or adenine may be replaced in the nucleotide sequences of the invention by a nucleotide containing, for example, inosine. Sequences comprising such replacement moieties are embodiments of the invention.

As used herein, “SID-1” refers to Systemic RNA Interference Defective 1. SID-1 sequence can be found as RefSeq ID number: NM_{—}017699 (human), NM_{—}198034 (mouse), and NM_{—}221455 (rat). The human SID-1 gene is also known as F1120174, SIDT1 (SID1 transmembrane family member 1), Sidt1, and B830021E24Rik. A dsRNA featured in the invention can target a specific SID-1 isoform, e.g., a spliced or unspliced form, or a dsRNA of the invention can target multiple isoforms, e.g., by binding to a common region of the mRNA transcript. There are six known isoforms of human SID-1, and in some embodiments, a dsRNA featured in the invention will target one or two specific isoforms.

As used herein, a “SID-1 homolog” refers to a nucleic acid variant of SID-1, e.g., a variant of a SID-1 sequence described above. A member of the SID-1 family enhances uptake of dsRNA into cells. Calculations of “homology” between two sequences are performed as follows. The sequences are aligned for optimal comparison purposes (e.g., gaps can be introduced in one or both of a first and a second amino acid or nucleic acid sequence for optimal alignment, and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frameshift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences.

As used herein, “target sequence” refers to a contiguous portion of the nucleotide sequence of an mRNA molecule formed during the transcription of the SID-1 gene, including mRNA that is a product of RNA processing of a primary transcription product.

As used herein, the term “strand comprising a sequence” refers to an oligonucleotide comprising a chain of nucleotides that is described by the sequence referred to using the standard nucleotide nomenclature.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions may include: 400 mM NaCl, 40 mM PIPES pH 6.4, 1 mM EDTA, 50° C. or 70° C. for 12-16 hours followed by washing. Other conditions, such as physiologically relevant conditions as may be encountered inside an organism, can apply. The skilled person will be able to determine the set of conditions most appropriate for a test of complementarity of two sequences in accordance with the ultimate application of the hybridized nucleotides.

This includes base-pairing of the oligonucleotide or polynucleotide comprising the first nucleotide sequence to the oligonucleotide or polynucleotide comprising the second nucleotide sequence over the entire length of the first and second nucleotide sequence. Such sequences can be referred to as “fully complementary” with respect to each other herein. However, where a first sequence is referred to as “substantially complementary” with respect to a second sequence herein, the two sequences can be fully complementary, or they may form one or more, but generally not more than 4, 3 or 2 mismatched base pairs upon hybridization, while retaining the ability to hybridize under the conditions most relevant to their ultimate application. However, where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, may yet be referred to as “fully complementary” for the purposes of the invention.

“Complementary” sequences, as used herein, may also include, or be formed entirely from, non-Watson-Crick base pairs and/or base pairs formed from non-natural and modified nucleotides, in as far as the above requirements with respect to their ability to hybridize are fulfilled. Such non-Watson-Crick base pairs includes, but not limited to, G:U Wobble or Hoogstein base pairing.

The terms “complementary,” “fully complementary” and “substantially complementary” herein may be used with respect to the base matching between the sense strand and the antisense strand of a dsRNA, or between the antisense strand of a dsRNA and a target sequence, as will be understood from the context of their use.

As used herein, a polynucleotide which is “substantially complementary to at least part of” a messenger RNA (mRNA) refers to a polynucleotide which is substantially complementary to a contiguous portion of the mRNA of interest (e.g., encoding SID-1) including a 5′ UTR, an open reading frame, or a 3′ UTR. For example, a polynucleotide is complementary to at least a part of a SID-1 mRNA if the sequence is substantially complementary to a non-interrupted portion of an mRNA encoding SID-1.

The term “double-stranded RNA” or “dsRNA”, as used herein, refers to a complex of ribonucleic acid molecules, having a duplex structure comprising two anti-parallel and substantially complementary, as defined above, nucleic acid strands. In general, the majority of nucleotides of each strand are ribonucleotides, but as described in detail herein, each or both strands can also include at least one non-ribonucleotide, e.g., a deoxyribonucleotide and/or a modified nucleotide. In addition, as used in this specification, “dsRNA” may include chemical modifications to ribonucleotides, including substantial modifications at multiple nucleotides and including all types of modifications disclosed herein or known in the art. Any such modifications, as used in an siRNA type molecule, are encompassed by “dsRNA” for the purposes of this specification and the claims.

The two strands forming the duplex structure may be different portions of one larger RNA molecule, or they may be separate RNA molecules. Where the two strands are part of one larger molecule, and therefore are connected by an uninterrupted chain of nucleotides between the 3′-end of one strand and the 5′ end of the respective other strand forming the duplex structure, the connecting RNA chain is referred to as a “hairpin loop”. Where the two strands are connected covalently by means other than an uninterrupted chain of nucleotides between the 3′-end of one strand and the 5′ end of the respective other strand forming the duplex structure, the connecting structure is referred to as a “linker.” The RNA strands may have the same or a different number of nucleotides. The maximum number of base pairs is the number of nucleotides in the shortest strand of the dsRNA minus any overhangs that are present in the duplex. In addition to the duplex structure, a dsRNA may comprise one or more nucleotide overhangs.

As used herein, a “nucleotide overhang” refers to the unpaired nucleotide or nucleotides that protrude from the duplex structure of a dsRNA when a 3′-end of one strand of the dsRNA extends beyond the 5′-end of the other strand, or vice versa. “Blunt” or “blunt end” means that there are no unpaired nucleotides at that end of the dsRNA, i.e., no nucleotide overhang. A “blunt ended” dsRNA is a dsRNA that is double-stranded over its entire length, i.e., no nucleotide overhang at either end of the molecule.

The term “antisense strand” refers to the strand of a dsRNA which includes a region that is substantially complementary to a target sequence. As used herein, the term “region of complementarity” refers to the region on the antisense strand that is substantially complementary to a sequence, for example a target sequence, as defined herein. Where the region of complementarity is not fully complementary to the target sequence, the mismatches are most tolerated in the terminal regions and, if present, are generally in a terminal region or regions, e.g., within 6, 5, 4, 3, or 2 nucleotides of the 5′ and/or 3′ terminus.

The term “sense strand,” as used herein, refers to the strand of a dsRNA that includes a region that is substantially complementary to a region of the antisense strand.

“Introducing into a cell”, when referring to a dsRNA, means facilitating uptake or absorption into the cell, as is understood by those skilled in the art. Absorption or uptake of dsRNA can occur through unaided diffusive or active cellular processes, or by auxiliary agents or devices. The meaning of this term is not limited to cells in vitro; a dsRNA may also be “introduced into a cell,” wherein the cell is part of a living organism. In such instance, introduction into the cell will include the delivery to the organism. For example, for in vivo delivery, dsRNA can be injected into a tissue site or administered systemically. In vitro introduction into a cell includes methods known in the art such as electroporation and lipofection.

The terms “silence,” “inhibit the expression of,” “down-regulate the expression of,” “suppress the expression of,” and the like, in as far as they refer to the SID-1 gene, herein refer to the at least partial suppression of the expression of the SID-1 gene, as manifested by a reduction of the amount of SID-1 mRNA that may be isolated from or detected from a first cell or group of cells in which the SID-1 gene is transcribed and which has or have been treated such that the expression of the SID-1 gene is inhibited, as compared to a second cell or group of cells substantially identical to the first cell or group of cells but which has or have not been so treated (control cells). The degree of inhibition is usually expressed in terms of

$$\frac{( mRNA in control cells ) - ( mRNA in treated cells )}{( mRNA in control cells )} \cdot 100 \%$$

Alternatively, the degree of inhibition may be given in terms of a reduction of a parameter that is functionally linked to SID-1 gene expression, e.g., the amount of protein encoded by the SID-1 gene which is expressed by a cell, or the number of cells displaying a certain phenotype, e.g., apoptosis. In principle, SID-1 gene silencing may be determined in any cell expressing the target, either constitutively or by genomic engineering, and by any appropriate assay. For example, SID-1 silencing may be assayed in a mammalian cell, such as a human or mouse or rat cell. The cells can be in culture, and can be, for example, epithelial in origin. The cell may be pluripotent, such as a stem cell. SID-1 silencing can be measured by, for example, RT-PCR, branched DNA assay, or Northern or Western blot analysis.

For example, in certain instances, expression of the SID-1 gene is suppressed by at least about 5%, 10%, 15%, 20%, 25%, 30%, 35%, 40%, 45%, or 50% by administration of the double-stranded oligonucleotide of the invention. In some embodiments, the SID-1 gene is suppressed by at least about 60%, 70%, or 80% by administration of the double-stranded oligonucleotide of the invention. In some embodiments, the SID-1 gene is suppressed by at least about 85%, 90%, or 95% by administration of the double-stranded oligonucleotide of the invention. Tables 5A and 5B provide values for suppressing expression of SID-1 using various SID-1 dsRNA molecules at various concentrations.

As used herein in the context of SID-1 expression, the terms “treat,” “treatment,” and the like, refer to relief from or alleviation of pathological processes mediated by SID-1 expression. In the context of the present invention insofar as it relates to any of the other conditions recited herein below (other than pathological processes mediated by SID-1 expression), the terms “treat,” “treatment,” and the like mean to relieve or alleviate at least one symptom associated with such condition, or to slow or reverse the progression of such condition.

As used herein, the phrases “therapeutically effective amount” and “prophylactically effective amount” refer to an amount that provides a therapeutic benefit in the treatment, prevention, or management of pathological processes mediated by SID-1 expression or an overt symptom of pathological processes mediated by SID-1 expression. The specific amount that is therapeutically effective can be readily determined by an ordinary medical practitioner, and may vary depending on factors known in the art, such as, for example, the type of pathological processes mediated by SID-1 expression, the patient's history and age, the stage of pathological processes mediated by SID-1 expression, and the administration of other anti-pathological processes mediated by SID-1 expression agents.

As used herein, a “pharmaceutical composition” comprises a pharmacologically effective amount of a dsRNA and a pharmaceutically acceptable carrier. As used herein, “pharmacologically effective amount,” “therapeutically effective amount” or simply “effective amount” refers to that amount of an RNA effective to produce the intended pharmacological, therapeutic or preventive result. For example, if a given clinical treatment is considered effective when there is at least a 25% reduction in a measurable parameter associated with a disease or disorder, a therapeutically effective amount of a drug for the treatment of that disease or disorder is the amount necessary to effect at least a 25% reduction in that parameter.

The term “pharmaceutically acceptable carrier” refers to a carrier for administration of a therapeutic agent. Such carriers include, but are not limited to, saline, buffered saline, dextrose, water, glycerol, ethanol, and combinations thereof. The term specifically excludes cell culture medium. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

As used herein, a “transformed cell” is a cell into which a vector has been introduced from which a dsRNA molecule may be expressed.

Double-Stranded Ribonucleic Acid (dsRNA)

As described in more detail herein, the invention provides double-stranded ribonucleic acid (dsRNA) molecules for inhibiting the expression of a SID-1 gene in a cell or mammal, where the dsRNA includes an antisense strand having a region of complementarity which is complementary to at least a part of an mRNA formed in the expression of the SID-1 gene, and where the region of complementarity is less than 30 nucleotides in length, generally 19-24 nucleotides in length, and where said dsRNA, upon contact with a cell expressing said SID-1 gene, inhibits the expression of said SID-1 gene by at least 30% as assayed by, for example, a PCR or branched DNA (bDNA) based method.

The dsRNA includes two RNA strands that are sufficiently complementary to hybridize to form a duplex structure. One strand of the dsRNA (the antisense strand) includes a region of complementarity that is substantially complementary, and generally fully complementary, to a target sequence, derived from the sequence of an mRNA formed during the expression of the SID-1 gene, the other strand (the sense strand) includes a region that is complementary to the antisense strand, such that the two strands hybridize and form a duplex structure when combined under suitable conditions. Generally, the duplex structure is between 15 and 30, more generally between 18 and 25, yet more generally between 19 and 24, and most generally between 19 and 21 base pairs in length. Similarly, the region of complementarity to the target sequence is between 15 and 30, more generally between 18 and 25, yet more generally between 19 and 24, and most generally between 19, 20, or 21 nucleotides in length. In one embodiment the duplex is 19 base pairs in length. In another embodiment, the duplex is 21 base pairs in length. The dsRNA of the invention may further include one or more single-stranded nucleotide overhangs, which may additionally comprise one or more phosphorothioate linkages.

The dsRNA of the invention can include one or more single-stranded overhang(s) of one or more nucleotides. In one embodiment, at least one end of the dsRNA has a single-stranded nucleotide overhang of 1 to 4, generally 1 or 2 nucleotides. In another embodiment, the antisense strand of the dsRNA has 1-10 nucleotides overhangs each at the 3′ end and the 5′ end over the sense strand. In further embodiments, the sense strand of the dsRNA has 1-10 nucleotides overhangs each at the 3′ end and the 5′ end over the antisense strand.

A dsRNAs having at least one nucleotide overhang can have unexpectedly superior inhibitory properties than the blunt-ended counterpart. In some embodiments the presence of only one nucleotide overhang strengthens the interference activity of the dsRNA, without affecting its overall stability. A dsRNA having only one overhang has proven particularly stable and effective in vivo, as well as in a variety of cells, cell culture mediums, blood, and serum. Generally, the single-stranded overhang is located at the 3′-terminal end of the antisense strand or, alternatively, at the 3′-terminal end of the sense strand. The dsRNA can also have a blunt end, generally located at the 5′-end of the antisense strand. Such dsRNAs can have improved stability and inhibitory activity, thus allowing administration at low dosages, i.e., less than 5 mg/kg body weight of the recipient per day. Generally, the antisense strand of the dsRNA has a nucleotide overhang at the 3′-end, and the 5′-end is blunt. In another embodiment, one or more of the nucleotides in the overhang is replaced with a nucleoside thiophosphate.

The dsRNA can be synthesized by standard methods known in the art as further discussed below, e.g., by use of an automated DNA synthesizer, such as are commercially available from, for example, Biosearch, Applied Biosystems, Inc.

In a preferred embodiment, the SID-1 gene is the human SID-1 gene. In specific embodiments, the first sequence is a sense strand of the dsRNA that includes a sense sequence selected from those listed in Tables 3A, 3B, 4A and 4B, and the second sequence includes an antisense sequence selected from those listed in Tables 3A, 3B, 4A and 4B. Alternative antisense agents that target elsewhere in the target sequences of the antisense sequences provided in Tables 3A, 3B, 4A and 4B can be readily determined using the target sequence and the flanking SID-1 sequence.

The dsRNA will include at least two nucleotide sequences selected from the groups of sequences provided in Tables 3A, 3B, 4A and 4B. One of the two sequences is complementary to the other of the two sequences, with one of the sequences being substantially complementary to a sequence of an mRNA generated in the expression of the SID-1 gene. As such, the dsRNA will include two oligonucleotides, where one oligonucleotide is described as the sense strand in Tables 3A, 3B, 4A and 4B, and the second oligonucleotide is described as the antisense strand in Tables 3A, 3B, 4A and 4B.

The skilled person is well aware that dsRNAs comprising a duplex structure of between 20 and 23, but specifically 21, base pairs have been hailed as particularly effective in inducing RNA interference (Elbashir et al., EMBO 2001, 20:6877-6888). However, others have found that shorter or longer dsRNAs can be effective as well. In the embodiments described above, by virtue of the nature of the oligonucleotide sequences provided in Tables 3A, 3B, 4A and 4B, the dsRNAs of the invention can include at least one strand of a length of minimally 21 nt. It can be reasonably expected that shorter dsRNAs having one of the sequences of Tables 3A, 3B, 4A and 4B, minus only a few nucleotides on one or both ends, may be similarly effective as compared to the dsRNAs described above. Hence, dsRNAs having a partial sequence of at least 15, 16, 17, 18, 19, 20, or more contiguous nucleotides from one of the sequences of Tables 3A, 3B, 4A and 4B, and differing in their ability to inhibit the expression of the SID-1 gene in a cell-based assay as described herein below by not more than 5, 10, 15, 20, 25, or 30% inhibition from a dsRNA comprising the full sequence, are contemplated by the invention. Further, dsRNAs that cleave within the target sequence of an antisense sequence provided in Tables 3A, 3B, 4A and 4B can readily be made using the sequences provided.

In addition, the dsRNAs provided in Tables 3A, 3B, 4A and 4B identify a site in the SID-1 mRNA that is susceptible to RNAi based cleavage. As such the present invention further features dsRNAs that target within the sequence targeted by one of the agents of the present invention. As used herein a second dsRNA is said to target within the sequence of a first dsRNA if the second dsRNA cleaves the message anywhere within the mRNA that is complementary to the antisense strand of the first dsRNA. Such a second agent will generally consist of at least 15 contiguous nucleotides from one of the sequences provided in Tables 3A, 3B, 4A and 4B, coupled to additional nucleotide sequences taken from the region contiguous to the selected sequence in the SID-1 gene. For example, the last 15 nucleotides of a first sequence in Table 3A, 3B, 4A or 4B combined with the next 6 nucleotides from the target SID-1 gene produces a single strand agent of 21 nucleotides that is based on one of the sequences provided in Tables 3A, 3B, 4A and 4B.

The dsRNA of the invention can contain one or more mismatches to the target sequence. In a preferred embodiment, the dsRNA of the invention contains no more than 3 mismatches. If the antisense strand of the dsRNA contains mismatches to a target sequence, it is preferable that the area of mismatch not be located in the center of the region of complementarity. If the antisense strand of the dsRNA contains mismatches to the target sequence, it is preferable that the mismatch be restricted to 5 nucleotides from either end, for example 5, 4, 3, 2, or 1 nucleotide from either the 5′ or 3′ end of the region of complementarity. For example, for a 23 nucleotide dsRNA strand which is complementary to a region of the SID-1 gene, the dsRNA generally does not contain any mismatch within the central 13 nucleotides. The methods described within the invention can be used to determine whether a dsRNA containing a mismatch to a target sequence is effective in promoting the expression of the SID-1 gene. Consideration of the efficacy of dsRNAs with mismatches in promoting expression of the SID-1 gene is important, especially if the particular region of complementarity in the SID-1 gene is known to have polymorphic sequence variation within the population.

Modifications

In yet another embodiment, the dsRNA is chemically modified to enhance stability. The nucleic acids of the invention may be synthesized and/or modified by methods well established in the art, such as those described in “Current protocols in nucleic acid chemistry”, Beaucage, S. L. et al. (Edrs.), John Wiley & Sons, Inc., New York, N.Y., USA, which is hereby incorporated herein by reference. Specific examples of preferred dsRNA compounds useful in this invention include dsRNAs containing modified backbones or no natural internucleoside linkages. As defined in this specification, dsRNAs having modified backbones include those that retain a phosphorus atom in the backbone and those that do not have a phosphorus atom in the backbone. For the purposes of this specification, and as sometimes referenced in the art, modified dsRNAs that do not have a phosphorus atom in their internucleoside backbone can also be considered to be oligonucleosides.

Preferred modified dsRNA backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; and 5,625,050, each of which is herein incorporated by reference

Preferred modified dsRNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, each of which is herein incorporated by reference.

In other preferred dsRNA mimetics, both the sugar and the internucleoside linkage, i.e., the backbone, of the nucleotide units are replaced with novel groups. The base units are maintained for hybridization with an appropriate nucleic acid target compound. One such oligomeric compound, a dsRNA mimetic that has been shown to have excellent hybridization properties, is referred to as a peptide nucleic acid (PNA). In PNA compounds, the sugar backbone of an dsRNA is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

Other embodiments of the invention are dsRNAs with phosphorothioate backbones and oligonucleosides with heteroatom backbones, and in particular —CH₂—NH—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —N(CH₃)—CH₂—CH₂—[wherein the native phosphodiester backbone is represented as —O—P—O—CH₂—] of the above-referenced U.S. Pat. No. 5,489,677, and the amide backbones of the above-referenced U.S. Pat. No. 5,602,240. Also preferred are dsRNAs having morpholino backbone structures of the above-referenced U.S. Pat. No. 5,034,506.

Modified dsRNAs may also contain one or more substituted sugar moieties. Preferred dsRNAs comprise one of the following at the 2′ position: OH; F; O—-, S—, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred dsRNAs comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an dsRNA, or a group for improving the pharmacodynamic properties of an dsRNA, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxy-alkoxy group. A further preferred modification includes 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂, also described in examples herein below.

Other preferred modifications include 2′-methoxy (2′-OCH₃), 2′-aminopropoxy (2′-OCH₂CH₂CH₂NH₂) and 2′-fluoro (2′-F). Similar modifications may also be made at other positions on the dsRNA, particularly the 3′ position of the sugar on the 3′ terminal nucleotide or in 2′-5′ linked dsRNAs and the 5′ position of 5′ terminal nucleotide. DsRNAs may also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application, and each of which is herein incorporated by reference in its entirety.

DsRNAs may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further nucleobases include those disclosed in U.S. Pat. No. 3,687,808, those disclosed in The Concise Encyclopedia Of Polymer Science And Engineering, pages 858-859, Kroschwitz, J. L, ed. John Wiley & Sons, 1990, these disclosed by Englisch et al., Angewandte Chemie, International Edition, 1991, 30, 613, and those disclosed by Sanghvi, Y S., Chapter 15, DsRNA Research and Applications, pages 289-302, Crooke, S. T. and Lebleu, B., Ed., CRC Press, 1993. Certain of these nucleobases are particularly useful for increasing the binding affinity of the oligomeric compounds of the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., DsRNA Research and Applications, CRC Press, Boca Raton, 1993, pp. 276-278) and are presently preferred base substitutions, even more particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, each of which is herein incorporated by reference, and U.S. Pat. No. 5,750,692, also herein incorporated by reference.

Conjugates

Another modification of the dsRNAs of the invention involves chemically linking to the dsRNA one or more moieties or conjugates which enhance the activity, cellular distribution or cellular uptake of the dsRNA. Such moieties include but are not limited to lipid moieties such as a cholesterol moiety (Letsinger et al., Proc. Natl. Acid. Sci. USA, 199, 86, 6553-6556), cholic acid (Manoharan et al., Biorg. Med. Chem. Let., 1994 4 1053-1060), a thioether, e.g., beryl-5-tritylthiol (Manoharan et al., Ann. N.Y. Acad. Sci., 1992, 660, 306-309; Manoharan et al., Biorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-Hphosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyloxycholesterol moiety (Crooke et al., J. Pharmacol. Exp. Ther., 1996, 277, 923-937).

Representative U.S. patents that teach the preparation of such dsRNA conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

It is not necessary for all positions in a given compound to be uniformly modified, and in fact more than one of the aforementioned modifications may be incorporated in a single compound or even at a single nucleoside within an dsRNA. The present invention also includes dsRNA compounds which are chimeric compounds. “Chimeric” dsRNA compounds or “chimeras,” in the context of this invention, are dsRNA compounds, particularly dsRNAs, which contain two or more chemically distinct regions, each made up of at least one monomer unit, i.e., a nucleotide in the case of a dsRNA compound. These dsRNAs typically contain at least one region wherein the dsRNA is modified so as to confer upon the dsRNA increased resistance to nuclease degradation, increased cellular uptake, and/or increased binding affinity for the target nucleic acid. An additional region of the dsRNA may serve as a substrate for enzymes capable of cleaving RNA:DNA or RNA:RNA hybrids. By way of example, Rnase H is a cellular endonuclease which cleaves the RNA strand of an RNA:DNA duplex. Activation of Rnase H therefore results in cleavage of the RNA target, thereby greatly enhancing the efficiency of dsRNA inhibition of gene expression. Consequently, comparable results can often be obtained with shorter dsRNAs when chimeric dsRNAs are used, compared to phosphorothioate deoxy dsRNAs hybridizing to the same target region. Cleavage of the RNA target can be routinely detected by gel electrophoresis and, if necessary, associated nucleic acid hybridization techniques known in the art.

In certain instances, the dsRNA may be modified by a non-ligand group. A number of non-ligand molecules have been conjugated to dsRNAs in order to enhance the activity, cellular distribution or cellular uptake of the dsRNA, and procedures for performing such conjugations are available in the scientific literature. Such non-ligand moieties have included lipid moieties, such as cholesterol (Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-5-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923). Representative United States patents that teach the preparation of such dsRNA conjugates have been listed above. Typical conjugation protocols involve the synthesis of dsRNAs bearing an aminolinker at one or more positions of the sequence. The amino group is then reacted with the molecule being conjugated using appropriate coupling or activating reagents. The conjugation reaction may be performed either with the dsRNA still bound to the solid support or following cleavage of the dsRNA in solution phase. Purification of the dsRNA conjugate by HPLC typically affords the pure conjugate.

Vector Encoded dsRNAs

In another aspect of the invention, SID-1 specific dsRNA molecules that are expressed from transcription units inserted into DNA or RNA vectors (see, e.g., Couture, A, et al., TIG. (1996), 12:5-10; Skillern, A., et al., International PCT Publication No. WO 00/22113, Conrad, International PCT Publication No. WO 00/22114, and Conrad, U.S. Pat. No. 6,054,299). These transgenes can be introduced as a linear construct, a circular plasmid, or a viral vector, which can be incorporated and inherited as a transgene integrated into the host genome. The transgene can also be constructed to permit it to be inherited as an extrachromosomal plasmid (Gassmann, et al., Proc. Natl. Acad. Sci. USA (1995) 92:1292).

The individual strands of a dsRNA can be transcribed by promoters on two separate expression vectors and co-transfected into a target cell. Alternatively each individual strand of the dsRNA can be transcribed by promoters both of which are located on the same expression plasmid. In a preferred embodiment, a dsRNA is expressed as an inverted repeat joined by a linker polynucleotide sequence such that the dsRNA has a stem and loop structure.

The recombinant dsRNA expression vectors are generally DNA plasmids or viral vectors. dsRNA expressing viral vectors can be constructed based on, but not limited to, adeno-associated virus (for a review, see Muzyczka, et al., Curr. Topics Micro. Immunol. (1992) 158:97-129)); adenovirus (see, for example, Berkner, et al., BioTechniques (1998) 6:616), Rosenfeld et al. (1991, Science 252:431-434), and Rosenfeld et al. (1992), Cell 68:143-155)); or alphavirus as well as others known in the art. Retroviruses have been used to introduce a variety of genes into many different cell types, including epithelial cells, in vitro and/or in vivo (see, e.g., Eglitis, et al., Science (1985) 230:1395-1398; Danos and Mulligan, Proc. Natl. Acad. Sci. USA (1998) 85:6460-6464; Wilson et al., 1988, Proc. Natl. Acad. Sci. USA 85:3014-3018; Armentano et al., 1990, Proc. Natl. Acad. Sci. USA 87:61416145; Huber et al., 1991, Proc. Natl. Acad. Sci. USA 88:8039-8043; Ferry et al., 1991, Proc. Natl. Acad. Sci. USA 88:8377-8381; Chowdhury et al., 1991, Science 254:1802-1805; van Beusechem. Et al., 1992, Proc. Natl. Acad. Sci. USA 89:7640-19; Kay et al., 1992, Human Gene Therapy 3:641-647; Dai et al., 1992, Proc. Natl. Acad. Sci. USA 89:10892-10895; Hwu et al., 1993, J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573). Recombinant retroviral vectors capable of transducing and expressing genes inserted into the genome of a cell can be produced by transfecting the recombinant retroviral genome into suitable packaging cell lines such as PA317 and Psi-CRIP (Comette et al., 1991, Human Gene Therapy 2:5-10; Cone et al., 1984, Proc. Natl. Acad. Sci. USA 81:6349). Recombinant adenoviral vectors can be used to infect a wide variety of cells and tissues in susceptible hosts (e.g., rat, hamster, dog, and chimpanzee) (Hsu et al., 1992, J. Infectious Disease, 166:769), and also have the advantage of not requiring mitotically active cells for infection.

Any viral vector capable of accepting the coding sequences for the dsRNA molecule(s) to be expressed can be used, for example vectors derived from adenovirus (AV); adeno-associated virus (AAV); retroviruses (e.g, lentiviruses (LV), Rhabdoviruses, murine leukemia virus); herpes virus, and the like. The tropism of viral vectors can be modified by pseudotyping the vectors with envelope proteins or other surface antigens from other viruses, or by substituting different viral capsid proteins, as appropriate.

For example, lentiviral vectors of the invention can be pseudotyped with surface proteins from vesicular stomatitis virus (VSV), rabies, Ebola, Mokola, and the like. AAV vectors of the invention can be made to target different cells by engineering the vectors to express different capsid protein serotypes. For example, an AAV vector expressing a serotype 2 capsid on a serotype 2 genome is called AAV 2/2. This serotype 2 capsid gene in the AAV 2/2 vector can be replaced by a serotype 5 capsid gene to produce an AAV 2/5 vector. Techniques for constructing AAV vectors which express different capsid protein serotypes are within the skill in the art; see, e.g., Rabinowitz J E et al. (2002), J Virol 76:791-801, the entire disclosure of which is herein incorporated by reference.

Selection of recombinant viral vectors suitable for use in the invention, methods for inserting nucleic acid sequences for expressing the dsRNA into the vector, and methods of delivering the viral vector to the cells of interest are within the skill in the art. See, for example, Dornburg R (1995), Gene Therap. 2: 301-310; Eglitis M A (1988), Biotechniques 6: 608-614; Miller A D (1990), Hum Gene Therap. 1: 5-14; Anderson W F (1998), Nature 392: 25-30; and Rubinson D A et al., Nat. Genet. 33: 401-406, the entire disclosures of which are herein incorporated by reference.

Preferred viral vectors are those derived from AV and AAV. In a particularly preferred embodiment, the dsRNA of the invention is expressed as two separate, complementary single-stranded RNA molecules from a recombinant AAV vector having, for example, either the U6 or H1 RNA promoters, or the cytomegalovirus (CMV) promoter.

A suitable AV vector for expressing the dsRNA of the invention, a method for constructing the recombinant AV vector, and a method for delivering the vector into target cells, are described in Xia H et al. (2002), Nat. Biotech. 20: 1006-1010.

Suitable AAV vectors for expressing the dsRNA of the invention, methods for constructing the recombinant AV vector, and methods for delivering the vectors into target cells are described in Samulski R et al. (1987), J. Virol. 61: 3096-3101; Fisher K J et al. (1996), J. Virol, 70: 520-532; Samulski R et al. (1989), J. Virol. 63: 3822-3826; U.S. Pat. No. 5,252,479; U.S. Pat. No. 5,139,941; International Patent Application No. WO 94/13788; and International Patent Application No. WO 93/24641, the entire disclosures of which are herein incorporated by reference.

The promoter driving dsRNA expression in either a DNA plasmid or viral vector of the invention may be a eukaryotic RNA polymerase I (e.g. ribosomal RNA promoter), RNA polymerase II (e.g. CMV early promoter or actin promoter or U1 snRNA promoter) or generally RNA polymerase III promoter (e.g. U6 snRNA or 7SK RNA promoter) or a prokaryotic promoter, for example the T7 promoter, provided the expression plasmid also encodes T7 RNA polymerase required for transcription from a T7 promoter. The promoter can also direct transgene expression to the pancreas (see, e.g., the insulin regulatory sequence for pancreas (Bucchini et al., 1986, Proc. Natl. Acad. Sci. USA 83:2511-2515)).

In addition, expression of the transgene can be precisely regulated, for example, by using an inducible regulatory sequence and expression systems such as a regulatory sequence that is sensitive to certain physiological regulators, e.g., circulating glucose levels, or hormones (Docherty et al., 1994, FASEB J. 8:20-24). Such inducible expression systems, suitable for the control of transgene expression in cells or in mammals include regulation by ecdysone, by estrogen, progesterone, tetracycline, chemical inducers of dimerization, and isopropyl-beta-D1-thiogalactopyranoside (EPTG). A person skilled in the art would be able to choose the appropriate regulatory/promoter sequence based on the intended use of the dsRNA transgene.

Generally, recombinant vectors capable of expressing dsRNA molecules are delivered as described below, and persist in target cells. Alternatively, viral vectors can be used that provide for transient expression of dsRNA molecules. Such vectors can be repeatedly administered as necessary. Once expressed, the dsRNAs bind to target RNA and modulate its function or expression. Delivery of dsRNA expressing vectors can be systemic, such as by intravenous or intramuscular administration, by administration to target cells ex-planted from the patient followed by reintroduction into the patient, or by any other means that allows for introduction into a desired target cell.

dsRNA expression DNA plasmids are typically transfected into target cells as a complex with cationic lipid carriers (e.g. Oligofectamine) or non-cationic lipid-based carriers (e.g. Transit-TKO™). Multiple lipid transfections for dsRNA-mediated knockdowns targeting different regions of a single SID-1 gene or multiple SID-1 genes over a period of a week or more are also contemplated by the invention. Successful introduction of the vectors of the invention into host cells can be monitored using various known methods. For example, transient transfection. Can be signaled with a reporter, such as a fluorescent marker, such as Green Fluorescent Protein (GFP). Stable transfection of ex vivo cells can be ensured using markers that provide the transfected cell with resistance to specific environmental factors (e.g., antibiotics and drugs), such as hygromycin B resistance.

The SID-1 specific dsRNA molecules can also be inserted into vectors and used as gene therapy vectors for human patients. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can include a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

Pharmaceutical Compositions Comprising dsRNA

In one embodiment, the invention provides pharmaceutical compositions comprising a dsRNA, as described herein, and a pharmaceutically acceptable carrier. The pharmaceutical composition comprising the dsRNA is useful for treating a disease or disorder associated with the expression or activity of the SID-1 gene, such as pathological processes mediated by SID-1 expression. Such pharmaceutical compositions are formulated based on the mode of delivery. One example is compositions that are formulated for systemic administration via parenteral delivery.

The pharmaceutical compositions featured herein are administered in dosages sufficient to inhibit expression of the SID-1 genes. In general, a suitable dose of dsRNA will be in the range of 0.01 to 200.0 milligrams per kilogram body weight of the recipient per day, generally in the range of 1 to 50 mg per kilogram body weight per day. For example, the dsRNA can be administered at 0.01 mg/kg, 0.05 mg/kg, 0.5 mg/kg, 1 mg/kg, 1.5 mg/kg, 2 mg/kg, 3 mg/kg, 5.0 mg/kg, 10 mg/kg, 20 mg/kg, 30 mg/kg, 40 mg/kg, or 50 mg/kg per single dose. The pharmaceutical composition may be administered once daily or the dsRNA may be administered as two, three, or more sub-doses at appropriate intervals throughout the day or even using continuous infusion or delivery through a controlled release formulation. In that case, the dsRNA contained in each sub-dose must be correspondingly smaller in order to achieve the total daily dosage. The dosage unit can also be compounded for delivery over several days, e.g., using a conventional sustained release formulation which provides sustained release of the dsRNA over a several day period. Sustained release formulations are well known in the art and are particularly useful for delivery of agents at a particular site, such as could be used with the agents of the present invention. In this embodiment, the dosage unit contains a corresponding multiple of the daily dose.

The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition can include a single treatment or a series of treatments. Estimates of effective dosages and in vivo half-lives for the individual dsRNAs encompassed by the invention can be made using conventional methodologies or on the basis of in vivo testing using an appropriate animal model, as described elsewhere herein.

Advances in mouse genetics have generated a number of mouse models for the study of various human diseases, such as pathological processes mediated by SID-1 expression. Such models are used for in vivo testing of dsRNA, as well as for determining a therapeutically effective dose.

The data obtained from cell culture assays and animal studies can be used in formulating a range of dosage for use in humans. The dosage of compositions featured in the invention lies generally within a range of circulating concentrations that include the ED50 with little or no toxicity. The dosage may vary within this range depending upon the dosage form employed and the route of administration utilized. For any compound used in the methods featured in the invention, the therapeutically effective dose can be estimated initially from cell culture assays. A dose may be formulated in animal models to achieve a circulating plasma concentration range of the compound or, when appropriate, of the polypeptide product of a target sequence (e.g., achieving a decreased concentration of the polypeptide) that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The dsRNAs featured in the invention can be administered in combination with other known agents effective in treatment of pathological processes mediated by target gene expression. In any event, the administering physician can adjust the amount and timing of dsRNA administration on the basis of results observed using standard measures of efficacy known in the art or described herein.

Administration

The present invention also includes pharmaceutical compositions and formulations which include the dsRNA compounds of the invention. The pharmaceutical compositions of the present invention may be administered in a number of ways depending upon whether local or systemic treatment is desired and upon the area to be treated. Administration may be topical, pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. Preferred topical formulations include those in which the dsRNAs of the invention are in admixture with a topical delivery agent such as lipids, liposomes, fatty acids, fatty acid esters, steroids, chelating agents and surfactants. Preferred lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA). DsRNAs of the invention may be encapsulated within liposomes or may form complexes thereto, in particular to cationic liposomes. Alternatively, dsRNAs may be complexed to lipids, in particular to cationic lipids. Preferred fatty acids and esters include but are not limited arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C₁₋₁₀ alkyl ester (e.g. isopropylmyristate IPM), monoglyceride, diglyceride or pharmaceutically acceptable salt thereof. Topical formulations are described in detail in U.S. patent application Ser. No. 09/315,298 filed on May 20, 1999 which is incorporated herein by reference in its entirety.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Preferred oral formulations are those in which dsRNAs of the invention are administered in conjunction with one or more penetration enhancers surfactants and chelators. Preferred surfactants include fatty acids and/or esters or salts thereof, bile acids and/or salts thereof. Preferred bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate and sodium glycodihydrofusidate. Preferred fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g. sodium). Also preferred are combinations of penetration enhancers, for example, fatty acids/salts in combination with bile acids/salts. A particularly preferred combination is the sodium salt of lauric acid, capric acid and UDCA. Further penetration enhancers include polyoxyethylene-9-lauryl ether, polyoxyethylene-20-cetyl ether. DsRNAs of the invention may be delivered orally, in granular form including sprayed dried particles, or complexed to form micro or nanoparticles. DsRNA complexing agents include poly-amino acids; polyimines; polyacrylates; polyalkylacrylates, polyoxethanes, polyalkylcyanoacrylates; cationized gelatins, albumins, starches, acrylates, polyethyleneglycols (PEG) and starches; polyalkylcyanoacrylates; DEAE-derivatized polyimines, pollulans, celluloses and starches. Particularly preferred complexing agents include chitosan, N-trimethylchitosan, poly-L-lysine, polyhistidine, polyornithine, polyspermines, protamine, polyvinylpyridine, polythiodiethylaminomethylethylene P(TDAE), polyaminostyrene (e.g. p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG). Oral formulations for dsRNAs and their preparation are described in detail in U.S. application. Ser. No. 08/886,829 (filed Jul. 1, 1997), Ser. No. 09/108,673 (filed Jul. 1, 1998), Ser. No. 09/256,515 (filed Feb. 23, 1999), Ser. No. 09/082,624 (filed May 21, 1998) and Ser. No. 09/315,298 (filed May 20, 1999), each of which is incorporated herein by reference in their entirety.

Compositions and formulations for parenteral, intrathecal, intraventricular or intrahepatic administration may include sterile aqueous solutions which may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids. Particularly preferred are formulations that target the liver when treating hepatic disorders such as hepatic carcinoma.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product. The compositions of the present invention may be formulated into any of many possible dosage forms such as, but not limited to, tablets, capsules, gel capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

Emulsions

The compositions of the present invention may be prepared and formulated as emulsions. Emulsions are typically heterogenous systems of one liquid dispersed in another in the form of droplets usually exceeding 0.1 .mu.m in diameter (Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems comprising two immiscible liquid phases intimately mixed and dispersed with each other. In general, emulsions may be of either the water-in-oil (w/o) or the oil-in-water (o/w) variety. When an aqueous phase is finely divided into and dispersed as minute droplets into a bulk oily phase, the resulting composition is called a water-in-oil (w/o) emulsion. Alternatively, when an oily phase is finely divided into and dispersed as minute droplets into a bulk aqueous phase, the resulting composition is called an oil-in-water (o/w) emulsion. Emulsions may contain additional components in addition to the dispersed phases, and the active drug which may be present as a solution in either the aqueous phase, oily phase or itself as a separate phase. Pharmaceutical excipients such as emulsifiers, stabilizers, dyes, and anti-oxidants may also be present in emulsions as needed. Pharmaceutical emulsions may also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous phase provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion may be a semisolid or a solid, as is the case of emulsion-style ointment bases and creams. Other means of stabilizing emulsions entail the use of emulsifiers that may be incorporated into either phase of the emulsion. Emulsifiers may broadly be classified into four categories: synthetic surfactants, naturally occurring emulsifiers, absorption bases, and finely dispersed solids (Idson, in Pharmaceutical Dosage Forms, Lieberman, Rieger and Banker (Eds.), 1988, Marcel Dekker, Inc., New York, N.Y., volume 1, p. 199).

Synthetic surfactants, also known as surface active agents, have found wide applicability in the formulation of emulsions and have been reviewed in the literature (Rieger, in Pharmaceutical Dosage Forms, Lieberman, Rieger and Banker (Eds.), 1988, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants may be classified into different classes based on the nature of the hydrophilic group: nonionic, anionic, cationic and amphoteric (Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that may readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used may be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

The application of emulsion formulations via dermatological, oral and parenteral routes and methods for their manufacture have been reviewed in the literature (Idson, in Pharmaceutical Dosage Forms, Lieberman, Rieger and Banker (Eds.), 1988, Marcel Dekker, Inc., New York, N.Y., volume 1, p. 199). Emulsion formulations for oral delivery have been very widely used because of ease of formulation, as well as efficacy from an absorption and bioavailability standpoint (Rosoff, in Pharmaceutical Dosage Forms, Lieberman, Rieger and Banker (Eds.), 1988, Marcel Dekker, Inc., New York, N.Y., volume 1, p. 245; Idson, in Pharmaceutical Dosage Forms, Lieberman, Rieger and Banker (Eds.), 1988, Marcel Dekker, Inc., New York, N.Y., volume 1, p. 199). Mineral-oil base laxatives, oil-soluble vitamins and high fat nutritive preparations are among the materials that have commonly been administered orally as o/w emulsions.

In one embodiment of the present invention, the compositions of dsRNAs and nucleic acids are formulated as microemulsions. A microemulsion may be defined as a system of water, oil and amphiphile which is a single optically isotropic and thermodynamically stable liquid solution (Rosoff, in Pharmaceutical Dosage Forms, Lieberman, Rieger and Banker (Eds.), 1988, Marcel Dekker, Inc., New York, N.Y., volume 1, p. 245). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 185-215). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Schott, in Remington's Pharmaceutical Sciences, Mack Publishing Co., Easton, Pa., 1985, p. 271).

The phenomenological approach utilizing phase diagrams has been extensively studied and has yielded a comprehensive knowledge, to one skilled in the art, of how to formulate microemulsions (Rosoff, in Pharmaceutical Dosage Forms, Lieberman, Rieger and Banker (Eds.), 1988, Marcel Dekker, Inc., New York, N.Y., volume 1, p. 245; Block, in Pharmaceutical Dosage Forms, Lieberman, Rieger and Banker (Eds.), 1988, Marcel Dekker, Inc., New York, N.Y., volume 1, p. 335). Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monoleate (PO310), hexaglycerol pentaoleate (PO500), decaglycerol monocaprate (MCA750), decaglycerol monooleate (MO750), decaglycerol sequioleate (SO750), decaglycerol decaoleate (DAO750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions may, however, be prepared without the use of cosurfactants and alcohol-free self-emulsifying microemulsion systems are known in the art. The aqueous phase may typically be, but is not limited to, water, an aqueous solution of the drug, glycerol, PEG300, PEG400, polyglycerols, propylene glycols, and derivatives of ethylene glycol. The oil phase may include, but is not limited to, materials such as Captex 300, Captex 355, Capmul MCM, fatty acid esters, medium chain (C₈-C₁₂) mono, di, and tri-glycerides, polyoxyethylated glyceryl fatty acid esters, fatty alcohols, polyglycolized glycerides, saturated polyglycolized C8-C10 glycerides, vegetable oils and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (Constantinides et al., Pharmaceutical Research, 1994, 11, 1385-1390; Ritschel, Meth. Find. Exp. Clin. Pharmacol., 1993, 13, 205). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (Constantinides et al., Pharmaceutical Research, 1994, 11, 1385; Ho et al., J. Pharm. Sci., 1996, 85, 138-143). Often microemulsions may form spontaneously when their components are brought together at ambient temperature. This may be particularly advantageous when formulating thermolabile drugs, peptides or dsRNAs. Microemulsions have also been effective in the transdermal delivery of active components in both cosmetic and pharmaceutical applications. It is expected that the microemulsion compositions and formulations of the present invention will facilitate the increased systemic absorption of dsRNAs and nucleic acids from the gastrointestinal tract, as well as improve the local cellular uptake of dsRNAs and nucleic acids.

Microemulsions of the present invention may also contain additional components and additives such as sorbitan monostearate (Grill 3), Labrasol, and penetration enhancers to improve the properties of the formulation and to enhance the absorption of the dsRNAs and nucleic acids of the present invention. Penetration enhancers used in the microemulsions of the present invention may be classified as belonging to one of five broad categories—surfactants, fatty acids, bile salts, chelating agents, and non-chelating non-surfactants (Lee et al., Critical Reviews in Therapeutic Drug Carrier Systems, 1991, p. 92). Each of these classes has been discussed above.

Liposomal Formulations

There are many organized surfactant structures besides microemulsions that have been studied and used for the formulation of drugs. These include monolayers, micelles, bilayers and vesicles. Vesicles, such as liposomes, have attracted great interest because of their specificity and the duration of action they offer from the standpoint of drug delivery. As used in the present invention, the term “liposome” means a vesicle composed of amphiphilic lipids arranged in a spherical bilayer or bilayers.

Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are taken up by macrophages in vivo.

In order to cross intact mammalian skin, lipid vesicles must pass through a series of fine pores, each with a diameter less than 50 nm, under the influence of a suitable transdermal gradient. Therefore, it is desirable to use a liposome which is highly deformable and able to pass through such fine pores.

Further advantages of liposomes include; liposomes obtained from natural phospholipids are biocompatible and biodegradable; liposomes can incorporate a wide range of water and lipid soluble drugs; liposomes can protect encapsulated drugs in their internal compartments from metabolism and degradation (Rosoff, in Pharmaceutical Dosage Forms, Lieberman, Rieger and Banker (Eds.), 1988, Marcel Dekker, Inc., New York, N.Y., volume 1, p. 245). Important considerations in the preparation of liposome formulations are the lipid surface charge, vesicle size and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes and as the merging of the liposome and cell progresses, the liposomal contents are emptied into the cell where the active agent may act.

Liposomal formulations have been the focus of extensive investigation as the mode of delivery for many drugs. There is growing evidence that for topical administration, liposomes present several advantages over other formulations. Such advantages include reduced side-effects related to high systemic absorption of the administered drug, increased accumulation of the administered drug at the desired target, and the ability to administer a wide variety of drugs, both hydrophilic and hydrophobic, into the skin.

Several reports have detailed the ability of liposomes to deliver agents including high-molecular weight DNA into the skin. Compounds including analgesics, antibodies, hormones and high-molecular weight DNAs have been administered to the skin. The majority of applications resulted in the targeting of the upper epidermis

Liposomes fall into two broad classes. Cationic liposomes are positively charged liposomes which interact with the negatively charged DNA molecules to form a stable complex. The positively charged DNA/liposome complex binds to the negatively charged cell surface and is internalized in an endosome. Due to the acidic pH within the endosome, the liposomes are ruptured, releasing their contents into the cell cytoplasm (Wang et al., Biochem. Biophys. Res. Commun., 1987, 147, 980-985).

Liposomes which are pH-sensitive or negatively-charged, entrap DNA rather than complex with it. Since both the DNA and the lipid are similarly charged, repulsion rather than complex formation occurs. Nevertheless, some DNA is entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 1992, 19, 269-274).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Several studies have assessed the topical delivery of liposomal drug formulations to the skin. Application of liposomes containing interferon to guinea pig skin resulted in a reduction of skin herpes sores while delivery of interferon via other means (e.g., as a solution or as an emulsion) were ineffective (Weiner et al., Journal of Drug Targeting, 1992, 2, 405-410). Further, an additional study tested the efficacy of interferon administered as part of a liposomal formulation to the administration of interferon using an aqueous system, and concluded that the liposomal formulation was superior to aqueous administration (du Plessis et al., Antiviral Research, 1992, 18, 259-265).

Non-ionic liposomal systems have also been examined to determine their utility in the delivery of drugs to the skin, in particular systems comprising non-ionic surfactant and cholesterol. Non-ionic liposomal formulations comprising Novasome™ I (glyceryl dilaurate/cholesterol/po-lyoxyethylene-10-stearyl ether) and Novasome™ II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporin-A into different layers of the skin (Hu et al. S.T.P. Pharma. Sci., 1994, 4, 6, 466).

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside G_{M1}, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., FEBS Letters, 1987, 223, 42; Wu et al., Cancer Research, 1993, 53, 3765).

Various liposomes comprising one or more glycolipids are known in the art. Papahadjopoulos et al. (Ann. N.Y. Acad. Sci., 1987, 507, 64) reported the ability of monosialoganglioside G_{M1}, galactocerebroside sulfate and phosphatidylinositol to improve blood half-lives of liposomes. These findings were expounded upon by Gabizon et al. (Proc. Natl. Acad. Sci. U.S.A., 1988, 85, 6949). U.S. Pat. No. 4,837,028 and WO 88/04924, both to Allen et al., disclose liposomes comprising (1) sphingomyelin and (2) the ganglioside G_{M1} or a galactocerebroside sulfate ester. U.S. Pat. No. 5,543,152 (Webb et al.) discloses liposomes comprising sphingomyelin. Liposomes comprising 1,2-sn-dimyristoylphosphat-idylcholine are disclosed in WO 97/13499 (Lim et al).

Many liposomes comprising lipids derivatized with one or more hydrophilic polymers, and methods of preparation thereof, are known in the art. Sunamoto et al. (Bull. Chem. Soc. Jpn., 1980, 53, 2778) described liposomes comprising a nonionic detergent, 2C_{1215G}, that contains a PEG moiety. Illum et al. (FEBS Lett., 1984, 167, 79) noted that hydrophilic coating of polystyrene particles with polymeric glycols results in significantly enhanced blood half-lives. Synthetic phospholipids modified by the attachment of carboxylic groups of polyalkylene glycols (e.g., PEG) are described by Sears (U.S. Pat. Nos. 4,426,330 and 4,534,899). Klibanov et al. (FEBS Lett., 1990, 268, 235) described experiments demonstrating that liposomes comprising phosphatidylethanolamine (PE) derivatized with PEG or PEG stearate have significant increases in blood circulation half-lives. Blume et al. (Biochimica et Biophysica Acta, 1990, 1029, 91) extended such observations to other PEG-derivatized phospholipids, e.g., DSPE-PEG, formed from the combination of distearoylphosphatidylethanolamine (DSPE) and PEG. Liposomes having covalently bound PEG moieties on their external surface are described in European Patent No. EP 0 445 131 B1 and WO 90/04384 to Fisher. Liposome compositions containing 1-20 mole percent of PE derivatized with PEG, and methods of use thereof, are described by Woodle et al. (U.S. Pat. Nos. 5,013,556 and 5,356,633) and Martin et al. (U.S. Pat. No. 5,213,804 and European Patent No. EP 0 496 813 B1). Liposomes comprising a number of other lipid-polymer conjugates are disclosed in WO 91/05545 and U.S. Pat. No. 5,225,212 (both to Martin et al.) and in WO 94/20073 (Zalipsky et al.) Liposomes comprising PEG-modified ceramide lipids are described in WO 96/10391 (Choi et al). U.S. Pat. No. 5,540,935 (Miyazaki et al.) and U.S. Pat. No. 5,556,948 (Tagawa et al.) describe PEG-containing liposomes that can be further derivatized with functional moieties on their surfaces.

A number of liposomes comprising nucleic acids are known in the art. WO 96/40062 to Thierry et al. discloses methods for encapsulating high molecular weight nucleic acids in liposomes. U.S. Pat. No. 5,264,221 to Tagawa et al. discloses protein-bonded liposomes and asserts that the contents of such liposomes may include a dsRNA. U.S. Pat. No. 5,665,710 to Rahman et al. describes certain methods of encapsulating oligodeoxynucleotides in liposomes. WO 97/04787 to Love et al. discloses liposomes comprising dsRNAs targeted to the raf gene.

Transfersomes are yet another type of liposomes, and are highly deformable lipid aggregates which are attractive candidates for drug delivery vehicles. Transfersomes may be described as lipid droplets which are so highly deformable that they are easily able to penetrate through pores which are smaller than the droplet. Transfersomes are adaptable to the environment in which they are used, e.g., they are self-optimizing (adaptive to the shape of pores in the skin), self-repairing, frequently reach their targets without fragmenting, and often self-loading. To make transfersomes it is possible to add surface edge-activators, usually surfactants, to a standard liposomal composition. Transfersomes have been used to deliver serum albumin to the skin. The transfersome-mediated delivery of serum albumin has been shown to be as effective as subcutaneous injection of a solution containing serum albumin.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (Rieger, in Pharmaceutical Dosage Forms, Marcel Dekker, Inc., New York, N.Y., 1988, p. 285).

Nucleic Acid Lipid Particles

In one embodiment, a dsRNA featured in the invention is fully encapsulated in the lipid formulation to form a SPLP, pSPLP, SNALP, or other nucleic acid-lipid particle. As used herein, the term “SNALP” refers to a stable nucleic acid-lipid particle, including SPLP. As used herein, the term “SPLP” refers to a nucleic acid-lipid particle comprising plasmid DNA encapsulated within a lipid vesicle. SNALPs and SPLPs typically contain a cationic lipid, a non-cationic lipid, and a lipid that prevents aggregation of the particle (e.g., a PEG-lipid conjugate). SNALPs and SPLPs are extremely useful for systemic applications, as they exhibit extended circulation lifetimes following intravenous (i.v.) injection and accumulate at distal sites (e.g., sites physically separated from the administration site). SPLPs include “pSPLP,” which include an encapsulated condensing agent-nucleic acid complex as set forth in PCT Publication No. WO 00/03683. The particles of the present invention typically have a mean diameter of about 50 nm to about 150 nm, more typically about 60 nm to about 130 nm, more typically about 70 nm to about 110 nm, most typically about 70 to about 90 nm, and are substantially nontoxic. In addition, the nucleic acids when present in the nucleic acid-lipid particles of the present invention are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; and PCT Publication No. WO 96/40964.

In one embodiment, the lipid to drug ratio (mass/mass ratio) (e.g., lipid to dsRNA ratio) will be in the range of from about 1:1 to about 50:1, from about 1:1 to about 25:1, from about 3:1 to about 15:1, from about 4:1 to about 10:1, from about 5:1 to about 9:1, or about 6:1 to about 9:1.

The cationic lipid may be, for example, N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 2,2-Dilinoleyl-4-dimethylaminomethyl-[1,3]-dioxolane (DLin-K-DMA) or analogs thereof, or a mixture thereof. The cationic lipid may comprise from about 20 mol % to about 50 mol % or about 40 mol % of the total lipid present in the particle.

In another embodiment, the compound 2,2-Dilinoleyl-4-dimethylaminoethyl-[1,3]-dioxolane can be used to prepare lipid-siRNA nanoparticles. Synthesis of 2,2-Dilinoleyl-4-dimethylaminoethyl-[1,3]-dioxolane is described in U.S. provisional patent application No. 61/107,998 filed on Oct. 23, 2008, which is herein incorporated by reference.

In one embodiment, the lipid-siRNA particle includes 40% 2,2-Dilinoleyl-4-dimethylaminoethyl-[1,3]-dioxolane: 10% DSPC: 40% Cholesterol: 10% PEG-C-DOMG (mole percent) with a particle size of 63.0±20 nm and a 0.027 siRNA/Lipid Ratio.

The non-cationic lipid may be an anionic lipid or a neutral lipid including, but not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. The non-cationic lipid may be from about 5 mol % to about 90 mol %, about 10 mol %, or about 58 mol % if cholesterol is included, of the total lipid present in the particle.

The conjugated lipid that inhibits aggregation of particles may be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate may be, for example, a PEG-dilauryloxypropyl (Ci₂), a PEG-dimyristyloxypropyl (Ci₄), a PEG-dipalmityloxypropyl (Ci₆), or a PEG-distearyloxypropyl (C]₈). The conjugated lipid that prevents aggregation of particles may be from 0 mol % to about 20 mol % or about 2 mol % of the total lipid present in the particle.

In some embodiments, the nucleic acid-lipid particle further includes cholesterol at, e.g., about 10 mol % to about 60 mol % or about 48 mol % of the total lipid present in the particle.

LNP01

In one embodiment, the lipidoid ND98·4HCl (MW 1487) (Formula I), Cholesterol (Sigma-Aldrich), and PEG-Ceramide C 16 (Avanti Polar Lipids) can be used to prepare lipid-siRNA nanoparticles (i.e., LNP01 particles). Stock solutions of each in ethanol can be prepared as follows: ND98, 133 mg/ml; Cholesterol, 25 mg/ml, PEG-Ceramide C16, 100 mg/ml. The ND98, Cholesterol, and PEG-Ceramide C16 stock solutions can then be combined in a, e.g., 42:48:10 molar ratio. The combined lipid solution can be mixed with aqueous siRNA (e.g., in sodium acetate pH 5) such that the final ethanol concentration is about 35-45% and the final sodium acetate concentration is about 100-300 mM. Lipid-siRNA nanoparticles typically form spontaneously upon mixing. Depending on the desired particle size distribution, the resultant nanoparticle mixture can be extruded through a polycarbonate membrane (e.g., 100 nm cut-off) using, for example, a thermobarrel extruder, such as Lipex Extruder (Northern Lipids, Inc). In some cases, the extrusion step can be omitted. Ethanol removal and simultaneous buffer exchange can be accomplished by, for example, dialysis or tangential flow filtration. Buffer can be exchanged with, for example, phosphate buffered saline (PBS) at about pH 7, e.g., about pH 6.9, about pH 7.0, about pH 7.1, about pH 7.2, about pH 7.3, or about pH 7.4.

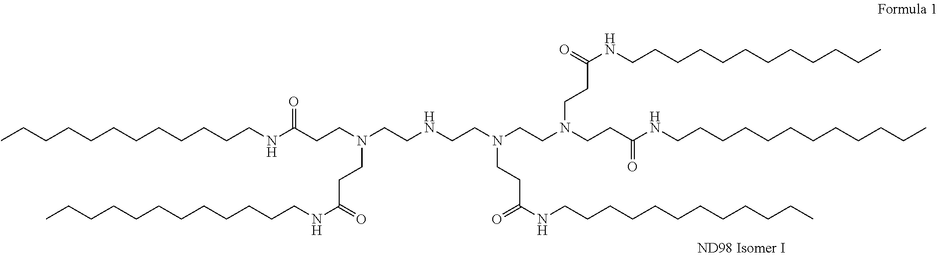

LNP01 formulations are described, e.g., in International Application Publication No. WO 2008/042973, which is hereby incorporated by reference.

Additional exemplary lipid-siRNA formulations are as follows:

| | | cationic lipid/non-cationic | |
|---|---|---|---|
| | | lipid//choleseterol/PEG-lipid conjugate | |
| | Cationic Lipid | Lipid:siRNA ratio | Process |
| LNP05 | 2,2-Dilinoleyl-4- | XTC/DSPC/Cholesterol/PEG-DMG | Extrusion |
| | dimethylaminoethyl- | 57.5/7.5/31.5/3.5 | |
| | [1,3]-dioxolane (XTC) | lipid:siRNA ~6:1 | |
| LNP06 | 2,2-Dilinoleyl-4- | XTC/DSPC/Cholesterol/PEG-DMG | Extrusion |
| | dimethylaminoethyl- | 57.5/7.5/31.5/3.5 | |
| | [1,3]-dioxolane (XTC) | lipid:siRNA ~11:1 | |
| LNP07 | 2,2-Dilinoleyl-4- | XTC/DSPC/Cholesterol/PEG-DMG | In-line |
| | dimethylaminoethyl- | 60/7.5/31/1.5, | mixing |
| | [1,3]-dioxolane (XTC) | lipid:siRNA ~6:1 | |
| LNP08 | 2,2-Dilinoleyl-4- | XTC/DSPC/Cholesterol/PEG-DMG | In-line |
| | dimethylaminoethyl- | 60/7.5/31/1.5, | mixing |
| | [1,3]-dioxolane (XTC) | lipid:siRNA ~11:1 | |
| LNP09 | 2,2-Dilinoleyl-4- | XTC/DSPC/Cholesterol/PEG-DMG | In-line |
| | dimethylaminoethyl- | 50/10/38.5/1.5 | mixing |
| | [1,3]-dioxolane (XTC) | Lipid:siRNA 10:1 | |
| SNALP | 1,2-Dilinolenyloxy- | DLinDMA/DPPC/Cholesterol/PEG-cDMA | |
| | N,N- | (57.1/7.1/34.4/1.4) | |
| | dimethylaminopropane | lipid:siRNA ~7:1 | |
| | (DLinDMA) | | |
| SNALP- | 2,2-Dilinoleyl-4- | XTC/DPPC/Cholesterol/PEG-cDMA | |
| XTC | dimethylaminoethyl- | 57.1/7.1/34.4/1.4 | |
| | [1,3]-dioxolane (XTC) | lipid:siRNA ~7:1 | |

Carriers

Certain compositions of the present invention also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The coadministration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. For example, the recovery of a partially phosphorothioate dsRNA in hepatic tissue can be reduced when it is coadministered with polyinosinic acid, dextran sulfate, polycytidic acid or 4-acetamido-4′ isothiocyano-stilbene-2,2′-disulfonic acid (Miyao et al., DsRNA Res. Dev., 1995, 5, 115-121; Takakura et al., DsRNA & Nucl. Acid Drug Dev., 1996, 6, 177-183.

Excipients

In contrast to a carrier compound, a “pharmaceutical carrier” or “excipient” is a pharmaceutically acceptable solvent, suspending agent or any other pharmacologically inert vehicle for delivering one or more nucleic acids to an animal. The excipient may be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc).

Pharmaceutically acceptable organic or inorganic excipient suitable for non-parenteral administration which do not deleteriously react with nucleic acids can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids may include sterile and non-sterile aqueous solutions, non-aqueous solutions in common solvents such as alcohols, or solutions of the nucleic acids in liquid or solid oil bases. The solutions may also contain buffers, diluents and other suitable additives. Pharmaceutically acceptable organic or inorganic excipients suitable for non-parenteral administration which do not deleteriously react with nucleic acids can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

Other Components

The compositions of the present invention may additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Aqueous suspensions may contain substances which increase the viscosity of the suspension including, for example, sodium carboxymethylcellulose, sorbitol and/or dextran. The suspension may also contain stabilizers.

In some embodiments, compositions featured in the invention include (a) one or more dsRNA compounds and (b) one or more anti-cytokine biologic agents which function by a non-RNAi mechanism. Examples of such biologics include, biologics that target IL1β (e.g., anakinra), IL6 (tocilizumab), or TNF (etanercept, infliximab, adlimumab, or certolizumab).

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred.

The data obtained from cell culture assays and animal studies can be used in formulating a range of dosage for use in humans. The dosage of compositions of the invention lies generally within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range of the compound or, when appropriate, of the polypeptide product of a target sequence (e.g., achieving a decreased concentration of the polypeptide) that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

In addition to their administration, as discussed above, the dsRNAs featured in the invention can be administered in combination with other known agents effective in treatment of pathological processes mediated by SID-1 expression. In any event, the administering physician can adjust the amount and timing of dsRNA administration on the basis of results observed using standard measures of efficacy known in the art or described herein.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

### dsRNA Synthesis

Source of Reagents

Where the source of a reagent is not specifically given herein, such reagent may be obtained from any supplier of reagents for molecular biology at a quality/purity standard for application in molecular biology.

siRNA Synthesis

Single-stranded RNAs were produced by solid phase synthesis on a scale of 1 μmole using an Expedite 8909 synthesizer (Applied Biosystems, Applera Deutschland GmbH, Darmstadt, Germany) and controlled pore glass (CPG, 500 Å, Proligo Biochemie GmbH, Hamburg, Germany) as solid support. RNA and RNA containing 2′-O-methyl nucleotides were generated by solid phase synthesis employing the corresponding phosphoramidites and 2′-O-methyl phosphoramidites, respectively (Proligo Biochemie GmbH, Hamburg, Germany). These building blocks were incorporated at selected sites within the sequence of the oligoribonucleotide chain using standard nucleoside phosphoramidite chemistry such as described in Current protocols in nucleic acid chemistry, Beaucage, S. L. et al. (Edrs.), John Wiley & Sons, Inc., New York, N.Y., USA. Phosphorothioate linkages were introduced by replacement of the iodine oxidizer solution with a solution of the Beaucage reagent (Chruachem Ltd, Glasgow, UK) in acetonitrile (1%). Further ancillary reagents were obtained from Mallinckrodt Baker (Griesheim, Germany).

Deprotection and purification of the crude oligoribonucleotides by anion exchange HPLC were carried out according to established procedures. Yields and concentrations were determined by UV absorption of a solution of the respective RNA at a wavelength of 260 nm using a spectral photometer (DU 640B, Beckman Coulter GmbH, Unterschleiβheim, Germany). Double stranded RNA was generated by mixing an equimolar solution of complementary strands in annealing buffer (20 mM sodium phosphate, pH 6.8; 100 mM sodium chloride), heated in a water bath at 85-90° C. for 3 minutes and cooled to room temperature over a period of 3-4 hours. The annealed RNA solution was stored at −20° C. until use.

For the synthesis of 3′-cholesterol-conjugated siRNAs (herein referred to as -Chol-3), an appropriately modified solid support was used for RNA synthesis. The modified solid support was prepared as follows:

### Diethyl-2-azabutane-1,4-dicarboxylate AA

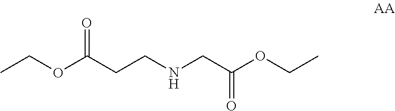

A 4.7 M aqueous solution of sodium hydroxide (50 mL) was added into a stirred, ice-cooled solution of ethyl glycinate hydrochloride (32.19 g, 0.23 mole) in water (50 mL). Then, ethyl acrylate (23.1 g, 0.23 mole) was added and the mixture was stirred at room temperature until completion of the reaction was ascertained by TLC. After 19 h the solution was partitioned with dichloromethane (3×100 mL). The organic layer was dried with anhydrous sodium sulfate, filtered and evaporated. The residue was distilled to afford AA (28.8 g, 61%).

### 3-{Ethoxycarbonylmethyl-[6-(9H-fluoren-9-ylmethoxycarbonyl-amino)-hexanoyl]-amino}-propionic acid ethyl ester AB

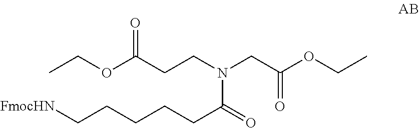

Fmoc-6-amino-hexanoic acid (9.12 g, 25.83 mmol) was dissolved in dichloromethane (50 mL) and cooled with ice. Diisopropylcarbodiimde (3.25 g, 3.99 mL, 25.83 mmol) was added to the solution at 0° C. It was then followed by the addition of Diethyl-azabutane-1,4-dicarboxylate (5 g, 24.6 mmol) and dimethylamino pyridine (0.305 g, 2.5 mmol). The solution was brought to room temperature and stirred further for 6 h. Completion of the reaction was ascertained by TLC. The reaction mixture was concentrated under vacuum and ethyl acetate was added to precipitate diisopropyl urea. The suspension was filtered. The filtrate was washed with 5% aqueous hydrochloric acid, 5% sodium bicarbonate and water. The combined organic layer was dried over sodium sulfate and concentrated to give the crude product which was purified by column chromatography (50% EtOAC/Hexanes) to yield 11.87 g (88%) of AB.

### 3-[(6-Amino-hexanoyl)-ethoxycarbonylmethyl-amino]-propionic acid ethyl ester AC

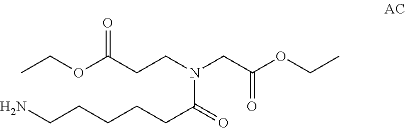

3-{Ethoxycarbonylmethyl-[6-(9H-fluoren-9-ylmethoxycarbonylamino)-hexanoyl]-amino}-propionic acid ethyl ester AB (11.5 g, 21.3 mmol) was dissolved in 20% piperidine in dimethylformamide at 0° C. The solution was continued stirring for 1 h. The reaction mixture was concentrated under vacuum, water was added to the residue, and the product was extracted with ethyl acetate. The crude product was purified by conversion into its hydrochloride salt.

### 3-({6-[17-(1,5-Dimethyl-hexyl)-10,13-dimethyl-2,3,4,7,8,9,10,11,12,13,14,15,16,17-tetradecahydro-1H-cyclopenta[a]phenanthren-3-yloxycarbonylamino]-hexanoyl}ethoxycarbonylmethyl-amino)-propionic acid ethyl ester AD

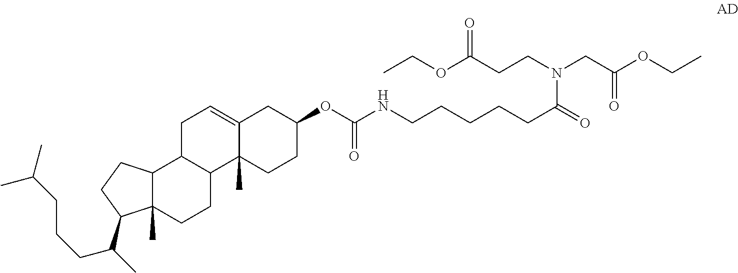

The hydrochloride salt of 3-[(6-Amino-hexanoyl)-ethoxycarbonylmethyl-amino]-propionic acid ethyl ester AC (4.7 g, 14.8 mmol) was taken up in dichloromethane. The suspension was cooled to 0° C. on ice. To the suspension diisopropylethylamine (3.87 g, 5.2 mL, 30 mmol) was added. To the resulting solution cholesteryl chloroformate (6.675 g, 14.8 mmol) was added. The reaction mixture was stirred overnight. The reaction mixture was diluted with dichloromethane and washed with 10% hydrochloric acid. The product was purified by flash chromatography (10.3 g, 92%).

### 1-{6-[17-(1,5-Dimethyl-hexyl)-10,13-dimethyl-2,3,4,7,8,9,10,11,12,13,14,15,16,17-tetradecahydro-1H-cyclopenta[a]phenanthren-3-yloxycarbonylamino]-hexanoyl}-4-oxo-pyrrolidine-3-carboxylic acid ethyl ester AE

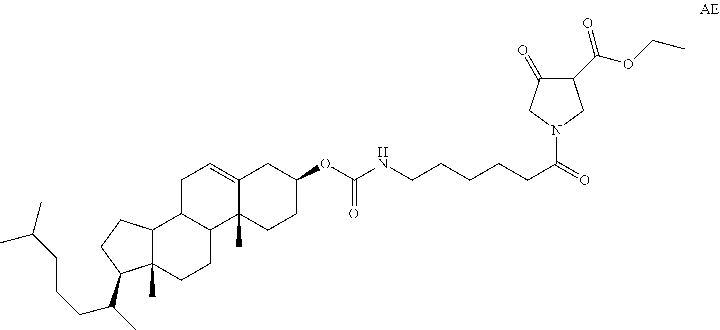

Potassium t-butoxide (1.1 g, 9.8 mmol) was slurried in 30 mL of dry toluene. The mixture was cooled to 0° C. on ice and 5 g (6.6 mmol) of diester AD was added slowly with stirring within 20 mins. The temperature was kept below 5° C. during the addition. The stirring was continued for 30 mins at 0° C. and 1 mL of glacial acetic acid was added, immediately followed by 4 g of NaH₂PO₄.H₂O in 40 mL of water The resultant mixture was extracted twice with 100 mL of dichloromethane each and the combined organic extracts were washed twice with 10 mL of phosphate buffer each, dried, and evaporated to dryness. The residue was dissolved in 60 mL of toluene, cooled to 0° C. and extracted with three 50 mL portions of cold pH 9.5 carbonate buffer. The aqueous extracts were adjusted to pH 3 with phosphoric acid, and extracted with five 40 mL portions of chloroform which were combined, dried and evaporated to dryness. The residue was purified by column chromatography using 25% ethylacetate/hexane to afford 1.9 g of b-ketoester (39%).

### [6-(3-Hydroxy-4-hydroxymethyl-pyrrolidin-1-yl)-6-oxo-hexyl]-carbamic acid 17-(1,5-dimethyl-hexyl)-10,13-dimethyl-2,3,4,7,8,9,10,11,12,13,14,15,16,17-tetradecahydro-1H-cyclopenta[a]phenanthren-3-yl ester AF

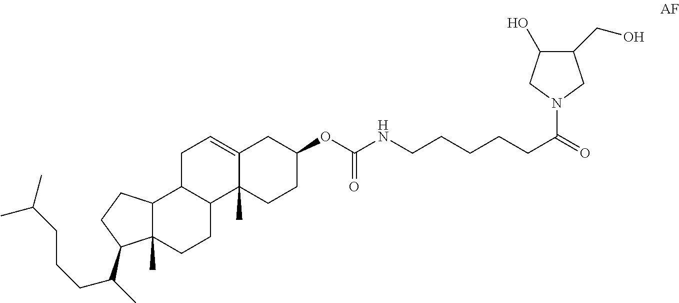

Methanol (2 mL) was added dropwise over a period of 1 h to a refluxing mixture of b-ketoester AE (1.5 g, 2.2 mmol) and sodium borohydride (0.226 g, 6 mmol) in tetrahydrofuran (10 mL). Stirring was continued at reflux temperature for 1 h. After cooling to room temperature, 1 N HCl (12.5 mL) was added, the mixture was extracted with ethylacetate (3×40 mL) 1he combined ethylacetate layer was dried over anhydrous sodium sulfate and concentrated under vacuum to yield the product which was purified by column chromatography (10% MeOH/CHCl₃) (89%).

### (6-{3-[Bis-(4-methoxy-phenyl)-phenyl-methoxymethyl]-4-hydroxy-pyrrolidin-1-yl}-6-oxo-hexyl)-carbamic acid 17-(1,5-dimethyl-hexyl)-10,13-dimethyl-2,3,4,7,8,9,10,11,12,13,14,15,16,17-tetradecahydro-1H-cyclopenta[a]phenanthren-3-yl ester AG

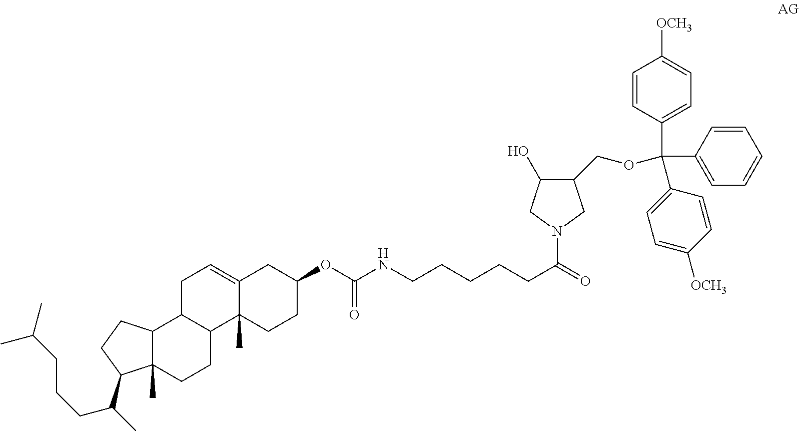

Diol AF (1.25 gm 1.994 mmol) was dried by evaporating with pyridine (2×5 mL) in vacuo. Anhydrous pyridine (10 mL) and 4,4′-dimethoxytritylchloride (0.724 g, 2.13 mmol) were added with stirring. The reaction was carried out at room temperature overnight. The reaction was quenched by the addition of methanol. The reaction mixture was concentrated under vacuum and to the residue dichloromethane (50 mL) was added. The organic layer was washed with 1M aqueous sodium bicarbonate. The organic layer was dried over anhydrous sodium sulfate, filtered and concentrated. The residual pyridine was removed by evaporating with toluene. The crude product was purified by column chromatography (2% MeOH/Chloroform, Rf=0.5 in 5% MeOH/CHCl₃) (1.75 g, 95%).

### Succinic acid mono-(4-[bis-(4-methoxy-phenyl)-phenyl-methoxymethyl]-1-{6-[17-(1,5-dimethyl-hexyl)-10,13-dimethyl 2,3,4,7,8,9,10,11,12,13,14,15,16,17-tetradecahydro-1H cyclopenta[a]phenanthren-3-yloxycarbonylamino]-hexanoyl}-pyrrolidin-3-yl)ester AH

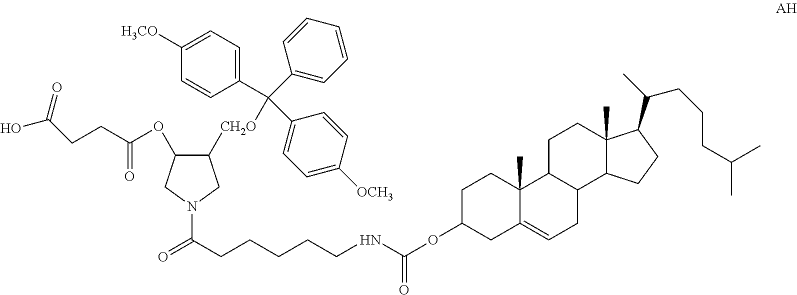

Compound AG (1.0 g, 1.05 mmol) was mixed with succinic anhydride (0.150 g, 1.5 mmol) and DMAP (0.073 g, 0.6 mmol) and dried in a vacuum at 40° C. overnight. The mixture was dissolved in anhydrous dichloroethane (3 mL), triethylamine (0.318 g, 0.440 mL, 3.15 mmol) was added and the solution was stirred at room temperature under argon atmosphere for 16 h. It was then diluted with dichloromethane (40 mL) and washed with ice cold aqueous citric acid (5 wt %, 30 mL) and water (2×20 mL). The organic phase was dried over anhydrous sodium sulfate and concentrated to dryness. The residue was used as such for the next step.

Cholesterol Derivatised CPG AI

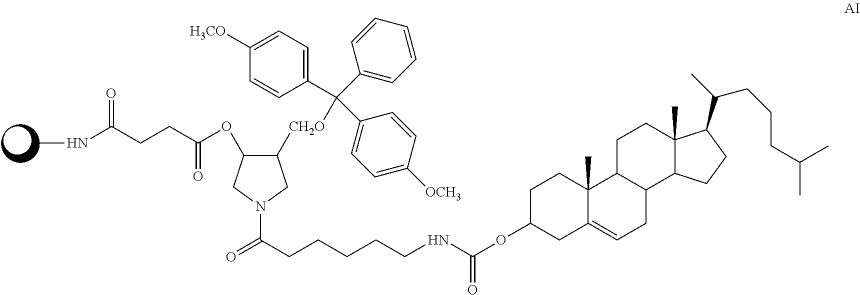

Succinate AH (0.254 g, 0.242 mmol) was dissolved in a mixture of dichloromethane/acetonitrile (3:2, 3 mL). To that solution DMAP (0.0296 g, 0.242 mmol) in acetonitrile (1.25 mL), 2,2′-Dithio-bis(5-nitropyridine) (0.075 g, 0.242 mmol) in acetonitrile/dichloroethane (3:1, 1.25 mL) were added successively. To the resulting solution triphenylphosphine (0.064 g, 0.242 mmol) in acetonitrile (0.6 ml) was added. The reaction mixture turned bright orange in color. The solution was agitated briefly using a wrist-action shaker (5 mins). Long chain alkyl amine-CPG (LCAA-CPG) (1.5 g, 61 mM) was added. The suspension was agitated for 2 h. The CPG was filtered through a sintered funnel and washed with acetonitrile, dichloromethane and ether successively. Unreacted amino groups were masked using acetic anhydride/pyridine. The achieved loading of the CPG was measured by taking UV measurement (37 mM/g).

The synthesis of siRNAs bearing a 5′-12-dodecanoic acid bisdecylamide group (herein referred to as “5′-C32-”) or a 5′-cholesteryl derivative group (herein referred to as “5′-Chol-”) was performed as described in WO 2004/065601, except that, for the cholesteryl derivative, the oxidation step was performed using the Beaucage reagent in order to introduce a phosphorothioate linkage at the 5′-end of the nucleic acid oligomer.

Nucleic acid sequences are represented below using standard nomenclature, and specifically the abbreviations of Table 1.

TABLE 1
Abbreviations of nucleotide monomers used in nucleic
acid sequence representation. It will be understood that
these monomers, when present in an oligonucleotide, are
mutually linked by 5′-3′-phosphodiester bonds.

| Abbreviation | Nucleotide(s) |
|---|---|
| A | adenosine-3′-phosphate |
| C | cytidine-3′-phosphate |
| G | guanosine-3′-phosphate |
| U | uridine-3′-phosphate |
| c | 2′-O-methylcytidine-3′-phosphate |
| dT | 2′-deoxythymidine-3′-phosphate |
| dTs | 2′-deoxythymidine-3′-phosphorothioate |
| u | 2′-O-methyluridine-3′-phosphate |

### Example 2

### SID-1 siRNA Design

Transcripts

siRNA design was carried out to identify siRNAs targeting the gene SID1 transmembrane family, member 1 from human (symbol “SIDT1”), mouse (symbol “Sidt1”) and rat (symbol “Sidt1”). The design used the SIDT1 transcripts NM_{—}017699.2 (human), NM_{—}198034.2 (mouse), and XM_{—}221455.4 (rat) from the NCBI Refseq collection.

A total of 101 siRNA sense oligos with 100% identity to the transcripts from all three SIDT1 genes were discovered.

siRNA Design and Specificity Prediction

Target specificity was predicted for each sense and antisense sequence. The SIDT1 siRNAs were used in a comprehensive search against the human, mouse and rat transcriptomes (defined as the set of NM_{—} and XM_{—} records within the NCBI Refseq set) using the FASTA algorithm. The Python script ‘offtargetFasta.py’ was then used to parse the alignments and generate a score based on the position and number of mismatches between the siRNA and any potential ‘off-target’ transcript. The off-target score is weighted to emphasize differences in the ‘seed’ region of siRNAs, in positions 2-9 from the 5′ end of the molecule. The off-target score is calculated as follows: mismatches between the oligo and the transcript are given penalties. A mismatch in the seed region in positions 2-9 of the oligo is given a penalty of 2.8; mismatches in the putative cleavage sites 10 and 11 are given a penalty of 1.2, and all other mismatches a penalty of 1. The off-target score for each oligo-transcript pair is then calculated by summing the mismatch penalties. The lowest off-target score from all the oligo-transcript pairs is then determined and used in subsequent sorting of oligos. Both siRNA strands were assigned to a category of specificity according to the calculated scores: a score above 3 qualifies as highly specific; equal to 3 qualifies as specific; and between 2.2 and 2.8 qualifies as moderately specific. In picking which oligos to synthesize, the off-target score of the antisense strand was sorted from high to low.

siRNA Sequence Selection

A total of 43 sense and 43 antisense rat SIDT1 derived siRNA oligos were synthesized and formed into 43 duplexes. The oligos are presented in Tables 2-4 (human SIDT1). In Tables 3A, 3B, 4A and 4B, upper case letters represent ribonucleosides and lower case letters represent 2′ O-Methyl (2′OMe) nucleosides. The phosphodiester linkage is represented as “dTdT” and the phosphorothioate linkage is represented as “dTsdT.” The sense and antisense single strands are represented as “S” and “AS” respectively.

TABLE 2
Identification numbers for human SIDT1 dsRNAs

| Duplex # | Sense Oligo # | Antisense Oligo # |
|---|---|---|
| AD-3891 | A-31481 | A-31482 |
| AD-3892 | A-31483 | A-31484 |
| AD-3893 | A-31485 | A-31486 |
| AD-3894 | A-31487 | A-31488 |
| AD-3895 | A-31489 | A-31490 |
| AD-3896 | A-31491 | A-31492 |
| AD-3897 | A-31493 | A-31494 |
| AD-3898 | A-31495 | A-31496 |
| AD-3899 | A-31497 | A-31498 |
| AD-3900 | A-31499 | A-31500 |
| AD-3901 | A-31501 | A-31502 |
| AD-3902 | A-31503 | A-31504 |
| AD-3903 | A-31505 | A-31506 |
| AD-3904 | A-31507 | A-31508 |
| AD-3905 | A-31509 | A-31510 |
| AD-3906 | A-31511 | A-31512 |
| AD-3907 | A-31513 | A-31514 |
| AD-3908 | A-31515 | A-31516 |
| AD-3909 | A-31517 | A-31518 |
| AD-3910 | A-31519 | A-31520 |
| AD-3911 | A-31521 | A-31522 |
| AD-3912 | A-31523 | A-31524 |
| AD-3945 | A-31027 | A-31028 |
| AD-3946 | A-31029 | A-31030 |
| AD-3947 | A-31031 | A-31032 |
| AD-3948 | A-31033 | A-31034 |
| AD-3949 | A-31035 | A-31036 |
| AD-3950 | A-31037 | A-31038 |
| AD-3951 | A-31039 | A-31040 |
| AD-3952 | A-31041 | A-31042 |
| AD-3953 | A-31043 | A-31044 |
| AD-3954 | A-31045 | A-31046 |
| AD-3955 | A-31047 | A-31048 |
| AD-3956 | A-31049 | A-31050 |
| AD-3957 | A-31051 | A-31052 |
| AD-3958 | A-31053 | A-31054 |
| AD-3959 | A-31055 | A-31056 |
| AD-3960 | A-31057 | A-31058 |
| AD-3961 | A-31059 | A-31060 |
| AD-3962 | A-31061 | A-31062 |
| AD-3963 | A-31063 | A-31064 |
| AD-3964 | A-31065 | A-31066 |
| AD-3965 | A-31067 | A-31068 |
| AD-3966 | A-31069 | A-31070 |

TABLE 3A
Sense and antisense strand sequences of human SIDT1 dsRNAs

| Strand ID | | | | | |
|---|---|---|---|---|---|
| (s = | Position | | | Sense strand sequence | |
| sense; | of 5' base | | SEQ | with | SEQ |
| as = | on | | ID | 3' dinucleotide | ID |
| antisense) | transcript | Sequence (5′ to 3′) | NO: | overhang (5' to 3') | NO: |
| s | 2336 | ACCAUGUCUGCCCUAAUUA | 1 | ACCAUGUCUGCCCUAAUUANN | 87 |
| as | 2354 | UAAUUAGGGCAGACAUGGU | 2 | UAAUUAGGGCAGACAUGGUNN | 88 |
| s | 1206 | ACCUCAGUAUUUUCUAUAC | 3 | ACCUCAGUAUUUUCUAUACNN | 89 |
| as | 1224 | GUAUAGAAAAUACUGAGGU | 4 | GUAUAGAAAAUACUGAGGUNN | 90 |
| s | 1200 | CUCUCAACCUCAGUAUUUU | 5 | CUCUCAACCUCAGUAUUUUNN | 91 |
| as | 1218 | AAAAUACUGAGGUUGAGAG | 6 | AAAAUACUGAGGUUGAGAGNN | 92 |
| s | 1356 | UGUCUAUCAGUCCAUGACC | 7 | UGUCUAUCAGUCCAUGACCNN | 93 |
| as | 1374 | GGUCAUGGACUGAUAGACA | 8 | GGUCAUGGACUGAUAGACANN | 94 |
| s | 1902 | CAUCCGGACCAAGAUGUUC | 9 | CAUCCGGACCAAGAUGUUCNN | 95 |
| as | 1920 | GAACAUCUUGGUCCGGAUG | 10 | GAACAUCUUGGUCCGGAUGNN | 96 |
| s | 1201 | UCUCAACCUCAGUAUUUUC | 11 | UCUCAACCUCAGUAUUUUCNN | 97 |
| as | 1219 | GAAAAUACUGAGGUUGAGA | 12 | GAAAAUACUGAGGUUGAGANN | 98 |
| s | 1901 | UCAUCCGGACCAAGAUGUU | 13 | UCAUCCGGACCAAGAUGUUNN | 99 |
| as | 1919 | AACAUCUUGGUCCGGAUGA | 14 | AACAUCUUGGUCCGGAUGANN | 100 |
| s | 1351 | AAUGGUGUCUAUCAGUCCA | 15 | AAUGGUGUCUAUCAGUCCANN | 101 |
| as | 1369 | UGGACUGAUAGACACCAUU | 16 | UGGACUGAUAGACACCAUUNN | 102 |
| s | 1207 | CCUCAGUAUUUUCUAUACA | 17 | CCUCAGUAUUUUCUAUACANN | 103 |
| as | 1225 | UGUAUAGAAAAUACUGAGG | 18 | UGUAUAGAAAAUACUGAGGNN | 104 |
| s | 2273 | ACUUUGGUCUCUUCUACGC | 19 | ACUUUGGUCUCUUCUACGCNN | 105 |
| as | 2291 | GCGUAGAAGAGACCAAAGU | 20 | GCGUAGAAGAGACCAAAGUNN | 106 |
| s | 1198 | CCCUCUCAACCUCAGUAUU | 21 | CCCUCUCAACCUCAGUAUUNN | 107 |
| as | 1216 | AAUACUGAGGUUGAGAGGG | 22 | AAUACUGAGGUUGAGAGGGNN | 108 |
| s | 2267 | CCAAACACUUUGGUCUCUU | 23 | CCAAACACUUUGGUCUCUUNN | 109 |
| as | 2285 | AAGAGACCAAAGUGUUUGG | 24 | AAGAGACCAAAGUGUUUGGNN | 110 |
| s | 1205 | AACCUCAGUAUUUUCUAUA | 25 | AACCUCAGUAUUUUCUAUANN | 111 |
| as | 1223 | UAUAGAAAAUACUGAGGUU | 26 | UAUAGAAAAUACUGAGGUUNN | 112 |
| s | 1357 | GUCUAUCAGUCCAUGACCA | 27 | GUCUAUCAGUCCAUGACCANN | 113 |
| as | 1375 | UGGUCAUGGACUGAUAGAC | 28 | UGGUCAUGGACUGAUAGACNN | 114 |
| s | 824 | UCAGCACCGAGAACAUCUA | 29 | UCAGCACCGAGAACAUCUANN | 115 |
| as | 842 | UAGAUGUUCUCGGUGCUGA | 30 | UAGAUGUUCUCGGUGCUGANN | 116 |
| s | 1354 | GGUGUCUAUCAGUCCAUGA | 31 | GGUGUCUAUCAGUCCAUGANN | 117 |
| as | 1372 | UCAUGGACUGAUAGACACC | 32 | UCAUGGACUGAUAGACACCNN | 118 |
| s | 1197 | CCCCUCUCAACCUCAGUAU | 33 | CCCCUCUCAACCUCAGUAUNN | 119 |
| as | 1215 | AUACUGAGGUUGAGAGGGG | 34 | AUACUGAGGUUGAGAGGGGNN | 120 |
| s | 826 | AGCACCGAGAACAUCUACU | 35 | AGCACCGAGAACAUCUACUNN | 121 |
| as | 844 | AGUAGAUGUUCUCGGUGCU | 36 | AGUAGAUGUUCUCGGUGCUNN | 122 |
| s | 825 | CAGCACCGAGAACAUCUAC | 37 | CAGCACCGAGAACAUCUACNN | 123 |
| as | 843 | GUAGAUGUUCUCGGUGCUG | 38 | GUAGAUGUUCUCGGUGCUGNN | 124 |
| s | 1352 | AUGGUGUCUAUCAGUCCAU | 39 | AUGGUGUCUAUCAGUCCAUNN | 125 |
| as | 1370 | AUGGACUGAUAGACACCAU | 40 | AUGGACUGAUAGACACCAUNN | 126 |
| s | 2270 | AACACUUUGGUCUCUUCUA | 41 | AACACUUUGGUCUCUUCUANN | 127 |
| as | 2288 | UAGAAGAGACCAAAGUGUU | 42 | UAGAAGAGACCAAAGUGUUNN | 128 |
| s | 3112 | AGAGACCAGAUCCCUGUCU | 43 | AGAGACCAGAUCCCUGUCUNN | 129 |
| as | 3130 | AGACAGGGAUCUGGUCUCU | 44 | AGACAGGGAUCUGGUCUCUNN | 130 |
| s | 2336 | ACCAUGUCUGCCCUAAUUA | 45 | ACCAUGUCUGCCCUAAUUANN | 131 |
| as | 2354 | UAAUUAGGGCAGACAUGGU | 46 | UAAUUAGGGCAGACAUGGUNN | 132 |
| s | 1206 | ACCUCAGUAUUUUCUAUAC | 47 | ACCUCAGUAUUUUCUAUACNN | 133 |
| as | 1224 | GUAUAGAAAAUACUGAGGU | 48 | GUAUAGAAAAUACUGAGGUNN | 134 |
| s | 1356 | UGUCUAUCAGUCCAUGACC | 49 | UGUCUAUCAGUCCAUGACCNN | 135 |
| as | 1374 | GGUCAUGGACUGAUAGACA | 50 | GGUCAUGGACUGAUAGACANN | 136 |
| s | 1902 | CAUCCGGACCAAGAUGUUC | 51 | CAUCCGGACCAAGAUGUUCNN | 137 |
| as | 1920 | GAACAUCUUGGUCCGGAUG | 52 | GAACAUCUUGGUCCGGAUGNN | 138 |
| s | 1201 | UCUCAACCUCAGUAUUUUC | 53 | UCUCAACCUCAGUAUUUUCNN | 139 |
| as | 1219 | GAAAAUACUGAGGUUGAGA | 54 | GAAAAUACUGAGGUUGAGANN | 140 |
| s | 1901 | UCAUCCGGACCAAGAUGUU | 55 | UCAUCCGGACCAAGAUGUUNN | 141 |
| as | 1919 | AACAUCUUGGUCCGGAUGA | 56 | AACAUCUUGGUCCGGAUGANN | 142 |
| s | 1351 | AAUGGUGUCUAUCAGUCCA | 57 | AAUGGUGUCUAUCAGUCCANN | 143 |
| as | 1369 | UGGACUGAUAGACACCAUU | 58 | UGGACUGAUAGACACCAUUNN | 144 |
| s | 1207 | CCUCAGUAUUUUCUAUACA | 59 | CCUCAGUAUUUUCUAUACANN | 145 |
| as | 1225 | UGUAUAGAAAAUACUGAGG | 60 | UGUAUAGAAAAUACUGAGGNN | 146 |
| s | 2273 | ACUUUGGUCUCUUCUACGC | 61 | ACUUUGGUCUCUUCUACGCNN | 147 |
| as | 2291 | GCGUAGAAGAGACCAAAGU | 62 | GCGUAGAAGAGACCAAAGUNN | 148 |
| s | 1198 | CCCUCUCAACCUCAGUAUU | 63 | CCCUCUCAACCUCAGUAUUNN | 149 |
| as | 1216 | AAUACUGAGGUUGAGAGGG | 64 | AAUACUGAGGUUGAGAGGGNN | 150 |
| s | 2267 | CCAAACACUUUGGUCUCUU | 65 | CCAAACACUUUGGUCUCUUNN | 151 |
| as | 2285 | AAGAGACCAAAGUGUUUGG | 66 | AAGAGACCAAAGUGUUUGGNN | 152 |
| s | 1205 | AACCUCAGUAUUUUCUAUA | 67 | AACCUCAGUAUUUUCUAUANN | 153 |
| as | 1223 | UAUAGAAAAUACUGAGGUU | 68 | UAUAGAAAAUACUGAGGUUNN | 154 |
| s | 1357 | GUCUAUCAGUCCAUGACCA | 69 | GUCUAUCAGUCCAUGACCANN | 155 |
| as | 1375 | UGGUCAUGGACUGAUAGAC | 70 | UGGUCAUGGACUGAUAGACNN | 156 |
| s | 824 | UCAGCACCGAGAACAUCUA | 71 | UCAGCACCGAGAACAUCUANN | 157 |
| as | 842 | UAGAUGUUCUCGGUGCUGA | 72 | UAGAUGUUCUCGGUGCUGANN | 158 |
| s | 1354 | GGUGUCUAUCAGUCCAUGA | 73 | GGUGUCUAUCAGUCCAUGANN | 159 |
| as | 1372 | UCAUGGACUGAUAGACACC | 74 | UCAUGGACUGAUAGACACCNN | 160 |
| s | 1197 | CCCCUCUCAACCUCAGUAU | 75 | CCCCUCUCAACCUCAGUAUNN | 161 |
| as | 1215 | AUACUGAGGUUGAGAGGGG | 76 | AUACUGAGGUUGAGAGGGGNN | 162 |
| s | 826 | AGCACCGAGAACAUCUACU | 77 | AGCACCGAGAACAUCUACUNN | 163 |
| as | 844 | AGUAGAUGUUCUCGGUGCU | 78 | AGUAGAUGUUCUCGGUGCUNN | 164 |
| s | 825 | CAGCACCGAGAACAUCUAC | 79 | CAGCACCGAGAACAUCUACNN | 165 |
| as | 843 | GUAGAUGUUCUCGGUGCUG | 80 | GUAGAUGUUCUCGGUGCUGNN | 166 |
| s | 1352 | AUGGUGUCUAUCAGUCCAU | 81 | AUGGUGUCUAUCAGUCCAUNN | 167 |
| as | 1370 | AUGGACUGAUAGACACCAU | 82 | AUGGACUGAUAGACACCAUNN | 168 |
| s | 2270 | AACACUUUGGUCUCUUCUA | 83 | AACACUUUGGUCUCUUCUANN | 169 |
| as | 2288 | UAGAAGAGACCAAAGUGUU | 84 | UAGAAGAGACCAAAGUGUUNN | 170 |
| s | 3112 | AGAGACCAGAUCCCUGUCU | 85 | AGAGACCAGAUCCCUGUCUNN | 171 |
| as | 3130 | AGACAGGGAUCUGGUCUCU | 86 | AGACAGGGAUCUGGUCUCUNN | 172 |

TABLE 3B
Sense and antisense strand sequences of human SIDT1 dsRNAs

| | | Sequence with | | Sequence with | |
|---|---|---|---|---|---|
| | Position | 3′deoxythimidine | | 3′ deoxythimidine | |
| | of 5′ | overhang | | overhang | |
| | base on | (phosphodiester | SEQ | (phosphorothioate | SEQ |
| Strand | tran- | linkage) | ID | linkage) | ID |
| ID | script | (5′ to 3′) | NO: | (5′ to 3′) | NO: |
| s | 2336 | ACCAUGUCUGCCCUAAUUAdTdT | 173 | ACCAUGUCUGCCCUAAUUAdTsdT | 259 |
| as | 2354 | UAAUUAGGGCAGACAUGGUdTdT | 174 | UAAUUAGGGCAGACAUGGUdTsdT | 260 |
| s | 1206 | ACCUCAGUAUUUUCUAUACdTdT | 175 | ACCUCAGUAUUUUCUAUACdTsdT | 261 |
| as | 1224 | GUAUAGAAAAUACUGAGGUdTdT | 176 | GUAUAGAAAAUACUGAGGUdTsdT | 262 |
| s | 1200 | CUCUCAACCUCAGUAUUUUdTdT | 177 | CUCUCAACCUCAGUAUUUUdTsdT | 263 |
| as | 1218 | AAAAUACUGAGGUUGAGAGdTdT | 178 | AAAAUACUGAGGUUGAGAGdTsdT | 264 |
| s | 1356 | UGUCUAUCAGUCCAUGACCdTdT | 179 | UGUCUAUCAGUCCAUGACCdTsdT | 265 |
| as | 1374 | GGUCAUGGACUGAUAGACAdTdT | 180 | GGUCAUGGACUGAUAGACAdTsdT | 266 |
| s | 1902 | CAUCCGGACCAAGAUGUUCdTdT | 181 | CAUCCGGACCAAGAUGUUCdTsdT | 267 |
| as | 1920 | GAACAUCUUGGUCCGGAUGdTdT | 182 | GAACAUCUUGGUCCGGAUGdTsdT | 268 |
| s | 1201 | UCUCAACCUCAGUAUUUUCdTdT | 183 | UCUCAACCUCAGUAUUUUCdTsdT | 269 |
| as | 1219 | GAAAAUACUGAGGUUGAGAdTdT | 184 | GAAAAUACUGAGGUUGAGAdTsdT | 270 |
| s | 1901 | UCAUCCGGACCAAGAUGUUdTdT | 185 | UCAUCCGGACCAAGAUGUUdTsdT | 271 |
| as | 1919 | AACAUCUUGGUCCGGAUGAdTdT | 186 | AACAUCUUGGUCCGGAUGAdTsdT | 272 |
| s | 1351 | AAUGGUGUCUAUCAGUCCAdTdT | 187 | AAUGGUGUCUAUCAGUCCAdTsdT | 273 |
| as | 1369 | UGGACUGAUAGACACCAUUdTdT | 188 | UGGACUGAUAGACACCAUUdTsdT | 274 |
| s | 1207 | CCUCAGUAUUUUCUAUACAdTdT | 189 | CCUCAGUAUUUUCUAUACAdTsdT | 275 |
| as | 1225 | UGUAUAGAAAAUACUGAGGdTdT | 190 | UGUAUAGAAAAUACUGAGGdTsdT | 276 |
| s | 2273 | ACUUUGGUCUCUUCUACGCdTdT | 191 | ACUUUGGUCUCUUCUACGCdTsdT | 277 |
| as | 2291 | GCGUAGAAGAGACCAAAGUdTdT | 192 | GCGUAGAAGAGACCAAAGUdTsdT | 278 |
| s | 1198 | CCCUCUCAACCUCAGUAUUdTdT | 193 | CCCUCUCAACCUCAGUAUUdTsdT | 279 |
| as | 1216 | AAUACUGAGGUUGAGAGGGdTdT | 194 | AAUACUGAGGUUGAGAGGGdTsdT | 280 |
| s | 2267 | CCAAACACUUUGGUCUCUUdTdT | 195 | CCAAACACUUUGGUCUCUUdTsdT | 281 |
| as | 2285 | AAGAGACCAAAGUGUUUGGdTdT | 196 | AAGAGACCAAAGUGUUUGGdTsdT | 282 |
| s | 1205 | AACCUCAGUAUUUUCUAUAdTdT | 197 | AACCUCAGUAUUUUCUAUAdTsdT | 283 |
| as | 1223 | UAUAGAAAAUACUGAGGUUdTdT | 198 | UAUAGAAAAUACUGAGGUUdTsdT | 284 |
| s | 1357 | GUCUAUCAGUCCAUGACCAdTdT | 199 | GUCUAUCAGUCCAUGACCAdTsdT | 285 |
| as | 1375 | UGGUCAUGGACUGAUAGACdTdT | 200 | UGGUCAUGGACUGAUAGACdTsdT | 286 |
| s | 824 | UCAGCACCGAGAACAUCUAdTdT | 201 | UCAGCACCGAGAACAUCUAdTsdT | 287 |
| as | 842 | UAGAUGUUCUCGGUGCUGAdTdT | 202 | UAGAUGUUCUCGGUGCUGAdTsdT | 288 |
| s | 1354 | GGUGUCUAUCAGUCCAUGAdTdT | 203 | GGUGUCUAUCAGUCCAUGAdTsdT | 289 |
| as | 1372 | UCAUGGACUGAUAGACACCdTdT | 204 | UCAUGGACUGAUAGACACCdTsdT | 290 |
| s | 1197 | CCCCUCUCAACCUCAGUAUdTdT | 205 | CCCCUCUCAACCUCAGUAUdTsdT | 291 |
| as | 1215 | AUACUGAGGUUGAGAGGGGdTdT | 206 | AUACUGAGGUUGAGAGGGGdTsdT | 292 |
| s | 826 | AGCACCGAGAACAUCUACUdTdT | 207 | AGCACCGAGAACAUCUACUdTsdT | 293 |
| as | 844 | AGUAGAUGUUCUCGGUGCUdTdT | 208 | AGUAGAUGUUCUCGGUGCUdTsdT | 294 |
| s | 825 | CAGCACCGAGAACAUCUACdTdT | 209 | CAGCACCGAGAACAUCUACdTsdT | 295 |
| as | 843 | GUAGAUGUUCUCGGUGCUGdTdT | 210 | GUAGAUGUUCUCGGUGCUGdTsdT | 296 |
| s | 1352 | AUGGUGUCUAUCAGUCCAUdTdT | 211 | AUGGUGUCUAUCAGUCCAUdTsdT | 297 |
| as | 1370 | AUGGACUGAUAGACACCAUdTdT | 212 | AUGGACUGAUAGACACCAUdTsdT | 298 |
| s | 2270 | AACACUUUGGUCUCUUCUAdTdT | 213 | AACACUUUGGUCUCUUCUAdTsdT | 299 |
| as | 2288 | UAGAAGAGACCAAAGUGUUdTdT | 214 | UAGAAGAGACCAAAGUGUUdTsdT | 300 |
| s | 3112 | AGAGACCAGAUCCCUGUCUdTdT | 215 | AGAGACCAGAUCCCUGUCUdTsdT | 301 |
| as | 3130 | AGACAGGGAUCUGGUCUCUdTdT | 216 | AGACAGGGAUCUGGUCUCUdTsdT | 302 |
| s | 2336 | ACCAUGUCUGCCCUAAUUAdTdT | 217 | ACCAUGUCUGCCCUAAUUAdTsdT | 303 |
| as | 2354 | UAAUUAGGGCAGACAUGGUdTdT | 218 | UAAUUAGGGCAGACAUGGUdTsdT | 304 |
| s | 1206 | ACCUCAGUAUUUUCUAUACdTdT | 219 | ACCUCAGUAUUUUCUAUACdTsdT | 305 |
| as | 1224 | GUAUAGAAAAUACUGAGGUdTdT | 220 | GUAUAGAAAAUACUGAGGUdTsdT | 306 |
| s | 1356 | UGUCUAUCAGUCCAUGACCdTdT | 221 | UGUCUAUCAGUCCAUGACCdTsdT | 307 |
| as | 1374 | GGUCAUGGACUGAUAGACAdTdT | 222 | GGUCAUGGACUGAUAGACAdTsdT | 308 |
| s | 1902 | CAUCCGGACCAAGAUGUUCdTdT | 223 | CAUCCGGACCAAGAUGUUCdTsdT | 309 |
| as | 1920 | GAACAUCUUGGUCCGGAUGdTdT | 224 | GAACAUCUUGGUCCGGAUGdTsdT | 310 |
| s | 1201 | UCUCAACCUCAGUAUUUUCdTdT | 225 | UCUCAACCUCAGUAUUUUCdTsdT | 311 |
| as | 1219 | GAAAAUACUGAGGUUGAGAdTdT | 226 | GAAAAUACUGAGGUUGAGAdTsdT | 312 |
| s | 1901 | UCAUCCGGACCAAGAUGUUdTdT | 227 | UCAUCCGGACCAAGAUGUUdTsdT | 313 |
| as | 1919 | AACAUCUUGGUCCGGAUGAdTdT | 228 | AACAUCUUGGUCCGGAUGAdTsdT | 314 |
| s | 1351 | AAUGGUGUCUAUCAGUCCAdTdT | 229 | AAUGGUGUCUAUCAGUCCAdTsdT | 315 |
| as | 1369 | UGGACUGAUAGACACCAUUdTdT | 230 | UGGACUGAUAGACACCAUUdTsdT | 316 |
| s | 1207 | CCUCAGUAUUUUCUAUACAdTdT | 231 | CCUCAGUAUUUUCUAUACAdTsdT | 317 |
| as | 1225 | UGUAUAGAAAAUACUGAGGdTdT | 232 | UGUAUAGAAAAUACUGAGGdTsdT | 318 |
| s | 2273 | ACUUUGGUCUCUUCUACGCdTdT | 233 | ACUUUGGUCUCUUCUACGCdTsdT | 319 |
| as | 2291 | GCGUAGAAGAGACCAAAGUdTdT | 234 | GCGUAGAAGAGACCAAAGUdTsdT | 320 |
| s | 1198 | CCCUCUCAACCUCAGUAUUdTdT | 235 | CCCUCUCAACCUCAGUAUUdTsdT | 321 |
| as | 1216 | AAUACUGAGGUUGAGAGGGdTdT | 236 | AAUACUGAGGUUGAGAGGGdTsdT | 322 |
| s | 2267 | CCAAACACUUUGGUCUCUUdTdT | 237 | CCAAACACUUUGGUCUCUUdTsdT | 323 |
| as | 2285 | AAGAGACCAAAGUGUUUGGdTdT | 238 | AAGAGACCAAAGUGUUUGGdTsdT | 324 |
| s | 1205 | AACCUCAGUAUUUUCUAUAdTdT | 239 | AACCUCAGUAUUUUCUAUAdTsdT | 325 |
| as | 1223 | UAUAGAAAAUACUGAGGUUdTdT | 240 | UAUAGAAAAUACUGAGGUUdTsdT | 326 |
| s | 1357 | GUCUAUCAGUCCAUGACCAdTdT | 241 | GUCUAUCAGUCCAUGACCAdTsdT | 327 |
| as | 1375 | UGGUCAUGGACUGAUAGACdTdT | 242 | UGGUCAUGGACUGAUAGACdTsdT | 328 |
| s | 824 | UCAGCACCGAGAACAUCUAdTdT | 243 | UCAGCACCGAGAACAUCUAdTsdT | 329 |
| as | 842 | UAGAUGUUCUCGGUGCUGAdTdT | 244 | UAGAUGUUCUCGGUGCUGAdTsdT | 330 |
| s | 1354 | GGUGUCUAUCAGUCCAUGAdTdT | 245 | GGUGUCUAUCAGUCCAUGAdTsdT | 331 |
| as | 1372 | UCAUGGACUGAUAGACACCdTdT | 246 | UCAUGGACUGAUAGACACCdTsdT | 332 |
| s | 1197 | CCCCUCUCAACCUCAGUAUdTdT | 247 | CCCCUCUCAACCUCAGUAUdTsdT | 333 |
| as | 1215 | AUACUGAGGUUGAGAGGGGdTdT | 248 | AUACUGAGGUUGAGAGGGGdTsdT | 334 |
| s | 826 | AGCACCGAGAACAUCUACUdTdT | 249 | AGCACCGAGAACAUCUACUdTsdT | 335 |
| as | 844 | AGUAGAUGUUCUCGGUGCUdTdT | 250 | AGUAGAUGUUCUCGGUGCUdTsdT | 336 |
| s | 825 | CAGCACCGAGAACAUCUACdTdT | 251 | CAGCACCGAGAACAUCUACdTsdT | 337 |
| as | 843 | GUAGAUGUUCUCGGUGCUGdTdT | 252 | GUAGAUGUUCUCGGUGCUGdTsdT | 338 |
| s | 1352 | AUGGUGUCUAUCAGUCCAUdTdT | 253 | AUGGUGUCUAUCAGUCCAUdTsdT | 339 |
| as | 1370 | AUGGACUGAUAGACACCAUdTdT | 254 | AUGGACUGAUAGACACCAUdTsdT | 340 |
| s | 2270 | AACACUUUGGUCUCUUCUAdTdT | 255 | AACACUUUGGUCUCUUCUAdTsdT | 341 |
| as | 2288 | UAGAAGAGACCAAAGUGUUdTdT | 256 | UAGAAGAGACCAAAGUGUUdTsdT | 342 |
| s | 3112 | AGAGACCAGAUCCCUGUCUdTdT | 257 | AGAGACCAGAUCCCUGUCUdTsdT | 343 |
| as | 3130 | AGACAGGGAUCUGGUCUCUdTdT | 258 | AGACAGGGAUCUGGUCUCUdTsdT | 344 |

TABLE 4A
Chemically modified sense and antisense sequences of human SIDT1 dsRNAs
with 3′ dTdT phosphodiester linkage

| Strand ID | | | | |
|---|---|---|---|---|
| (s = | | | | |
| sense; | | Position of | Sequence (5′ to 3′) | SEQ |
| as = | Oligo | 5′ base on | (3′ dTdT phosphodiester | ID |
| antisense) | ID # | transcript | linkage (3′ PO)) | NO: |
| s | A-31481 | 2336 | AccAuGucuGcccuAAuuAdTdT | 345 |
| as | A-31482 | 2354 | uAAUuAGGGcAGAcAUGGUdTdT | 346 |
| s | A-31483 | 1206 | AccucAGuAuuuucuAuAcdTdT | 347 |
| as | A-31484 | 1224 | GuAuAGAAAAuACUGAGGUdTdT | 348 |
| s | A-31485 | 1200 | cucucAAccucAGuAuuuudTdT | 349 |
| as | A-31486 | 1218 | AAAAuACUGAGGUUGAGAGdTdT | 350 |
| s | A-31487 | 1356 | uGucuAucAGuccAuGAccdTdT | 351 |
| as | A-31488 | 1374 | GGUcAUGGACUGAuAGAcAdTdT | 352 |
| s | A-31489 | 1902 | cAuccGGAccAAGAuGuucdTdT | 353 |
| as | A-31490 | 1920 | GAAcAUCUUGGUCCGGAUGdTdT | 354 |
| s | A-31491 | 1201 | ucucAAccucAGuAuuuucdTdT | 355 |
| as | A-31492 | 1219 | GAAAAuACUGAGGUUGAGAdTdT | 356 |
| s | A-31493 | 1901 | ucAuccGGAccAAGAuGuudTdT | 357 |
| as | A-31494 | 1919 | AAcAUCUUGGUCCGGAUGAdTdT | 358 |
| s | A-31495 | 1351 | AAuGGuGucuAucAGuccAdTdT | 359 |
| as | A-31496 | 1369 | UGGACUGAuAGAcACcAUUdTdT | 360 |
| s | A-31497 | 1207 | ccucAGuAuuuucuAuAcAdTdT | 361 |
| as | A-31498 | 1225 | UGuAuAGAAAAuACUGAGGdTdT | 362 |
| s | A-31499 | 2273 | AcuuuGGucucuucuAcGcdTdT | 363 |
| as | A-31500 | 2291 | GCGuAGAAGAGACcAAAGUdTdT | 364 |
| s | A-31501 | 1198 | cccucucAAccucAGuAuudTdT | 365 |
| as | A-31502 | 1216 | AAuACUGAGGUUGAGAGGGdTdT | 366 |
| s | A-31503 | 2267 | ccAAAcAcuuuGGucucuudTdT | 367 |
| as | A-31504 | 2285 | AAGAGACcAAAGUGUUUGGdTdT | 368 |
| s | A-31505 | 1205 | AAccucAGuAuuuucuAuAdTdT | 369 |
| as | A-31506 | 1223 | uAuAGAAAAuACUGAGGUUdTdT | 370 |
| s | A-31507 | 1357 | GucuAucAGuccAuGAccAdTdT | 371 |
| as | A-31508 | 1375 | UGGUcAUGGACUGAuAGACdTdT | 372 |
| s | A-31509 | 824 | ucAGcAccGAGAAcAucuAdTdT | 373 |
| as | A-31510 | 842 | uAGAUGUUCUCGGUGCUGAdTdT | 374 |
| s | A-31511 | 1354 | GGuGucuAucAGuccAuGAdTdT | 375 |
| as | A-31512 | 1372 | UcAUGGACUGAuAGAcACCdTdT | 376 |
| s | A-31513 | 1197 | ccccucucAAccucAGuAudTdT | 377 |
| as | A-31514 | 1215 | AuACUGAGGUUGAGAGGGGdTdT | 378 |
| s | A-31515 | 826 | AGcAccGAGAAcAucuAcudTdT | 379 |
| as | A-31516 | 844 | AGuAGAUGUUCUCGGUGCUdTdT | 380 |
| s | A-31517 | 825 | cAGcAccGAGAAcAucuAcdTdT | 381 |
| as | A-31518 | 843 | GuAGAUGUUCUCGGUGCUGdTdT | 382 |
| s | A-31519 | 1352 | AuGGuGucuAucAGuccAudTdT | 383 |
| as | A-31520 | 1370 | AUGGACUGAuAGAcACcAUdTdT | 384 |
| s | A-31521 | 2270 | AAcAcuuuGGucucuucuAdTdT | 385 |
| as | A-31522 | 2288 | uAGAAGAGACcAAAGUGUUdTdT | 386 |
| s | A-31523 | 3112 | AGAGAccAGAucccuGucudTdT | 387 |
| as | A-31524 | 3130 | AGAcAGGGAUCUGGUCUCUdTdT | 388 |
| s | | 2336 | AccAuGucuGcccuAAuuAdTdT | 389 |
| as | | 2354 | uAAUuAGGGcAGAcAUGGUdTdT | 390 |
| s | | 1206 | AccucAGuAuuuucuAuAcdTdT | 391 |
| as | | 1224 | GuAuAGAAAAuACUGAGGUdTdT | 392 |
| s | | 1356 | uGucuAucAGuccAuGAccdTdT | 393 |
| as | | 1374 | GGUcAUGGACUGAuAGAcAdTdT | 394 |
| s | | 1902 | cAuccGGAccAAGAuGuucdTdT | 395 |
| as | | 1920 | GAAcAUCUUGGUCCGGAUGdTdT | 396 |
| s | | 1201 | ucucAAccucAGuAuuuucdTdT | 397 |
| as | | 1219 | GAAAAuACUGAGGUUGAGAdTdT | 398 |
| s | | 1901 | ucAuccGGAccAAGAuGuudTdT | 399 |
| as | | 1919 | AAcAUCUUGGUCCGGAUGAdTdT | 400 |
| s | | 1351 | AAuGGuGucuAucAGuccAdTdT | 401 |
| as | | 1369 | UGGACUGAuAGAcACcAUUdTdT | 402 |
| s | | 1207 | ccucAGuAuuuucuAuAcAdTdT | 403 |
| as | | 1225 | UGuAuAGAAAAuACUGAGGdTdT | 404 |
| s | | 2273 | AcuuuGGucucuucuAcGcdTdT | 405 |
| as | | 2291 | GCGuAGAAGAGACcAAAGUdTdT | 406 |
| s | | 1198 | cccucucAAccucAGuAuudTdT | 407 |
| as | | 1216 | AAuACUGAGGUUGAGAGGGdTdT | 408 |
| s | | 2267 | ccAAAcAcuuuGGucucuudTdT | 409 |
| as | | 2285 | AAGAGACcAAAGUGUUUGGdTdT | 410 |
| s | | 1205 | AAccucAGuAuuuucuAuAdTdT | 411 |
| as | | 1223 | uAuAGAAAAuACUGAGGUUdTdT | 412 |
| s | | 1357 | GucuAucAGuccAuGAccAdTdT | 413 |
| as | | 1375 | UGGUcAUGGACUGAuAGACdTdT | 414 |
| s | | 824 | ucAGcAccGAGAAcAucuAdTdT | 415 |
| as | | 842 | uAGAUGUUCUCGGUGCUGAdTdT | 416 |
| s | | 1354 | GGuGucuAucAGuccAuGAdTdT | 417 |
| as | | 1372 | UcAUGGACUGAuAGAcACCdTdT | 418 |
| s | | 1197 | ccccucucAAccucAGuAudTdT | 419 |
| as | | 1215 | AuACUGAGGUUGAGAGGGGdTdT | 420 |
| s | | 826 | AGcAccGAGAAcAucuAcudTdT | 421 |
| as | | 844 | AGuAGAUGUUCUCGGUGCUdTdT | 422 |
| s | | 825 | cAGcAccGAGAAcAucuAcdTdT | 423 |
| as | | 843 | GuAGAUGUUCUCGGUGCUGdTdT | 424 |
| s | | 1352 | AuGGuGucuAucAGuccAudTdT | 425 |
| as | | 1370 | AUGGACUGAuAGAcACcAUdTdT | 426 |
| s | | 2270 | AAcAcuuuGGucucuucuAdTdT | 427 |
| as | | 2288 | uAGAAGAGACcAAAGUGUUdTdT | 428 |
| s | | 3112 | AGAGAccAGAucccuGucudTdT | 429 |
| as | | 3130 | AGAcAGGGAUCUGGUCUCUdTdT | 430 |

TABLE 4B
Chemically modified sense and antisense sequences of human SIDT1 dsRNAs
with 3′ dTsdT phosphodiester linkage

| Strand ID | | | | |
|---|---|---|---|---|
| (s = | | Position | Sequence (5′ to 3′) | |
| sense; | | of 5′ base | (3′ dTsdT | SEQ |
| as = | Oligo | on | phosphorothioate linkage | ID |
| antisense) | ID # | transcript | (3′ PS)) | NO: |
| s | A-31027 | 2336 | AccAuGucuGcccuAAuuAdTsdT | 431 |
| as | A-31028 | 2354 | uAAUuAGGGcAGAcAUGGUdTsdT | 432 |
| s | A-31029 | 1206 | AccucAGuAuuuucuAuAcdTsdT | 433 |
| as | A-31030 | 1224 | GuAuAGAAAAuACUGAGGUdTsdT | 434 |
| s | A-31031 | 1200 | cucucAAccucAGuAuuuudTsdT | 435 |
| as | A-31032 | 1218 | AAAAuACUGAGGUUGAGAGdTsdT | 436 |
| s | A-31033 | 1356 | uGucuAucAGuccAuGAccdTsdT | 437 |
| as | A-31034 | 1374 | GGUcAUGGACUGAuAGAcAdTsdT | 438 |
| s | A-31035 | 1902 | cAuccGGAccAAGAuGuucdTsdT | 439 |
| as | A-31036 | 1920 | GAAcAUCUUGGUCCGGAUGdTsdT | 440 |
| s | A-31037 | 1201 | ucucAAccucAGuAuuuucdTsdT | 441 |
| as | A-31038 | 1219 | GAAAAuACUGAGGUUGAGAdTsdT | 442 |
| s | A-31039 | 1901 | ucAuccGGAccAAGAuGuudTsdT | 443 |
| as | A-31040 | 1919 | AAcAUCUUGGUCCGGAUGAdTsdT | 444 |
| s | A-31041 | 1351 | AAuGGuGucuAucAGuccAdTsdT | 445 |
| as | A-31042 | 1369 | UGGACUGAuAGAcACcAUUdTsdT | 446 |
| s | A-31043 | 1207 | ccucAGuAuuuucuAuAcAdTsdT | 447 |
| as | A-31044 | 1225 | UGuAuAGAAAAuACUGAGGdTsdT | 448 |
| s | A-31045 | 2273 | AcuuuGGucucuucuAcGcdTsdT | 449 |
| as | A-31046 | 2291 | GCGuAGAAGAGACcAAAGUdTsdT | 450 |
| s | A-31047 | 1198 | cccucucAAccucAGuAuudTsdT | 451 |
| as | A-31048 | 1216 | AAuACUGAGGUUGAGAGGGdTsdT | 452 |
| s | A-31049 | 2267 | ccAAAcAcuuuGGucucuudTsdT | 453 |
| as | A-31050 | 2285 | AAGAGACcAAAGUGUUUGGdTsdT | 454 |
| s | A-31051 | 1205 | AAccucAGuAuuuucuAuAdTsdT | 455 |
| as | A-31052 | 1223 | uAuAGAAAAuACUGAGGUUdTsdT | 456 |
| s | A-31053 | 1357 | GucuAucAGuccAuGAccAdTsdT | 457 |
| as | A-31054 | 1375 | UGGUcAUGGACUGAuAGACdTsdT | 458 |
| s | A-31055 | 824 | ucAGcAccGAGAAcAucuAdTsdT | 459 |
| as | A-31056 | 842 | uAGAUGUUCUCGGUGCUGAdTsdT | 460 |
| s | A-31057 | 1354 | GGuGucuAucAGuccAuGAdTsdT | 461 |
| as | A-31058 | 1372 | UcAUGGACUGAuAGAcACCdTsdT | 462 |
| s | A-31059 | 1197 | ccccucucAAccucAGuAudTsdT | 463 |
| as | A-31060 | 1215 | AuACUGAGGUUGAGAGGGGdTsdT | 464 |
| s | A-31061 | 826 | AGcAccGAGAAcAucuAcudTsdT | 465 |
| as | A-31062 | 844 | AGuAGAUGUUCUCGGUGCUdTsdT | 466 |
| s | A-31063 | 825 | cAGcAccGAGAAcAucuAcdTsdT | 467 |
| as | A-31064 | 843 | GuAGAUGUUCUCGGUGCUGdTsdT | 468 |
| s | A-31065 | 1352 | AuGGuGucuAucAGuccAudTsdT | 469 |
| as | A-31066 | 1370 | AUGGACUGAuAGAcACcAUdTsdT | 470 |
| s | A-31067 | 2270 | AAcAcuuuGGucucuucuAdTsdT | 471 |
| as | A-31068 | 2288 | uAGAAGAGACcAAAGUGUUdTsdT | 472 |
| s | A-31069 | 3112 | AGAGAccAGAucccuGucudTsdT | 473 |
| as | A-31070 | 3130 | AGAcAGGGAUCUGGUCUCUdTsdT | 474 |

Synthesis of SID-1 Sequences

SID1 sequences were synthesized on MerMade 192 synthesizer at 1 μmol scale.

The sequences were made in two sets. The difference in the two sets was the linkage in the 3′ dTdT overhang. In the first set, a normal phosphodiester linkage was used and for the second set, a phosphorothioate linkage was incorporated in the two base overhang (dTsdT). Chemical modifications were incorporated in the sequence as described below:
All pyrimidines (cytosine and uridine) in the sense strand were replaced with corresponding 2′-O-Methyl bases (2′ O-Methyl C and 2′-O-Methyl U)
In the antisense strand, pyrimidines adjacent to (towards 5′ position) ribo A nucleoside were replaced with their corresponding 2-O-Methyl nucleosides
A two base dTdT extension at the 3′ end of both sense and anti sense sequences was introduced
The sequence file was converted to a text file to make it compatible for loading in the MerMade 192 synthesis software

The synthesis of SID1 sequences used dT solid support and used phosphoramidite chemistry.

The synthesis of the above sequences was performed at 1 μm scale in 96 well plates. The amidite solutions were prepared at 0.1 M concentration and ethyl thio tetrazole (0.6 M in Acetonitrile) was used as activator.

The synthesized sequences were cleaved and deprotected in 96 well plates, using methylamine in the first step and triethylamine.3HF in the second step. The crude sequences thus obtained were precipitated using an acetone: ethanol mix and the pellets were resuspended in 0.02 M sodium acetate buffer. Samples from each sequence were analyzed by LC-MS and the resulting mass data was used to confirm the identity of the sequences. A selected set of samples were also analyzed by IEX chromatography.

The next step in the process was purification. All sequences were purified on an AKTA explorer purification system using a Source 15Q column. A single peak corresponding to the full length sequence was collected in the eluent and was subsequently analyzed for purity by ion exchange chromatography.

The purified sequences were desalted on a Sephadex G25 column using an AKTA purifier. The desalted SID1 sequences were analyzed for concentration and purity. The single strands were then submitted for annealing.

In Vitro Screening

Cell Culture and Transfections:

PC3 human prostate cancer epithelial cells (ATCC; CRL1435™) were grown to near confluence at 37° C. in an atmosphere of 5% CO₂ in Dulbecco's modified Eagle's medium (ATCC) supplemented with 10% FBS, streptomycin, and glutamine (ATCC). Cells were then trypsinized and washed in media with FBS. Reverse transfections were carried out in 96-well plates by adding 5 μl of Opti-MEM to 5 μl of siRNA duplexes per well along with 10 μl of Opti-MEM plus 0.2 μl of Lipofectamine RNAiMax (Invitrogen, Carlsbad Calif. cat #13778-150). 20 minutes after incubating the plates at room temperature, 80 μl of complete growth media without antibiotics containing 2×10⁴ cells were added into each well. Cells were incubated for 24 hours prior to total RNA isolation. Single dose experiments were performed at 5 nM and 10 nM final duplex concentration and dose response experiments were done with 5 nM, 2.5 nM, 1.25 nM, 0.625 nM, 0.3125 nM, 0.15625 nM, 0.078125 nM, 0.039063 nM, 0.019531 nM, and 0.009766 nM siRNA duplexes; and 5 nM, 1 nM, 0.2 nM, 0.04 nM, 0.008 nM, 0.0016 nM, 0.00032 nM, 0.000064 nM, 1.28E-5 nM, 2.56E-6 nM, 5.12E-7 nM, and 1.02E-7 nM siRNA duplexes.

Total RNA Isolation Using RNAqueous0-96 Well Plate Procedure (Applied Biosystem, Forer City Calif., Part #: 1812):

Cells were lysed for 5 minutes in 200 μl of Lysis/Binding Solution. 100 μl of 100% ethanol was added into each cell lysate and the total 300 μl lysates were transferred into one well of “filter plate.” Filter plates were centrifuged at RCF of 10,000-15,000 g for 2 minutes. 300 μl Wash Solution was then added into each well and the plate was centrifuged at RCF of 10,000-15,000 g for 2 minutes. For DNase treatment, 20 μl of DNase mixture was added on top of each filter and the plate was incubated for 15 minutes at room temperature. RNA rebinding was performed by washing filters with 200 μL of Rebinding Mix, and 1 minute later, samples were centrifuged at RCF of 10,000-15,000 g for 2 minutes. Filters were then washed twice with 200 μl of Wash Solution and centrifuged at RCF of 10,000-15,000 g for 2 minutes. A third centrifugation of 2 minutes was then applied after the reservoir unit was emptied and elution of the RNA was performed in a clean culture plate by adding into the filters 50 μL of preheated (80° C.) Nuclease-free Water.

cDNA Synthesis Using ABI High Capacity cDNA Reverse Transcription Kit (Applied Biosystems, Foster City, Calif., Cat #4368813):

A master mix of 2 μl 10× Buffer, 0.8 μl 25× dNTPs, 2 μL Random primers, 1 μl Reverse Transcriptase, 1 μl RNase inhibitor and 3.2 μl of H₂O per reaction were added into 10 μl total RNA (20 ng-2 μg). cDNA was generated using a Bio-Rad C-1000 or S-1000 thermal cycler (Hercules, Calif.) through the following steps: 25° C. 10 min, 37° C. 120 min, 85° C. 5 sec, 4° C. hold.

Real time PCR:

2 μl of cDNA was added to a master mix of 1 μl 18S TaqMan Probe (Eukaryotic 18S rRNA VIC/MGB primer limited Applied Biosystems Cat # 4319413E), 1 μl Sid-1 TaqMan probe (Applied Biosystems cat #Hs00944915 ml) and 10 μl TaqMan Universal PCR Master Mix (Applied Biosystems Cat #4324018) per well in a MicroAmp Optical 96 well plate (Applied Biosystems cat #4326659). Real time PCR was performed in an ABI 7000 Prism or an ABI 7900HT Real Time PCR system (Applied Biosystems) using the ΔΔCt(RQ) assay. All reactions were performed in triplicate.

Real time data were analyzed using the ΔΔCt method and normalized to assays performed from cells transfected with 10 nM BlockIT fluorescent Oligo (Invitrogen Cat # 2013) or 10 nM AD-1955 (a duplex that targets luciferase) to calculate fold change.

Results:

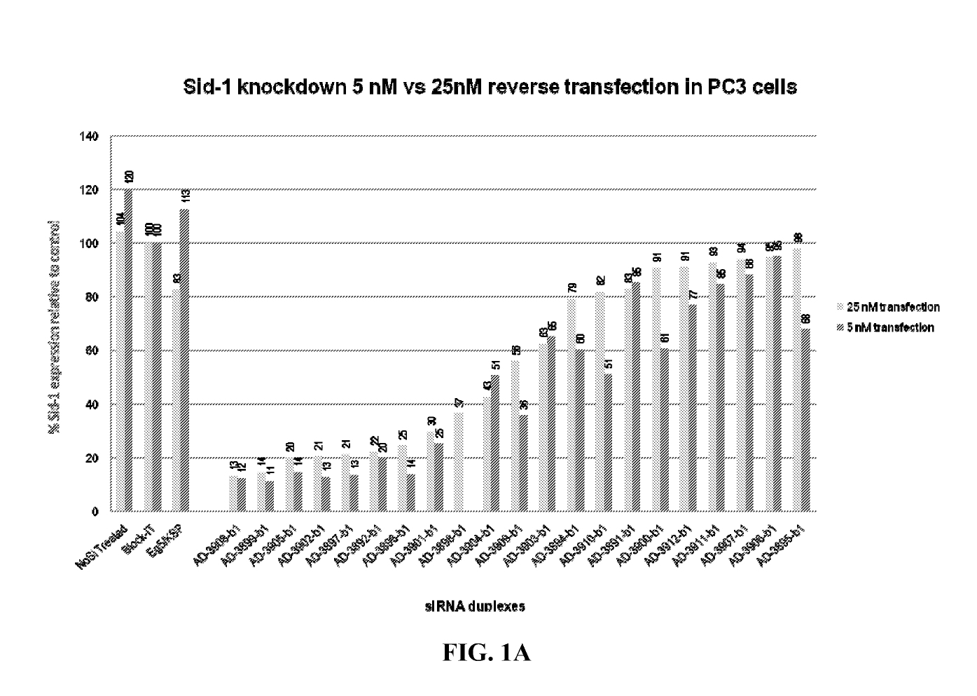

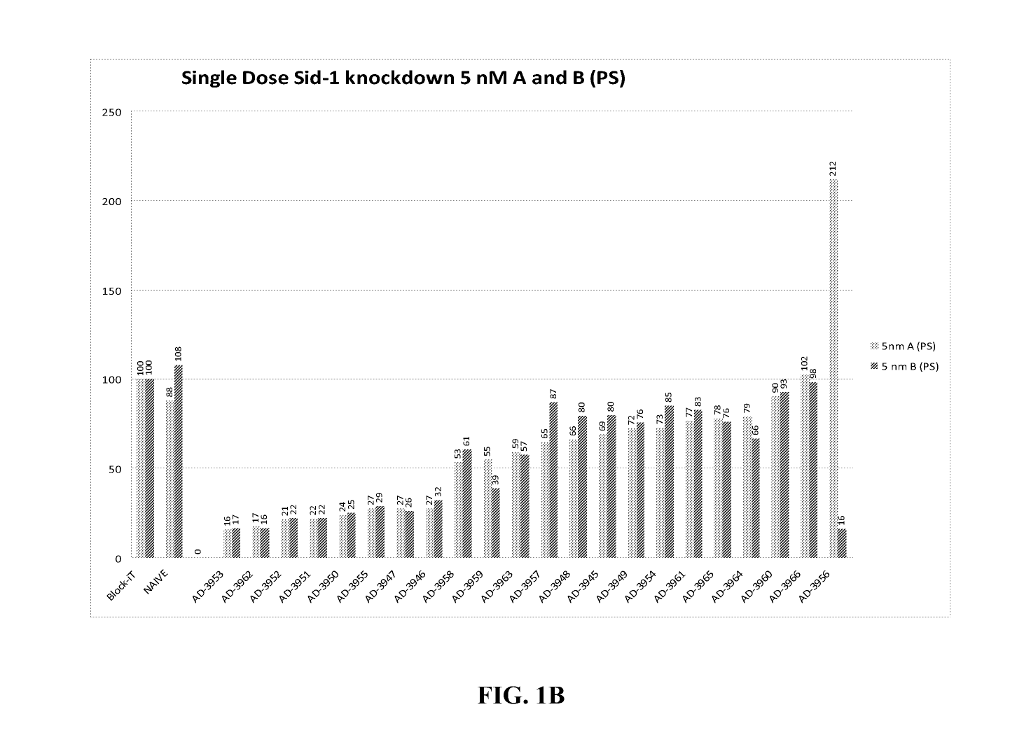

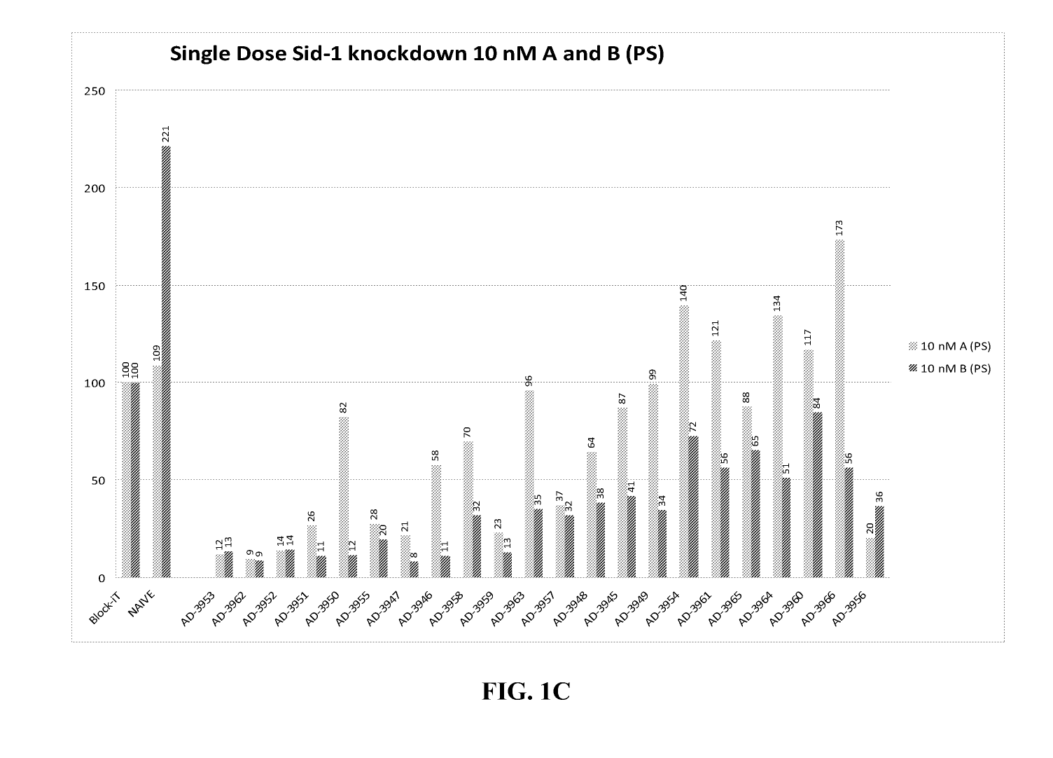

Summary of single dose results for SID-1 duplexes are presented below in Tables 5A and 5B. Single Dose results are expressed as a % of remaining RNA in PC3 cells treated with 5 nM or 25 nM of the tested siRNA duplexes. The same above results are also shown in FIG. 1 as a dose response graph.

TABLE 5A
Single dose results

| SID1 duplexes with 3′ PO | | | Remaining RNA (%) | |
|---|---|---|---|---|
| Duplex ID# | Sense ID# | Antisense ID# | 5 nM | 25 nM |
| AD-3891 | 31481 | 31482 | 85 | 83 |
| AD-3892 | 31483 | 31484 | 20 | 22 |
| AD-3894 | 31487 | 31488 | 60 | 79 |
| AD-3895 | 31489 | 31490 | 68 | 98 |
| AD-3896 | 31491 | 31492 | 14 | 25 |
| AD-3897 | 31493 | 31494 | 13 | 21 |
| AD-3898 | 3195 | 31496 | — | 37 |
| AD-3899 | 31497 | 31498 | 11 | 14 |
| AD-3900 | 31499 | 31500 | 61 | 91 |
| AD-3901 | 31501 | 31502 | 25 | 30 |
| AD-3902 | 31503 | 31504 | 13 | 21 |
| AD-3903 | 31505 | 31506 | 65 | 63 |
| AD-3904 | 31507 | 31508 | 51 | 43 |
| AD-3905 | 31509 | 31510 | 14 | 20 |
| AD-3906 | 31511 | 31512 | 95 | 95 |
| AD-3907 | 31513 | 31514 | 88 | 94 |
| AD-3908 | 31515 | 31516 | 12 | 13 |
| AD-3909 | 31517 | 31518 | 36 | 56 |
| AD-3910 | 31519 | 31520 | 51 | 82 |
| AD-3911 | 31521 | 31522 | 85 | 93 |
| AD-3912 | 31523 | 31524 | 77 | 91 |

TABLE 5B
Single dose results

| SID1 duplexes with 3′ PS | | | Remaining RNA (%) | |
|---|---|---|---|---|
| Duplex ID# | Sense ID# | Antisense ID# | 5 nM | 25 nM |
| AD-3945 | 31027 | 31028 | 74 | 64 |
| AD-3946 | 31029 | 31030 | 29 | 34 |
| AD-3947 | 31031 | 31032 | 27 | 14 |
| AD-3948 | 31033 | 31034 | 73 | 51 |
| AD-3949 | 31035 | 31036 | 74 | 66 |
| AD-3950 | 31037 | 31038 | 25 | 47 |
| AD-3951 | 31039 | 31040 | 22 | 18 |
| AD-3952 | 31041 | 31042 | 22 | 14 |
| AD-3953 | 31043 | 31044 | 17 | 12 |
| AD-3954 | 31045 | 31046 | 79 | 106 |
| AD-3955 | 31047 | 31048 | 28 | 24 |
| AD-3956 | 31049 | 31050 | 16 | 28 |
| AD-3957 | 31051 | 31052 | 76 | 34 |
| AD-3958 | 31053 | 31054 | 57 | 51 |
| AD-3959 | 31055 | 31056 | 47 | 18 |
| AD-3960 | 31057 | 31058 | 91 | 100 |
| AD-3961 | 31059 | 31060 | 80 | 88 |
| AD-3962 | 31061 | 31062 | 17 | 9 |
| AD-3963 | 31063 | 31064 | 58 | 65 |
| AD-3964 | 31065 | 31066 | 72 | 92 |
| AD-3965 | 31067 | 31068 | 77 | 76 |
| AD-3966 | 31069 | 31070 | 100 | 114 |

The 10 3′ PS Sid-1 duplexes demonstrating the best IC50 results are presented below in Tables 6A and 6B. The four duplexes that were identified as having the best IC50's (AD-3947, AD-3946, AD-3962, and AD-3953) were tested for immunostimulatory effects (see below).

TABLE 6A
Optimal IC50 results for 3′ PS SID-1 duplexes

| | Rating: | | | | |
|---|---|---|---|---|---|
| | 3 | 4 | 2 | 5 | 1 |
| siRNA [nM] | AD-3962 | AD-3953 | AD-3946 | AD-3956 | AD-3947 |
| 5 | 11 | 10 | 19 | 12 | 21 |
| 1 | 13 | 24 | 22 | 17 | 19 |
| 0.2 | 21 | 23 | 33 | 21 | 24 |
| 0.04 | 44 | 37 | 53 | 42 | 28 |
| 0.008 | 51 | 45 | 60 | 67 | 45 |
| 0.0016 | 69 | 60 | 62 | 103 | 56 |
| 0.00032 | 65 | 82 | 56 | 77 | 59 |
| 0.000064 | 70 | 102 | 57 | 185 | 85 |
| 0.0000128 | 77 | 72 | 58 | 137 | 74 |
| 0.00000256 | 82 | 77 | 67 | 143 | 94 |
| 5.12E−07 | 66 | 108 | 72 | 87 | 86 |
| 1.02E−07 | 133 | 115 | 104 | 110 | 121 |
| Duplex | AD-3962 | AD-3953 | AD-3946 | AD-3956 | AD-3947 |
| IC50 [nM]: | 0.002631 | 0.006406 | 0.002344 | 0.020133 | 0.001545 |

TABLE 6B
Optimal IC50 results for 3′ PS SID-1 duplexes

| | Rating | | | | |
|---|---|---|---|---|---|
| | 7 | 8 | 10 | 9 | 6 |
| siRNA [nM] | AD-3950 | AD-3951 | AD-3952 | AD-3955 | AD-3959 |
| 5 | 17 | 20 | 25 | 35 | 16 |
| 2.5 | 17 | 16 | 65 | 36 | 18 |
| 1.25 | 22 | 20 | 30 | 45 | 34 |
| 0.625 | 25 | 24 | 56 | 35 | 24 |
| 0.3125 | 31 | 48 | 152 | 47 | 31 |
| 0.15625 | 92 | 78 | 271 | 111 | 49 |
| 0.078125 | 40 | 57 | 126 | 184 | 49 |
| 0.0390625 | 71 | 134 | 319 | 111 | 52 |
| 0.01953125 | 53 | 73 | 213 | 175 | 54 |
| 0.009765625 | 73 | 81 | 329 | 114 | 56 |
| Duplex | AD-3950 | AD-3951 | AD-3952 | AD-3955 | AD-3959 |
| IC50 [nM]: | 0.0670 | 0.2883 | 0.7455 | 0.7414 | 0.0662 |

Immunostimulatory Assays:

Screening SID-1 siRNA Sequences for Immunostimulatory Ability

Human peripheral blood mononuclear cells (PBMC) were isolated from freshly collected buffy coats obtained from healthy donors (Research Blood Components, Inc., Boston, Mass.) by a standard Ficoll-Hypaque density centrifugation. Freshly isolated cells (1×10⁵/well) were seeded in 96-well plates and cultured in RPMI 1640 GlutaMax medium (Invitrogen) supplemented with 10% heat-inactivated fetal bovine serum and 1% antibiotic/antimycotic (Invitrogen).

siRNAs were transfected into PBMC using two separate transfection reagents: GenePorter 2 (GP2; Genlantis) and DOTAP (Roche Applied Science). The transfection reagent was first diluted in Opti-MEM (Invitrogen) for 5 minutes before mixing with an equal volume of Opti-MEM containing the siRNA. siRNA/transfection reagent complexes were incubated as specified by the reagent manufacturers' instructions and subsequently added to PBMC. siRNAs were used at a final concentration of 133 nM.

IFN-α and TNF-α cytokines were detected and quantified in GP2- and DOTAP-transfected culture supernatants, respectively, with commercially available ELISA kits from Bender MedSystems (Vienna, Austria).

Results:

Summary of SID-1 siRNA IFN-α/TNF-α cytokine stimulation results are presented below in Table 7. IFN-α/TNF-α cytokine stimulation results are presented in % to AD-5048 (positive control). Values shown in Table 7 are expressed as the range of values obtained with two separate PBMC donors.

TABLE 7
Summary of SID-1 siRNA IFN-α/TNF-α
cytokine stimulation results

| ID# | % IFN-alpha/AD-5048 | % TNF-alpha/AD-5048 |
|---|---|---|
| AD-3946 | 6-23 | 0 |
| AD-3947 | 6-17 | 0 |
| AD-3953 | 4-14 | 0 |
| AD-3962 | 20-36 | 0 |

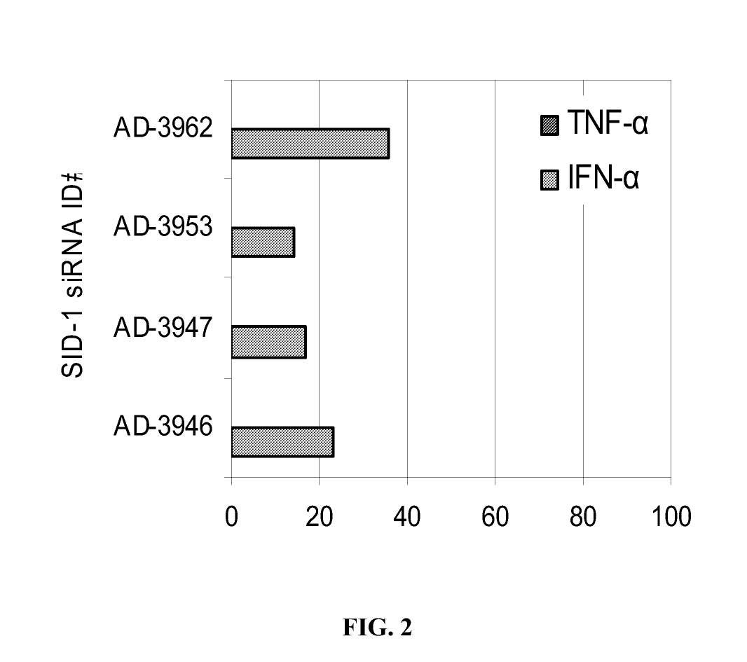

The same above results are also shown in FIG. 2 as a graph. IFN-α/TNF-α cytokine stimulation results are presented in % of cytokine production relative to AD-5048 (positive control). Values shown in FIG. 2 represent the maximum percent cytokine production detected from screening on two individual PBMC donors.

### Example 3

### Inhibition of SID-1 or SID-1 Homologs In Vivo

A non-human animal subject (e.g., a mouse, rat, guinea pig, or monkey) or a human subject is treated with a pharmaceutical composition comprising a pharmaceutical formulation of an siRNA targeting a SID-1 gene.

At time zero, a suitable first dose of the composition is administered to the subject. The composition is formulated as described herein. After a period of time, the subject's condition is evaluated, e.g., by decrease in symptoms and the like. This measurement can be accompanied by a measurement of SID-1 gene expression in said subject, and/or the products of the successful siRNA-targeting of SID-1 gene expression. Other relevant criteria can also be measured, such as the increased or decreased uptake of a particular agent by a cell in the subject. The number and strength of doses are adjusted according to the subject's needs.

After treatment, the subject's condition is compared to the condition existing prior to the treatment, or relative to the condition of a similarly afflicted but untreated subject.

Other embodiments are in the claims.

1. A double-stranded ribonucleic acid (dsRNA), wherein said dsRNA comprises a sense strand and an antisense strand, each less than 30 nucleotides in length, wherein said sense strand comprises at least 15 contiguous nucleotides from SEQ ID NO: 5 and wherein said antisense strand comprises at least 15 contiguous nucleotides from SEQ ID NO: 6, and wherein said antisense strand is complementary to at least 15 nucleotides of said sense strand and complementary to at least a part of a mRNA encoding Systemic RNA Interference Defective-1 (SID-1), and wherein said region of complementarity between said antisense strand and said mRNA is between 15 and 30 contiguous nucleotides in length.

2. The dsRNA of claim 1, wherein at least one nucleotide of said sense strand or said antisense strand is a modified nucleotide.

3. The dsRNA of claim 2, wherein said modified nucleotides is chosen from the group consisting of: a 2′-O-methyl modified nucleotide, a nucleotide comprising a 5′-phosphorothioate group, and a terminal nucleotide linked to a cholesteryl derivative or dodecanoic acid bisdecylamide group.

4. The dsRNA of claim 2, wherein said modified nucleotide is chosen from the group consisting of: a 2′-deoxy-2′-fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide, a phosphoramidate, and a non-natural base comprising nucleotide.

5. The dsRNA of claim 1, comprising a phosphorothioate or a 2′-modified nucleotide.

6. The dsRNA of claim 1, wherein the region of complementary between said antisense strand and said mRNA is at least 19 nucleotides in length.

7. The dsRNA of claim 1, wherein the region of complementarity is between 19 and 21 nucleotides in length.

8. The dsRNA of claim 1, comprising a nucleotide overhang having 1 to 4 nucleotides.

9. The dsRNA of claim 8, wherein the nucleotide overhang is at the 3′-end of the antisense strand of the dsRNA.

10. (canceled)

11. The dsRNA of claim 1, wherein the dsRNA reduces the amount of SID-1 mRNA present in cultured human cells.

12. The dsRNA of claim 1, wherein the dsRNA reduces the amount of SID-1 mRNA in cultured PC3 cells after incubation with the dsRNA by more than 60% compared to cells that have not been incubated with the dsRNA.

13. A vector comprising a nucleotide sequence that encodes at least one strand of a dsRNA of claim 1.

14. A cell comprising the vector of claim 13.

15. A pharmaceutical composition for inhibiting the expression of SID-1, comprising a dsRNA of claim 1 and a pharmaceutically acceptable carrier.

16. The pharmaceutical composition of claim 15, wherein said sense strand of said dsRNA consists of the sequence of SEQ ID NO: 435 and said antisense strand of said dsRNA consists of the sequence of SEQ ID NO: 436.

17. A pharmaceutical composition for inhibiting SID-1 expression, comprising a dsRNA of claim 15 and a lipid formulation.

18. A pharmaceutical composition of claim 17, wherein said sense strand of said dsRNA consists of the sequence of SEQ ID NO: 435 and said antisense strand of said dsRNA consists of the sequence of SEQ ID NO: 436.

19. A method of reducing the amount of SID-1 RNA in a cell comprising:
(a) contacting the cell with an anti-SID-1 dsRNA wherein said anti-SID1 dsRNA is a dsRNA of claim 1, and
(b) maintaining the contact established in step (a) for a time sufficient to mediate cleavage of SID-1 RNA in the cell,
thereby reducing the amount of SID-1 RNA in the cell.

20. The method of claim 19, wherein the method is performed in vitro.

21. The method of claim 19, wherein step (a) comprises introducing the dsRNA into the cell by lipofection.

22. A method of inhibiting RNA interference (RNAi) in a cell comprising:
(a) contacting the cell with an anti-SID-1 dsRNA wherein said anti-SID-1 dsRNA is a dsRNA of claim 1, and
(b) maintaining the contact established in step (a) for a time sufficient to inhibit uptake of a second dsRNA into the cell,
thereby inhibiting RNAi in the cell.

23. The method of claim 19, wherein TNF-α expression is not detectably increased following administration of said anti-SID-1 dsRNA.

24. The method of claim 19, wherein IFN-α expression is increased less than 20% following administration of said anti-SID-1 dsRNA, as compared to a control dsRNA.

25. The method of claim 19, wherein the IC50 of said anti-SID-1 dsRNA is less than 0.01 nM.

26. The method of claim 25, wherein the IC50 of said anti-SID-1 dsRNA is less than 0.004 nM.

27. The method of claim 26, wherein the IC50 of said anti-SID-1 dsRNA is between 0.001 and 0.002 nM.


SEQUENCE LISTING (474 sequences)

SEQ ID NO: 1 - RNA, 19 nt, Artificial Sequence
  Description of Artificial Sequence Synthetic oligonucleotide
accaugucugcccuaauua

SEQ ID NO: 2 - RNA, 19 nt, Artificial Sequence
  Description of Artificial Sequence Synthetic oligonucleotide
uaauuagggcagacauggu

SEQ ID NO: 3 - RNA, 19 nt, Artificial Sequence
  Description of Artificial Sequence Synthetic oligonucleotide
accucaguauuuucuauac

SEQ ID NO: 4 - RNA, 19 nt, Artificial Sequence
  Description of Artificial Sequence Synthetic oligonucleotide
guauagaaaauacugaggu

SEQ ID NO: 5 - RNA, 19 nt, Artificial Sequence
  Description of Artificial Sequence Synthetic oligonucleotide
cucucaaccucaguauuuu

SEQ ID NO: 6 - RNA, 19 nt, Artificial Sequence
  Description of Artificial Sequence Synthetic oligonucleotide
aaaauacugagguugagag

SEQ ID NO: 7 - RNA, 19 nt, Artificial Sequence
  Description of Artificial Sequence Synthetic oligonucleotide
ugucuaucaguccaugacc

SEQ ID NO: 8 - RNA, 19 nt, Artificial Sequence
  Description of Artificial Sequence Synthetic oligonucleotide
ggucauggacugauagaca

SEQ ID NO: 9 - RNA, 19 nt, Artificial Sequence
  Description of Artificial Sequence Synthetic oligonucleotide
cauccggaccaagauguuc

SEQ ID NO: 10 - RNA, 19 nt, Artificial Sequence
  Description of Artificial Sequence Synthetic oligonucleotide
gaacaucuugguccggaug

SEQ ID NO: 11 - RNA, 19 nt, Artificial Sequence
  Description of Artificial Sequence Synthetic oligonucleotide
ucucaaccucaguauuuuc

SEQ ID NO: 12 - RNA, 19 nt, Artificial Sequence
  Description of Artificial Sequence Synthetic oligonucleotide
gaaaauacugagguugaga

SEQ ID NO: 13 - RNA, 19 nt, Artificial Sequence
  Description of Artificial Sequence Synthetic oligonucleotide
ucauccggaccaagauguu

SEQ ID NO: 14 - RNA, 19 nt, Artificial Sequence
  Description of Artificial Sequence Synthetic oligonucleotide
aacaucuugguccggauga

SEQ ID NO: 15 - RNA, 19 nt, Artificial Sequence
  Description of Artificial Sequence Synthetic oligonucleotide
aauggugucuaucagucca

SEQ ID NO: 16 - RNA, 19 nt, Artificial Sequence
  Description of Artificial Sequence Synthetic oligonucleotide
uggacugauagacaccauu

SEQ ID NO: 17 - RNA, 19 nt, Artificial Sequence
  Description of Artificial Sequence Synthetic oligonucleotide
ccucaguauuuucuauaca

SEQ ID NO: 18 - RNA, 19 nt, Artificial Sequence
  Description of Artificial Sequence Synthetic oligonucleotide
uguauagaaaauacugagg

SEQ ID NO: 19 - RNA, 19 nt, Artificial Sequence
  Description of Artificial Sequence Synthetic oligonucleotide
acuuuggucucuucuacgc

SEQ ID NO: 20 - RNA, 19 nt, Artificial Sequence
  Description of Artificial Sequence Synthetic oligonucleotide
gcguagaagagaccaaagu

SEQ ID NO: 21 - RNA, 19 nt, Artificial Sequence
  Description of Artificial Sequence Synthetic oligonucleotide
cccucucaaccucaguauu

SEQ ID NO: 22 - RNA, 19 nt, Artificial Sequence
  Description of Artificial Sequence Synthetic oligonucleotide
aauacugagguugagaggg

SEQ ID NO: 23 - RNA, 19 nt, Artificial Sequence
  Description of Artificial Sequence Synthetic oligonucleotide
ccaaacacuuuggucucuu

SEQ ID NO: 24 - RNA, 19 nt, Artificial Sequence
  Description of Artificial Sequence Synthetic oligonucleotide
aagagaccaaaguguuugg

SEQ ID NO: 25 - RNA, 19 nt, Artificial Sequence
  Description of Artificial Sequence Synthetic oligonucleotide
aaccucaguauuuucuaua

SEQ ID NO: 26 - RNA, 19 nt, Artificial Sequence
  Description of Artificial Sequence Synthetic oligonucleotide
uauagaaaauacugagguu

SEQ ID NO: 27 - RNA, 19 nt, Artificial Sequence
  Description of Artificial Sequence Synthetic oligonucleotide
gucuaucaguccaugacca

SEQ ID NO: 28 - RNA, 19 nt, Artificial Sequence
  Description of Artificial Sequence Synthetic oligonucleotide
uggucauggacugauagac

SEQ ID NO: 29 - RNA, 19 nt, Artificial Sequence
  Description of Artificial Sequence Synthetic oligonucleotide
ucagcaccgagaacaucua

SEQ ID NO: 30 - RNA, 19 nt, Artificial Sequence
  Description of Artificial Sequence Synthetic oligonucleotide
uagauguucucggugcuga

SEQ ID NO: 31 - RNA, 19 nt, Artificial Sequence
  Description of Artificial Sequence Synthetic oligonucleotide
ggugucuaucaguccauga

SEQ ID NO: 32 - RNA, 19 nt, Artificial Sequence
  Description of Artificial Sequence Synthetic oligonucleotide
ucauggacugauagacacc

SEQ ID NO: 33 - RNA, 19 nt, Artificial Sequence
  Description of Artificial Sequence Synthetic oligonucleotide
ccccucucaaccucaguau

SEQ ID NO: 34 - RNA, 19 nt, Artificial Sequence
  Description of Artificial Sequence Synthetic oligonucleotide
auacugagguugagagggg

SEQ ID NO: 35 - RNA, 19 nt, Artificial Sequence
  Description of Artificial Sequence Synthetic oligonucleotide
agcaccgagaacaucuacu

SEQ ID NO: 36 - RNA, 19 nt, Artificial Sequence
  Description of Artificial Sequence Synthetic oligonucleotide
aguagauguucucggugcu

SEQ ID NO: 37 - RNA, 19 nt, Artificial Sequence
  Description of Artificial Sequence Synthetic oligonucleotide
cagcaccgagaacaucuac

SEQ ID NO: 38 - RNA, 19 nt, Artificial Sequence
  Description of Artificial Sequence Synthetic oligonucleotide
guagauguucucggugcug

SEQ ID NO: 39 - RNA, 19 nt, Artificial Sequence
  Description of Artificial Sequence Synthetic oligonucleotide
auggugucuaucaguccau

SEQ ID NO: 40 - RNA, 19 nt, Artificial Sequence
  Description of Artificial Sequence Synthetic oligonucleotide
auggacugauagacaccau

SEQ ID NO: 41 - RNA, 19 nt, Artificial Sequence
  Description of Artificial Sequence Synthetic oligonucleotide
aacacuuuggucucuucua

SEQ ID NO: 42 - RNA, 19 nt, Artificial Sequence
  Description of Artificial Sequence Synthetic oligonucleotide
uagaagagaccaaaguguu

SEQ ID NO: 43 - RNA, 19 nt, Artificial Sequence
  Description of Artificial Sequence Synthetic oligonucleotide
agagaccagaucccugucu

SEQ ID NO: 44 - RNA, 19 nt, Artificial Sequence
  Description of Artificial Sequence Synthetic oligonucleotide
agacagggaucuggucucu

SEQ ID NO: 45 - RNA, 19 nt, Artificial Sequence
  Description of Artificial Sequence Synthetic oligonucleotide
accaugucugcccuaauua

SEQ ID NO: 46 - RNA, 19 nt, Artificial Sequence
  Description of Artificial Sequence Synthetic oligonucleotide
uaauuagggcagacauggu

SEQ ID NO: 47 - RNA, 19 nt, Artificial Sequence
  Description of Artificial Sequence Synthetic oligonucleotide
accucaguauuuucuauac

SEQ ID NO: 48 - RNA, 19 nt, Artificial Sequence
  Description of Artificial Sequence Synthetic oligonucleotide
guauagaaaauacugaggu

SEQ ID NO: 49 - RNA, 19 nt, Artificial Sequence
  Description of Artificial Sequence Synthetic oligonucleotide
ugucuaucaguccaugacc

SEQ ID NO: 50 - RNA, 19 nt, Artificial Sequence
  Description of Artificial Sequence Synthetic oligonucleotide
ggucauggacugauagaca

SEQ ID NO: 51 - RNA, 19 nt, Artificial Sequence
  Description of Artificial Sequence Synthetic oligonucleotide
cauccggaccaagauguuc

SEQ ID NO: 52 - RNA, 19 nt, Artificial Sequence
  Description of Artificial Sequence Synthetic oligonucleotide
gaacaucuugguccggaug

SEQ ID NO: 53 - RNA, 19 nt, Artificial Sequence
  Description of Artificial Sequence Synthetic oligonucleotide
ucucaaccucaguauuuuc

SEQ ID NO: 54 - RNA, 19 nt, Artificial Sequence
  Description of Artificial Sequence Synthetic oligonucleotide
gaaaauacugagguugaga

SEQ ID NO: 55 - RNA, 19 nt, Artificial Sequence
  Description of Artificial Sequence Synthetic oligonucleotide
ucauccggaccaagauguu

SEQ ID NO: 56 - RNA, 19 nt, Artificial Sequence
  Description of Artificial Sequence Synthetic oligonucleotide
aacaucuugguccggauga

SEQ ID NO: 57 - RNA, 19 nt, Artificial Sequence
  Description of Artificial Sequence Synthetic oligonucleotide
aauggugucuaucagucca

SEQ ID NO: 58 - RNA, 19 nt, Artificial Sequence
  Description of Artificial Sequence Synthetic oligonucleotide
uggacugauagacaccauu

SEQ ID NO: 59 - RNA, 19 nt, Artificial Sequence
  Description of Artificial Sequence Synthetic oligonucleotide
ccucaguauuuucuauaca

SEQ ID NO: 60 - RNA, 19 nt, Artificial Sequence
  Description of Artificial Sequence Synthetic oligonucleotide
uguauagaaaauacugagg

SEQ ID NO: 61 - RNA, 19 nt, Artificial Sequence
  Description of Artificial Sequence Synthetic oligonucleotide
acuuuggucucuucuacgc

SEQ ID NO: 62 - RNA, 19 nt, Artificial Sequence
  Description of Artificial Sequence Synthetic oligonucleotide
gcguagaagagaccaaagu

SEQ ID NO: 63 - RNA, 19 nt, Artificial Sequence
  Description of Artificial Sequence Synthetic oligonucleotide
cccucucaaccucaguauu

SEQ ID NO: 64 - RNA, 19 nt, Artificial Sequence
  Description of Artificial Sequence Synthetic oligonucleotide
aauacugagguugagaggg

SEQ ID NO: 65 - RNA, 19 nt, Artificial Sequence
  Description of Artificial Sequence Synthetic oligonucleotide
ccaaacacuuuggucucuu

SEQ ID NO: 66 - RNA, 19 nt, Artificial Sequence
  Description of Artificial Sequence Synthetic oligonucleotide
aagagaccaaaguguuugg

SEQ ID NO: 67 - RNA, 19 nt, Artificial Sequence
  Description of Artificial Sequence Synthetic oligonucleotide
aaccucaguauuuucuaua

SEQ ID NO: 68 - RNA, 19 nt, Artificial Sequence
  Description of Artificial Sequence Synthetic oligonucleotide
uauagaaaauacugagguu

SEQ ID NO: 69 - RNA, 19 nt, Artificial Sequence
  Description of Artificial Sequence Synthetic oligonucleotide
gucuaucaguccaugacca

SEQ ID NO: 70 - RNA, 19 nt, Artificial Sequence
  Description of Artificial Sequence Synthetic oligonucleotide
uggucauggacugauagac

SEQ ID NO: 71 - RNA, 19 nt, Artificial Sequence
  Description of Artificial Sequence Synthetic oligonucleotide
ucagcaccgagaacaucua

SEQ ID NO: 72 - RNA, 19 nt, Artificial Sequence
  Description of Artificial Sequence Synthetic oligonucleotide
uagauguucucggugcuga

SEQ ID NO: 73 - RNA, 19 nt, Artificial Sequence
  Description of Artificial Sequence Synthetic oligonucleotide
ggugucuaucaguccauga

SEQ ID NO: 74 - RNA, 19 nt, Artificial Sequence
  Description of Artificial Sequence Synthetic oligonucleotide
ucauggacugauagacacc

SEQ ID NO: 75 - RNA, 19 nt, Artificial Sequence
  Description of Artificial Sequence Synthetic oligonucleotide
ccccucucaaccucaguau

SEQ ID NO: 76 - RNA, 19 nt, Artificial Sequence
  Description of Artificial Sequence Synthetic oligonucleotide
auacugagguugagagggg

SEQ ID NO: 77 - RNA, 19 nt, Artificial Sequence
  Description of Artificial Sequence Synthetic oligonucleotide
agcaccgagaacaucuacu

SEQ ID NO: 78 - RNA, 19 nt, Artificial Sequence
  Description of Artificial Sequence Synthetic oligonucleotide
aguagauguucucggugcu

SEQ ID NO: 79 - RNA, 19 nt, Artificial Sequence
  Description of Artificial Sequence Synthetic oligonucleotide
cagcaccgagaacaucuac

SEQ ID NO: 80 - RNA, 19 nt, Artificial Sequence
  Description of Artificial Sequence Synthetic oligonucleotide
guagauguucucggugcug

SEQ ID NO: 81 - RNA, 19 nt, Artificial Sequence
  Description of Artificial Sequence Synthetic oligonucleotide
auggugucuaucaguccau

SEQ ID NO: 82 - RNA, 19 nt, Artificial Sequence
  Description of Artificial Sequence Synthetic oligonucleotide
auggacugauagacaccau

SEQ ID NO: 83 - RNA, 19 nt, Artificial Sequence
  Description of Artificial Sequence Synthetic oligonucleotide
aacacuuuggucucuucua

SEQ ID NO: 84 - RNA, 19 nt, Artificial Sequence
  Description of Artificial Sequence Synthetic oligonucleotide
uagaagagaccaaaguguu

SEQ ID NO: 85 - RNA, 19 nt, Artificial Sequence
  Description of Artificial Sequence Synthetic oligonucleotide
agagaccagaucccugucu

SEQ ID NO: 86 - RNA, 19 nt, Artificial Sequence
  Description of Artificial Sequence Synthetic oligonucleotide
agacagggaucuggucucu

SEQ ID NO: 87 - DNA, 21 bp, Artificial Sequence
  Description of Artificial Sequence Synthetic oligonucleotide
accaugucugcccuaauuann

SEQ ID NO: 88 - DNA, 21 bp, Artificial Sequence
  Description of Artificial Sequence Synthetic oligonucleotide
uaauuagggcagacauggunn

SEQ ID NO: 89 - DNA, 21 bp, Artificial Sequence
  Description of Artificial Sequence Synthetic oligonucleotide
accucaguauuuucuauacnn

SEQ ID NO: 90 - DNA, 21 bp, Artificial Sequence
  Description of Artificial Sequence Synthetic oligonucleotide
guauagaaaauacugaggunn

SEQ ID NO: 91 - DNA, 21 bp, Artificial Sequence
  Description of Artificial Sequence Synthetic oligonucleotide
cucucaaccucaguauuuunn

SEQ ID NO: 92 - DNA, 21 bp, Artificial Sequence
  Description of Artificial Sequence Synthetic oligonucleotide
aaaauacugagguugagagnn

SEQ ID NO: 93 - DNA, 21 bp, Artificial Sequence
  Description of Artificial Sequence Synthetic oligonucleotide
ugucuaucaguccaugaccnn

SEQ ID NO: 94 - DNA, 21 bp, Artificial Sequence
  Description of Artificial Sequence Synthetic oligonucleotide
ggucauggacugauagacann

SEQ ID NO: 95 - DNA, 21 bp, Artificial Sequence
  Description of Artificial Sequence Synthetic oligonucleotide
cauccggaccaagauguucnn

SEQ ID NO: 96 - DNA, 21 bp, Artificial Sequence
  Description of Artificial Sequence Synthetic oligonucleotide
gaacaucuugguccggaugnn

SEQ ID NO: 97 - DNA, 21 bp, Artificial Sequence
  Description of Artificial Sequence Synthetic oligonucleotide
ucucaaccucaguauuuucnn

SEQ ID NO: 98 - DNA, 21 bp, Artificial Sequence
  Description of Artificial Sequence Synthetic oligonucleotide
gaaaauacugagguugagann

SEQ ID NO: 99 - DNA, 21 bp, Artificial Sequence
  Description of Artificial Sequence Synthetic oligonucleotide
ucauccggaccaagauguunn

SEQ ID NO: 100 - DNA, 21 bp, Artificial Sequence
  Description of Artificial Sequence Synthetic oligonucleotide
aacaucuugguccggaugann

SEQ ID NO: 101 - DNA, 21 bp, Artificial Sequence
  Description of Artificial Sequence Synthetic oligonucleotide
aauggugucuaucaguccann

SEQ ID NO: 102 - DNA, 21 bp, Artificial Sequence
  Description of Artificial Sequence Synthetic oligonucleotide
uggacugauagacaccauunn

SEQ ID NO: 103 - DNA, 21 bp, Artificial Sequence
  Description of Artificial Sequence Synthetic oligonucleotide
ccucaguauuuucuauacann

SEQ ID NO: 104 - DNA, 21 bp, Artificial Sequence
  Description of Artificial Sequence Synthetic oligonucleotide
uguauagaaaauacugaggnn

SEQ ID NO: 105 - DNA, 21 bp, Artificial Sequence
  Description of Artificial Sequence Synthetic oligonucleotide
acuuuggucucuucuacgcnn

SEQ ID NO: 106 - DNA, 21 bp, Artificial Sequence
  Description of Artificial Sequence Synthetic oligonucleotide
gcguagaagagaccaaagunn

SEQ ID NO: 107 - DNA, 21 bp, Artificial Sequence
  Description of Artificial Sequence Synthetic oligonucleotide
cccucucaaccucaguauunn

SEQ ID NO: 108 - DNA, 21 bp, Artificial Sequence
  Description of Artificial Sequence Synthetic oligonucleotide
aauacugagguugagagggnn

SEQ ID NO: 109 - DNA, 21 bp, Artificial Sequence
  Description of Artificial Sequence Synthetic oligonucleotide
ccaaacacuuuggucucuunn

SEQ ID NO: 110 - DNA, 21 bp, Artificial Sequence
  Description of Artificial Sequence Synthetic oligonucleotide
aagagaccaaaguguuuggnn

SEQ ID NO: 111 - DNA, 21 bp, Artificial Sequence
  Description of Artificial Sequence Synthetic oligonucleotide
aaccucaguauuuucuauann

SEQ ID NO: 112 - DNA, 21 bp, Artificial Sequence
  Description of Artificial Sequence Synthetic oligonucleotide
uauagaaaauacugagguunn

SEQ ID NO: 113 - DNA, 21 bp, Artificial Sequence
  Description of Artificial Sequence Synthetic oligonucleotide
gucuaucaguccaugaccann

SEQ ID NO: 114 - DNA, 21 bp, Artificial Sequence
  Description of Artificial Sequence Synthetic oligonucleotide
uggucauggacugauagacnn

SEQ ID NO: 115 - DNA, 21 bp, Artificial Sequence
  Description of Artificial Sequence Synthetic oligonucleotide
ucagcaccgagaacaucuann

SEQ ID NO: 116 - DNA, 21 bp, Artificial Sequence
  Description of Artificial Sequence Synthetic oligonucleotide
uagauguucucggugcugann

SEQ ID NO: 117 - DNA, 21 bp, Artificial Sequence
  Description of Artificial Sequence Synthetic oligonucleotide
ggugucuaucaguccaugann

SEQ ID NO: 118 - DNA, 21 bp, Artificial Sequence
  Description of Artificial Sequence Synthetic oligonucleotide
ucauggacugauagacaccnn

SEQ ID NO: 119 - DNA, 21 bp, Artificial Sequence
  Description of Artificial Sequence Synthetic oligonucleotide
ccccucucaaccucaguaunn

SEQ ID NO: 120 - DNA, 21 bp, Artificial Sequence
  Description of Artificial Sequence Synthetic oligonucleotide
auacugagguugagaggggnn

SEQ ID NO: 121 - DNA, 21 bp, Artificial Sequence
  Description of Artificial Sequence Synthetic oligonucleotide
agcaccgagaacaucuacunn

SEQ ID NO: 122 - DNA, 21 bp, Artificial Sequence
  Description of Artificial Sequence Synthetic oligonucleotide
aguagauguucucggugcunn

SEQ ID NO: 123 - DNA, 21 bp, Artificial Sequence
  Description of Artificial Sequence Synthetic oligonucleotide
cagcaccgagaacaucuacnn

SEQ ID NO: 124 - DNA, 21 bp, Artificial Sequence
  Description of Artificial Sequence Synthetic oligonucleotide
guagauguucucggugcugnn

SEQ ID NO: 125 - DNA, 21 bp, Artificial Sequence
  Description of Artificial Sequence Synthetic oligonucleotide
auggugucuaucaguccaunn

SEQ ID NO: 126 - DNA, 21 bp, Artificial Sequence
  Description of Artificial Sequence Synthetic oligonucleotide
auggacugauagacaccaunn

SEQ ID NO: 127 - DNA, 21 bp, Artificial Sequence
  Description of Artificial Sequence Synthetic oligonucleotide
aacacuuuggucucuucuann

SEQ ID NO: 128 - DNA, 21 bp, Artificial Sequence
  Description of Artificial Sequence Synthetic oligonucleotide
uagaagagaccaaaguguunn

SEQ ID NO: 129 - DNA, 21 bp, Artificial Sequence
  Description of Artificial Sequence Synthetic oligonucleotide
agagaccagaucccugucunn

SEQ ID NO: 130 - DNA, 21 bp, Artificial Sequence
  Description of Artificial Sequence Synthetic oligonucleotide
agacagggaucuggucucunn

SEQ ID NO: 131 - DNA, 21 bp, Artificial Sequence
  Description of Artificial Sequence Synthetic oligonucleotide
accaugucugcccuaauuann

SEQ ID NO: 132 - DNA, 21 bp, Artificial Sequence
  Description of Artificial Sequence Synthetic oligonucleotide
uaauuagggcagacauggunn

SEQ ID NO: 133 - DNA, 21 bp, Artificial Sequence
  Description of Artificial Sequence Synthetic oligonucleotide
accucaguauuuucuauacnn

SEQ ID NO: 134 - DNA, 21 bp, Artificial Sequence
  Description of Artificial Sequence Synthetic oligonucleotide
guauagaaaauacugaggunn

SEQ ID NO: 135 - DNA, 21 bp, Artificial Sequence
  Description of Artificial Sequence Synthetic oligonucleotide
ugucuaucaguccaugaccnn

SEQ ID NO: 136 - DNA, 21 bp, Artificial Sequence
  Description of Artificial Sequence Synthetic oligonucleotide
ggucauggacugauagacann

SEQ ID NO: 137 - DNA, 21 bp, Artificial Sequence
  Description of Artificial Sequence Synthetic oligonucleotide
cauccggaccaagauguucnn

SEQ ID NO: 138 - DNA, 21 bp, Artificial Sequence
  Description of Artificial Sequence Synthetic oligonucleotide
gaacaucuugguccggaugnn

SEQ ID NO: 139 - DNA, 21 bp, Artificial Sequence
  Description of Artificial Sequence Synthetic oligonucleotide
ucucaaccucaguauuuucnn

SEQ ID NO: 140 - DNA, 21 bp, Artificial Sequence
  Description of Artificial Sequence Synthetic oligonucleotide
gaaaauacugagguugagann

SEQ ID NO: 141 - DNA, 21 bp, Artificial Sequence
  Description of Artificial Sequence Synthetic oligonucleotide
ucauccggaccaagauguunn

SEQ ID NO: 142 - DNA, 21 bp, Artificial Sequence
  Description of Artificial Sequence Synthetic oligonucleotide
aacaucuugguccggaugann

SEQ ID NO: 143 - DNA, 21 bp, Artificial Sequence
  Description of Artificial Sequence Synthetic oligonucleotide
aauggugucuaucaguccann

SEQ ID NO: 144 - DNA, 21 bp, Artificial Sequence
  Description of Artificial Sequence Synthetic oligonucleotide
uggacugauagacaccauunn

SEQ ID NO: 145 - DNA, 21 bp, Artificial Sequence
  Description of Artificial Sequence Synthetic oligonucleotide
ccucaguauuuucuauacann

SEQ ID NO: 146 - DNA, 21 bp, Artificial Sequence
  Description of Artificial Sequence Synthetic oligonucleotide
uguauagaaaauacugaggnn

SEQ ID NO: 147 - DNA, 21 bp, Artificial Sequence
  Description of Artificial Sequence Synthetic oligonucleotide
acuuuggucucuucuacgcnn

SEQ ID NO: 148 - DNA, 21 bp, Artificial Sequence
  Description of Artificial Sequence Synthetic oligonucleotide
gcguagaagagaccaaagunn

SEQ ID NO: 149 - DNA, 21 bp, Artificial Sequence
  Description of Artificial Sequence Synthetic oligonucleotide
cccucucaaccucaguauunn

SEQ ID NO: 150 - DNA, 21 bp, Artificial Sequence
  Description of Artificial Sequence Synthetic oligonucleotide
aauacugagguugagagggnn

SEQ ID NO: 151 - DNA, 21 bp, Artificial Sequence
  Description of Artificial Sequence Synthetic oligonucleotide
ccaaacacuuuggucucuunn

SEQ ID NO: 152 - DNA, 21 bp, Artificial Sequence
  Description of Artificial Sequence Synthetic oligonucleotide
aagagaccaaaguguuuggnn

SEQ ID NO: 153 - DNA, 21 bp, Artificial Sequence
  Description of Artificial Sequence Synthetic oligonucleotide
aaccucaguauuuucuauann

SEQ ID NO: 154 - DNA, 21 bp, Artificial Sequence
  Description of Artificial Sequence Synthetic oligonucleotide
uauagaaaauacugagguunn

SEQ ID NO: 155 - DNA, 21 bp, Artificial Sequence
  Description of Artificial Sequence Synthetic oligonucleotide
gucuaucaguccaugaccann

SEQ ID NO: 156 - DNA, 21 bp, Artificial Sequence
  Description of Artificial Sequence Synthetic oligonucleotide
uggucauggacugauagacnn

SEQ ID NO: 157 - DNA, 21 bp, Artificial Sequence
  Description of Artificial Sequence Synthetic oligonucleotide
ucagcaccgagaacaucuann

SEQ ID NO: 158 - DNA, 21 bp, Artificial Sequence
  Description of Artificial Sequence Synthetic oligonucleotide
uagauguucucggugcugann

SEQ ID NO: 159 - DNA, 21 bp, Artificial Sequence
  Description of Artificial Sequence Synthetic oligonucleotide
ggugucuaucaguccaugann

SEQ ID NO: 160 - DNA, 21 bp, Artificial Sequence
  Description of Artificial Sequence Synthetic oligonucleotide
ucauggacugauagacaccnn

SEQ ID NO: 161 - DNA, 21 bp, Artificial Sequence
  Description of Artificial Sequence Synthetic oligonucleotide
ccccucucaaccucaguaunn

SEQ ID NO: 162 - DNA, 21 bp, Artificial Sequence
  Description of Artificial Sequence Synthetic oligonucleotide
auacugagguugagaggggnn

SEQ ID NO: 163 - DNA, 21 bp, Artificial Sequence
  Description of Artificial Sequence Synthetic oligonucleotide
agcaccgagaacaucuacunn

SEQ ID NO: 164 - DNA, 21 bp, Artificial Sequence
  Description of Artificial Sequence Synthetic oligonucleotide
aguagauguucucggugcunn

SEQ ID NO: 165 - DNA, 21 bp, Artificial Sequence
  Description of Artificial Sequence Synthetic oligonucleotide
cagcaccgagaacaucuacnn

SEQ ID NO: 166 - DNA, 21 bp, Artificial Sequence
  Description of Artificial Sequence Synthetic oligonucleotide
guagauguucucggugcugnn

SEQ ID NO: 167 - DNA, 21 bp, Artificial Sequence
  Description of Artificial Sequence Synthetic oligonucleotide
auggugucuaucaguccaunn

SEQ ID NO: 168 - DNA, 21 bp, Artificial Sequence
  Description of Artificial Sequence Synthetic oligonucleotide
auggacugauagacaccaunn

SEQ ID NO: 169 - DNA, 21 bp, Artificial Sequence
  Description of Artificial Sequence Synthetic oligonucleotide
aacacuuuggucucuucuann

SEQ ID NO: 170 - DNA, 21 bp, Artificial Sequence
  Description of Artificial Sequence Synthetic oligonucleotide
uagaagagaccaaaguguunn

SEQ ID NO: 171 - DNA, 21 bp, Artificial Sequence
  Description of Artificial Sequence Synthetic oligonucleotide
agagaccagaucccugucunn

SEQ ID NO: 172 - DNA, 21 bp, Artificial Sequence
  Description of Artificial Sequence Synthetic oligonucleotide
agacagggaucuggucucunn

SEQ ID NO: 173 - DNA, 21 bp, Artificial Sequence
  Description of Artificial Sequence Synthetic oligonucleotide
accaugucugcccuaauuatt

SEQ ID NO: 174 - DNA, 21 bp, Artificial Sequence
  Description of Artificial Sequence Synthetic oligonucleotide
uaauuagggcagacauggutt

SEQ ID NO: 175 - DNA, 21 bp, Artificial Sequence
  Description of Artificial Sequence Synthetic oligonucleotide
accucaguauuuucuauactt

SEQ ID NO: 176 - DNA, 21 bp, Artificial Sequence
  Description of Artificial Sequence Synthetic oligonucleotide
guauagaaaauacugaggutt

SEQ ID NO: 177 - DNA, 21 bp, Artificial Sequence
  Description of Artificial Sequence Synthetic oligonucleotide
cucucaaccucaguauuuutt

SEQ ID NO: 178 - DNA, 21 bp, Artificial Sequence
  Description of Artificial Sequence Synthetic oligonucleotide
aaaauacugagguugagagtt

SEQ ID NO: 179 - DNA, 21 bp, Artificial Sequence
  Description of Artificial Sequence Synthetic oligonucleotide
ugucuaucaguccaugacctt

SEQ ID NO: 180 - DNA, 21 bp, Artificial Sequence
  Description of Artificial Sequence Synthetic oligonucleotide
ggucauggacugauagacatt

SEQ ID NO: 181 - DNA, 21 bp, Artificial Sequence
  Description of Artificial Sequence Synthetic oligonucleotide
cauccggaccaagauguuctt

SEQ ID NO: 182 - DNA, 21 bp, Artificial Sequence
  Description of Artificial Sequence Synthetic oligonucleotide
gaacaucuugguccggaugtt

SEQ ID NO: 183 - DNA, 21 bp, Artificial Sequence
  Description of Artificial Sequence Synthetic oligonucleotide
ucucaaccucaguauuuuctt

SEQ ID NO: 184 - DNA, 21 bp, Artificial Sequence
  Description of Artificial Sequence Synthetic oligonucleotide
gaaaauacugagguugagatt

SEQ ID NO: 185 - DNA, 21 bp, Artificial Sequence
  Description of Artificial Sequence Synthetic oligonucleotide
ucauccggaccaagauguutt

SEQ ID NO: 186 - DNA, 21 bp, Artificial Sequence
  Description of Artificial Sequence Synthetic oligonucleotide
aacaucuugguccggaugatt

SEQ ID NO: 187 - DNA, 21 bp, Artificial Sequence
  Description of Artificial Sequence Synthetic oligonucleotide
aauggugucuaucaguccatt

SEQ ID NO: 188 - DNA, 21 bp, Artificial Sequence
  Description of Artificial Sequence Synthetic oligonucleotide
uggacugauagacaccauutt

SEQ ID NO: 189 - DNA, 21 bp, Artificial Sequence
  Description of Artificial Sequence Synthetic oligonucleotide
ccucaguauuuucuauacatt

SEQ ID NO: 190 - DNA, 21 bp, Artificial Sequence
  Description of Artificial Sequence Synthetic oligonucleotide
uguauagaaaauacugaggtt

SEQ ID NO: 191 - DNA, 21 bp, Artificial Sequence
  Description of Artificial Sequence Synthetic oligonucleotide
acuuuggucucuucuacgctt

SEQ ID NO: 192 - DNA, 21 bp, Artificial Sequence
  Description of Artificial Sequence Synthetic oligonucleotide
gcguagaagagaccaaagutt

SEQ ID NO: 193 - DNA, 21 bp, Artificial Sequence
  Description of Artificial Sequence Synthetic oligonucleotide
cccucucaaccucaguauutt

SEQ ID NO: 194 - DNA, 21 bp, Artificial Sequence
  Description of Artificial Sequence Synthetic oligonucleotide
aauacugagguugagagggtt

SEQ ID NO: 195 - DNA, 21 bp, Artificial Sequence
  Description of Artificial Sequence Synthetic oligonucleotide
ccaaacacuuuggucucuutt

SEQ ID NO: 196 - DNA, 21 bp, Artificial Sequence
  Description of Artificial Sequence Synthetic oligonucleotide
aagagaccaaaguguuuggtt

SEQ ID NO: 197 - DNA, 21 bp, Artificial Sequence
  Description of Artificial Sequence Synthetic oligonucleotide
aaccucaguauuuucuauatt

SEQ ID NO: 198 - DNA, 21 bp, Artificial Sequence
  Description of Artificial Sequence Synthetic oligonucleotide
uauagaaaauacugagguutt

SEQ ID NO: 199 - DNA, 21 bp, Artificial Sequence
  Description of Artificial Sequence Synthetic oligonucleotide
gucuaucaguccaugaccatt

SEQ ID NO: 200 - DNA, 21 bp, Artificial Sequence
  Description of Artificial Sequence Synthetic oligonucleotide
uggucauggacugauagactt

SEQ ID NO: 201 - DNA, 21 bp, Artificial Sequence
  Description of Artificial Sequence Synthetic oligonucleotide
ucagcaccgagaacaucuatt

SEQ ID NO: 202 - DNA, 21 bp, Artificial Sequence
  Description of Artificial Sequence Synthetic oligonucleotide
uagauguucucggugcugatt

SEQ ID NO: 203 - DNA, 21 bp, Artificial Sequence
  Description of Artificial Sequence Synthetic oligonucleotide
ggugucuaucaguccaugatt

SEQ ID NO: 204 - DNA, 21 bp, Artificial Sequence
  Description of Artificial Sequence Synthetic oligonucleotide
ucauggacugauagacacctt

SEQ ID NO: 205 - DNA, 21 bp, Artificial Sequence
  Description of Artificial Sequence Synthetic oligonucleotide
ccccucucaaccucaguautt

SEQ ID NO: 206 - DNA, 21 bp, Artificial Sequence
  Description of Artificial Sequence Synthetic oligonucleotide
auacugagguugagaggggtt

SEQ ID NO: 207 - DNA, 21 bp, Artificial Sequence
  Description of Artificial Sequence Synthetic oligonucleotide
agcaccgagaacaucuacutt

SEQ ID NO: 208 - DNA, 21 bp, Artificial Sequence
  Description of Artificial Sequence Synthetic oligonucleotide
aguagauguucucggugcutt

SEQ ID NO: 209 - DNA, 21 bp, Artificial Sequence
  Description of Artificial Sequence Synthetic oligonucleotide
cagcaccgagaacaucuactt

SEQ ID NO: 210 - DNA, 21 bp, Artificial Sequence
  Description of Artificial Sequence Synthetic oligonucleotide
guagauguucucggugcugtt

SEQ ID NO: 211 - DNA, 21 bp, Artificial Sequence
  Description of Artificial Sequence Synthetic oligonucleotide
auggugucuaucaguccautt

SEQ ID NO: 212 - DNA, 21 bp, Artificial Sequence
  Description of Artificial Sequence Synthetic oligonucleotide
auggacugauagacaccautt

SEQ ID NO: 213 - DNA, 21 bp, Artificial Sequence
  Description of Artificial Sequence Synthetic oligonucleotide
aacacuuuggucucuucuatt

SEQ ID NO: 214 - DNA, 21 bp, Artificial Sequence
  Description of Artificial Sequence Synthetic oligonucleotide
uagaagagaccaaaguguutt

SEQ ID NO: 215 - DNA, 21 bp, Artificial Sequence
  Description of Artificial Sequence Synthetic oligonucleotide
agagaccagaucccugucutt

SEQ ID NO: 216 - DNA, 21 bp, Artificial Sequence
  Description of Artificial Sequence Synthetic oligonucleotide
agacagggaucuggucucutt

SEQ ID NO: 217 - DNA, 21 bp, Artificial Sequence
  Description of Artificial Sequence Synthetic oligonucleotide
accaugucugcccuaauuatt

SEQ ID NO: 218 - DNA, 21 bp, Artificial Sequence
  Description of Artificial Sequence Synthetic oligonucleotide
uaauuagggcagacauggutt

SEQ ID NO: 219 - DNA, 21 bp, Artificial Sequence
  Description of Artificial Sequence Synthetic oligonucleotide
accucaguauuuucuauactt

SEQ ID NO: 220 - DNA, 21 bp, Artificial Sequence
  Description of Artificial Sequence Synthetic oligonucleotide
guauagaaaauacugaggutt

SEQ ID NO: 221 - DNA, 21 bp, Artificial Sequence
  Description of Artificial Sequence Synthetic oligonucleotide
ugucuaucaguccaugacctt

SEQ ID NO: 222 - DNA, 21 bp, Artificial Sequence
  Description of Artificial Sequence Synthetic oligonucleotide
ggucauggacugauagacatt

SEQ ID NO: 223 - DNA, 21 bp, Artificial Sequence
  Description of Artificial Sequence Synthetic oligonucleotide
cauccggaccaagauguuctt

SEQ ID NO: 224 - DNA, 21 bp, Artificial Sequence
  Description of Artificial Sequence Synthetic oligonucleotide
gaacaucuugguccggaugtt

SEQ ID NO: 225 - DNA, 21 bp, Artificial Sequence
  Description of Artificial Sequence Synthetic oligonucleotide
ucucaaccucaguauuuuctt

SEQ ID NO: 226 - DNA, 21 bp, Artificial Sequence
  Description of Artificial Sequence Synthetic oligonucleotide
gaaaauacugagguugagatt

SEQ ID NO: 227 - DNA, 21 bp, Artificial Sequence
  Description of Artificial Sequence Synthetic oligonucleotide
ucauccggaccaagauguutt

SEQ ID NO: 228 - DNA, 21 bp, Artificial Sequence
  Description of Artificial Sequence Synthetic oligonucleotide
aacaucuugguccggaugatt

SEQ ID NO: 229 - DNA, 21 bp, Artificial Sequence
  Description of Artificial Sequence Synthetic oligonucleotide
aauggugucuaucaguccatt

SEQ ID NO: 230 - DNA, 21 bp, Artificial Sequence
  Description of Artificial Sequence Synthetic oligonucleotide
uggacugauagacaccauutt

SEQ ID NO: 231 - DNA, 21 bp, Artificial Sequence
  Description of Artificial Sequence Synthetic oligonucleotide
ccucaguauuuucuauacatt

SEQ ID NO: 232 - DNA, 21 bp, Artificial Sequence
  Description of Artificial Sequence Synthetic oligonucleotide
uguauagaaaauacugaggtt

SEQ ID NO: 233 - DNA, 21 bp, Artificial Sequence
  Description of Artificial Sequence Synthetic oligonucleotide
acuuuggucucuucuacgctt

SEQ ID NO: 234 - DNA, 21 bp, Artificial Sequence
  Description of Artificial Sequence Synthetic oligonucleotide
gcguagaagagaccaaagutt

SEQ ID NO: 235 - DNA, 21 bp, Artificial Sequence
  Description of Artificial Sequence Synthetic oligonucleotide
cccucucaaccucaguauutt

SEQ ID NO: 236 - DNA, 21 bp, Artificial Sequence
  Description of Artificial Sequence Synthetic oligonucleotide
aauacugagguugagagggtt

SEQ ID NO: 237 - DNA, 21 bp, Artificial Sequence
  Description of Artificial Sequence Synthetic oligonucleotide
ccaaacacuuuggucucuutt

SEQ ID NO: 238 - DNA, 21 bp, Artificial Sequence
  Description of Artificial Sequence Synthetic oligonucleotide
aagagaccaaaguguuuggtt

SEQ ID NO: 239 - DNA, 21 bp, Artificial Sequence
  Description of Artificial Sequence Synthetic oligonucleotide
aaccucaguauuuucuauatt

SEQ ID NO: 240 - DNA, 21 bp, Artificial Sequence
  Description of Artificial Sequence Synthetic oligonucleotide
uauagaaaauacugagguutt

SEQ ID NO: 241 - DNA, 21 bp, Artificial Sequence
  Description of Artificial Sequence Synthetic oligonucleotide
gucuaucaguccaugaccatt

SEQ ID NO: 242 - DNA, 21 bp, Artificial Sequence
  Description of Artificial Sequence Synthetic oligonucleotide
uggucauggacugauagactt

SEQ ID NO: 243 - DNA, 21 bp, Artificial Sequence
  Description of Artificial Sequence Synthetic oligonucleotide
ucagcaccgagaacaucuatt

SEQ ID NO: 244 - DNA, 21 bp, Artificial Sequence
  Description of Artificial Sequence Synthetic oligonucleotide
uagauguucucggugcugatt

SEQ ID NO: 245 - DNA, 21 bp, Artificial Sequence
  Description of Artificial Sequence Synthetic oligonucleotide
ggugucuaucaguccaugatt

SEQ ID NO: 246 - DNA, 21 bp, Artificial Sequence
  Description of Artificial Sequence Synthetic oligonucleotide
ucauggacugauagacacctt

SEQ ID NO: 247 - DNA, 21 bp, Artificial Sequence
  Description of Artificial Sequence Synthetic oligonucleotide
ccccucucaaccucaguautt

SEQ ID NO: 248 - DNA, 21 bp, Artificial Sequence
  Description of Artificial Sequence Synthetic oligonucleotide
auacugagguugagaggggtt

SEQ ID NO: 249 - DNA, 21 bp, Artificial Sequence
  Description of Artificial Sequence Synthetic oligonucleotide
agcaccgagaacaucuacutt

SEQ ID NO: 250 - DNA, 21 bp, Artificial Sequence
  Description of Artificial Sequence Synthetic oligonucleotide
aguagauguucucggugcutt

SEQ ID NO: 251 - DNA, 21 bp, Artificial Sequence
  Description of Artificial Sequence Synthetic oligonucleotide
cagcaccgagaacaucuactt

SEQ ID NO: 252 - DNA, 21 bp, Artificial Sequence
  Description of Artificial Sequence Synthetic oligonucleotide
guagauguucucggugcugtt

SEQ ID NO: 253 - DNA, 21 bp, Artificial Sequence
  Description of Artificial Sequence Synthetic oligonucleotide
auggugucuaucaguccautt

SEQ ID NO: 254 - DNA, 21 bp, Artificial Sequence
  Description of Artificial Sequence Synthetic oligonucleotide
auggacugauagacaccautt

SEQ ID NO: 255 - DNA, 21 bp, Artificial Sequence
  Description of Artificial Sequence Synthetic oligonucleotide
aacacuuuggucucuucuatt

SEQ ID NO: 256 - DNA, 21 bp, Artificial Sequence
  Description of Artificial Sequence Synthetic oligonucleotide
uagaagagaccaaaguguutt

SEQ ID NO: 257 - DNA, 21 bp, Artificial Sequence
  Description of Artificial Sequence Synthetic oligonucleotide
agagaccagaucccugucutt

SEQ ID NO: 258 - DNA, 21 bp, Artificial Sequence
  Description of Artificial Sequence Synthetic oligonucleotide
agacagggaucuggucucutt

SEQ ID NO: 259 - DNA, 21 bp, Artificial Sequence
  Description of Artificial Sequence Synthetic oligonucleotide
accaugucugcccuaauuatt

SEQ ID NO: 260 - DNA, 21 bp, Artificial Sequence
  Description of Artificial Sequence Synthetic oligonucleotide
uaauuagggcagacauggutt

SEQ ID NO: 261 - DNA, 21 bp, Artificial Sequence
  Description of Artificial Sequence Synthetic oligonucleotide
accucaguauuuucuauactt

SEQ ID NO: 262 - DNA, 21 bp, Artificial Sequence
  Description of Artificial Sequence Synthetic oligonucleotide
guauagaaaauacugaggutt

SEQ ID NO: 263 - DNA, 21 bp, Artificial Sequence
  Description of Artificial Sequence Synthetic oligonucleotide
cucucaaccucaguauuuutt

SEQ ID NO: 264 - DNA, 21 bp, Artificial Sequence
  Description of Artificial Sequence Synthetic oligonucleotide
aaaauacugagguugagagtt

SEQ ID NO: 265 - DNA, 21 bp, Artificial Sequence
  Description of Artificial Sequence Synthetic oligonucleotide
ugucuaucaguccaugacctt

SEQ ID NO: 266 - DNA, 21 bp, Artificial Sequence
  Description of Artificial Sequence Synthetic oligonucleotide
ggucauggacugauagacatt

SEQ ID NO: 267 - DNA, 21 bp, Artificial Sequence
  Description of Artificial Sequence Synthetic oligonucleotide
cauccggaccaagauguuctt

SEQ ID NO: 268 - DNA, 21 bp, Artificial Sequence
  Description of Artificial Sequence Synthetic oligonucleotide
gaacaucuugguccggaugtt

SEQ ID NO: 269 - DNA, 21 bp, Artificial Sequence
  Description of Artificial Sequence Synthetic oligonucleotide
ucucaaccucaguauuuuctt

SEQ ID NO: 270 - DNA, 21 bp, Artificial Sequence
  Description of Artificial Sequence Synthetic oligonucleotide
gaaaauacugagguugagatt

SEQ ID NO: 271 - DNA, 21 bp, Artificial Sequence
  Description of Artificial Sequence Synthetic oligonucleotide
ucauccggaccaagauguutt

SEQ ID NO: 272 - DNA, 21 bp, Artificial Sequence
  Description of Artificial Sequence Synthetic oligonucleotide
aacaucuugguccggaugatt

SEQ ID NO: 273 - DNA, 21 bp, Artificial Sequence
  Description of Artificial Sequence Synthetic oligonucleotide
aauggugucuaucaguccatt

SEQ ID NO: 274 - DNA, 21 bp, Artificial Sequence
  Description of Artificial Sequence Synthetic oligonucleotide
uggacugauagacaccauutt

SEQ ID NO: 275 - DNA, 21 bp, Artificial Sequence
  Description of Artificial Sequence Synthetic oligonucleotide
ccucaguauuuucuauacatt

SEQ ID NO: 276 - DNA, 21 bp, Artificial Sequence
  Description of Artificial Sequence Synthetic oligonucleotide
uguauagaaaauacugaggtt

SEQ ID NO: 277 - DNA, 21 bp, Artificial Sequence
  Description of Artificial Sequence Synthetic oligonucleotide
acuuuggucucuucuacgctt

SEQ ID NO: 278 - DNA, 21 bp, Artificial Sequence
  Description of Artificial Sequence Synthetic oligonucleotide
gcguagaagagaccaaagutt

SEQ ID NO: 279 - DNA, 21 bp, Artificial Sequence
  Description of Artificial Sequence Synthetic oligonucleotide
cccucucaaccucaguauutt

SEQ ID NO: 280 - DNA, 21 bp, Artificial Sequence
  Description of Artificial Sequence Synthetic oligonucleotide
aauacugagguugagagggtt

SEQ ID NO: 281 - DNA, 21 bp, Artificial Sequence
  Description of Artificial Sequence Synthetic oligonucleotide
ccaaacacuuuggucucuutt

SEQ ID NO: 282 - DNA, 21 bp, Artificial Sequence
  Description of Artificial Sequence Synthetic oligonucleotide
aagagaccaaaguguuuggtt

SEQ ID NO: 283 - DNA, 21 bp, Artificial Sequence
  Description of Artificial Sequence Synthetic oligonucleotide
aaccucaguauuuucuauatt

SEQ ID NO: 284 - DNA, 21 bp, Artificial Sequence
  Description of Artificial Sequence Synthetic oligonucleotide
uauagaaaauacugagguutt

SEQ ID NO: 285 - DNA, 21 bp, Artificial Sequence
  Description of Artificial Sequence Synthetic oligonucleotide
gucuaucaguccaugaccatt

SEQ ID NO: 286 - DNA, 21 bp, Artificial Sequence
  Description of Artificial Sequence Synthetic oligonucleotide
uggucauggacugauagactt

SEQ ID NO: 287 - DNA, 21 bp, Artificial Sequence
  Description of Artificial Sequence Synthetic oligonucleotide
ucagcaccgagaacaucuatt

SEQ ID NO: 288 - DNA, 21 bp, Artificial Sequence
  Description of Artificial Sequence Synthetic oligonucleotide
uagauguucucggugcugatt

SEQ ID NO: 289 - DNA, 21 bp, Artificial Sequence
  Description of Artificial Sequence Synthetic oligonucleotide
ggugucuaucaguccaugatt

SEQ ID NO: 290 - DNA, 21 bp, Artificial Sequence
  Description of Artificial Sequence Synthetic oligonucleotide
ucauggacugauagacacctt

SEQ ID NO: 291 - DNA, 21 bp, Artificial Sequence
  Description of Artificial Sequence Synthetic oligonucleotide
ccccucucaaccucaguautt

SEQ ID NO: 292 - DNA, 21 bp, Artificial Sequence
  Description of Artificial Sequence Synthetic oligonucleotide
auacugagguugagaggggtt

SEQ ID NO: 293 - DNA, 21 bp, Artificial Sequence
  Description of Artificial Sequence Synthetic oligonucleotide
agcaccgagaacaucuacutt

SEQ ID NO: 294 - DNA, 21 bp, Artificial Sequence
  Description of Artificial Sequence Synthetic oligonucleotide
aguagauguucucggugcutt

SEQ ID NO: 295 - DNA, 21 bp, Artificial Sequence
  Description of Artificial Sequence Synthetic oligonucleotide
cagcaccgagaacaucuactt

SEQ ID NO: 296 - DNA, 21 bp, Artificial Sequence
  Description of Artificial Sequence Synthetic oligonucleotide
guagauguucucggugcugtt

SEQ ID NO: 297 - DNA, 21 bp, Artificial Sequence
  Description of Artificial Sequence Synthetic oligonucleotide
auggugucuaucaguccautt

SEQ ID NO: 298 - DNA, 21 bp, Artificial Sequence
  Description of Artificial Sequence Synthetic oligonucleotide
auggacugauagacaccautt

SEQ ID NO: 299 - DNA, 21 bp, Artificial Sequence
  Description of Artificial Sequence Synthetic oligonucleotide
aacacuuuggucucuucuatt

SEQ ID NO: 300 - DNA, 21 bp, Artificial Sequence
  Description of Artificial Sequence Synthetic oligonucleotide
uagaagagaccaaaguguutt

SEQ ID NO: 301 - DNA, 21 bp, Artificial Sequence
  Description of Artificial Sequence Synthetic oligonucleotide
agagaccagaucccugucutt

SEQ ID NO: 302 - DNA, 21 bp, Artificial Sequence
  Description of Artificial Sequence Synthetic oligonucleotide
agacagggaucuggucucutt

SEQ ID NO: 303 - DNA, 21 bp, Artificial Sequence
  Description of Artificial Sequence Synthetic oligonucleotide
accaugucugcccuaauuatt

SEQ ID NO: 304 - DNA, 21 bp, Artificial Sequence
  Description of Artificial Sequence Synthetic oligonucleotide
uaauuagggcagacauggutt

SEQ ID NO: 305 - DNA, 21 bp, Artificial Sequence
  Description of Artificial Sequence Synthetic oligonucleotide
accucaguauuuucuauactt

SEQ ID NO: 306 - DNA, 21 bp, Artificial Sequence
  Description of Artificial Sequence Synthetic oligonucleotide
guauagaaaauacugaggutt

SEQ ID NO: 307 - DNA, 21 bp, Artificial Sequence
  Description of Artificial Sequence Synthetic oligonucleotide
ugucuaucaguccaugacctt

SEQ ID NO: 308 - DNA, 21 bp, Artificial Sequence
  Description of Artificial Sequence Synthetic oligonucleotide
ggucauggacugauagacatt

SEQ ID NO: 309 - DNA, 21 bp, Artificial Sequence
  Description of Artificial Sequence Synthetic oligonucleotide
cauccggaccaagauguuctt

SEQ ID NO: 310 - DNA, 21 bp, Artificial Sequence
  Description of Artificial Sequence Synthetic oligonucleotide
gaacaucuugguccggaugtt

SEQ ID NO: 311 - DNA, 21 bp, Artificial Sequence
  Description of Artificial Sequence Synthetic oligonucleotide
ucucaaccucaguauuuuctt

SEQ ID NO: 312 - DNA, 21 bp, Artificial Sequence
  Description of Artificial Sequence Synthetic oligonucleotide
gaaaauacugagguugagatt

SEQ ID NO: 313 - DNA, 21 bp, Artificial Sequence
  Description of Artificial Sequence Synthetic oligonucleotide
ucauccggaccaagauguutt

SEQ ID NO: 314 - DNA, 21 bp, Artificial Sequence
  Description of Artificial Sequence Synthetic oligonucleotide
aacaucuugguccggaugatt

SEQ ID NO: 315 - DNA, 21 bp, Artificial Sequence
  Description of Artificial Sequence Synthetic oligonucleotide
aauggugucuaucaguccatt

SEQ ID NO: 316 - DNA, 21 bp, Artificial Sequence
  Description of Artificial Sequence Synthetic oligonucleotide
uggacugauagacaccauutt

SEQ ID NO: 317 - DNA, 21 bp, Artificial Sequence
  Description of Artificial Sequence Synthetic oligonucleotide
ccucaguauuuucuauacatt

SEQ ID NO: 318 - DNA, 21 bp, Artificial Sequence
  Description of Artificial Sequence Synthetic oligonucleotide
uguauagaaaauacugaggtt

SEQ ID NO: 319 - DNA, 21 bp, Artificial Sequence
  Description of Artificial Sequence Synthetic oligonucleotide
acuuuggucucuucuacgctt

SEQ ID NO: 320 - DNA, 21 bp, Artificial Sequence
  Description of Artificial Sequence Synthetic oligonucleotide
gcguagaagagaccaaagutt

SEQ ID NO: 321 - DNA, 21 bp, Artificial Sequence
  Description of Artificial Sequence Synthetic oligonucleotide
cccucucaaccucaguauutt

SEQ ID NO: 322 - DNA, 21 bp, Artificial Sequence
  Description of Artificial Sequence Synthetic oligonucleotide
aauacugagguugagagggtt

SEQ ID NO: 323 - DNA, 21 bp, Artificial Sequence
  Description of Artificial Sequence Synthetic oligonucleotide
ccaaacacuuuggucucuutt

SEQ ID NO: 324 - DNA, 21 bp, Artificial Sequence
  Description of Artificial Sequence Synthetic oligonucleotide
aagagaccaaaguguuuggtt

SEQ ID NO: 325 - DNA, 21 bp, Artificial Sequence
  Description of Artificial Sequence Synthetic oligonucleotide
aaccucaguauuuucuauatt

SEQ ID NO: 326 - DNA, 21 bp, Artificial Sequence
  Description of Artificial Sequence Synthetic oligonucleotide
uauagaaaauacugagguutt

SEQ ID NO: 327 - DNA, 21 bp, Artificial Sequence
  Description of Artificial Sequence Synthetic oligonucleotide
gucuaucaguccaugaccatt

SEQ ID NO: 328 - DNA, 21 bp, Artificial Sequence
  Description of Artificial Sequence Synthetic oligonucleotide
uggucauggacugauagactt

SEQ ID NO: 329 - DNA, 21 bp, Artificial Sequence
  Description of Artificial Sequence Synthetic oligonucleotide
ucagcaccgagaacaucuatt

SEQ ID NO: 330 - DNA, 21 bp, Artificial Sequence
  Description of Artificial Sequence Synthetic oligonucleotide
uagauguucucggugcugatt

SEQ ID NO: 331 - DNA, 21 bp, Artificial Sequence
  Description of Artificial Sequence Synthetic oligonucleotide
ggugucuaucaguccaugatt

SEQ ID NO: 332 - DNA, 21 bp, Artificial Sequence
  Description of Artificial Sequence Synthetic oligonucleotide
ucauggacugauagacacctt

SEQ ID NO: 333 - DNA, 21 bp, Artificial Sequence
  Description of Artificial Sequence Synthetic oligonucleotide
ccccucucaaccucaguautt

SEQ ID NO: 334 - DNA, 21 bp, Artificial Sequence
  Description of Artificial Sequence Synthetic oligonucleotide
auacugagguugagaggggtt

SEQ ID NO: 335 - DNA, 21 bp, Artificial Sequence
  Description of Artificial Sequence Synthetic oligonucleotide
agcaccgagaacaucuacutt

SEQ ID NO: 336 - DNA, 21 bp, Artificial Sequence
  Description of Artificial Sequence Synthetic oligonucleotide
aguagauguucucggugcutt

SEQ ID NO: 337 - DNA, 21 bp, Artificial Sequence
  Description of Artificial Sequence Synthetic oligonucleotide
cagcaccgagaacaucuactt

SEQ ID NO: 338 - DNA, 21 bp, Artificial Sequence
  Description of Artificial Sequence Synthetic oligonucleotide
guagauguucucggugcugtt

SEQ ID NO: 339 - DNA, 21 bp, Artificial Sequence
  Description of Artificial Sequence Synthetic oligonucleotide
auggugucuaucaguccautt

SEQ ID NO: 340 - DNA, 21 bp, Artificial Sequence
  Description of Artificial Sequence Synthetic oligonucleotide
auggacugauagacaccautt

SEQ ID NO: 341 - DNA, 21 bp, Artificial Sequence
  Description of Artificial Sequence Synthetic oligonucleotide
aacacuuuggucucuucuatt

SEQ ID NO: 342 - DNA, 21 bp, Artificial Sequence
  Description of Artificial Sequence Synthetic oligonucleotide
uagaagagaccaaaguguutt

SEQ ID NO: 343 - DNA, 21 bp, Artificial Sequence
  Description of Artificial Sequence Synthetic oligonucleotide
agagaccagaucccugucutt

SEQ ID NO: 344 - DNA, 21 bp, Artificial Sequence
  Description of Artificial Sequence Synthetic oligonucleotide
agacagggaucuggucucutt

SEQ ID NO: 345 - DNA, 21 bp, Artificial Sequence
  Description of Artificial Sequence Synthetic oligonucleotide
accaugucugcccuaauuatt

SEQ ID NO: 346 - DNA, 21 bp, Artificial Sequence
  Description of Artificial Sequence Synthetic oligonucleotide
uaauuagggcagacauggutt

SEQ ID NO: 347 - DNA, 21 bp, Artificial Sequence
  Description of Artificial Sequence Synthetic oligonucleotide
accucaguauuuucuauactt

SEQ ID NO: 348 - DNA, 21 bp, Artificial Sequence
  Description of Artificial Sequence Synthetic oligonucleotide
guauagaaaauacugaggutt

SEQ ID NO: 349 - DNA, 21 bp, Artificial Sequence
  Description of Artificial Sequence Synthetic oligonucleotide
cucucaaccucaguauuuutt

SEQ ID NO: 350 - DNA, 21 bp, Artificial Sequence
  Description of Artificial Sequence Synthetic oligonucleotide
aaaauacugagguugagagtt

SEQ ID NO: 351 - DNA, 21 bp, Artificial Sequence
  Description of Artificial Sequence Synthetic oligonucleotide
ugucuaucaguccaugacctt

SEQ ID NO: 352 - DNA, 21 bp, Artificial Sequence
  Description of Artificial Sequence Synthetic oligonucleotide
ggucauggacugauagacatt

SEQ ID NO: 353 - DNA, 21 bp, Artificial Sequence
  Description of Artificial Sequence Synthetic oligonucleotide
cauccggaccaagauguuctt

SEQ ID NO: 354 - DNA, 21 bp, Artificial Sequence
  Description of Artificial Sequence Synthetic oligonucleotide
gaacaucuugguccggaugtt

SEQ ID NO: 355 - DNA, 21 bp, Artificial Sequence
  Description of Artificial Sequence Synthetic oligonucleotide
ucucaaccucaguauuuuctt

SEQ ID NO: 356 - DNA, 21 bp, Artificial Sequence
  Description of Artificial Sequence Synthetic oligonucleotide
gaaaauacugagguugagatt

SEQ ID NO: 357 - DNA, 21 bp, Artificial Sequence
  Description of Artificial Sequence Synthetic oligonucleotide
ucauccggaccaagauguutt

SEQ ID NO: 358 - DNA, 21 bp, Artificial Sequence
  Description of Artificial Sequence Synthetic oligonucleotide
aacaucuugguccggaugatt

SEQ ID NO: 359 - DNA, 21 bp, Artificial Sequence
  Description of Artificial Sequence Synthetic oligonucleotide
aauggugucuaucaguccatt

SEQ ID NO: 360 - DNA, 21 bp, Artificial Sequence
  Description of Artificial Sequence Synthetic oligonucleotide
uggacugauagacaccauutt

SEQ ID NO: 361 - DNA, 21 bp, Artificial Sequence
  Description of Artificial Sequence Synthetic oligonucleotide
ccucaguauuuucuauacatt

SEQ ID NO: 362 - DNA, 21 bp, Artificial Sequence
  Description of Artificial Sequence Synthetic oligonucleotide
uguauagaaaauacugaggtt

SEQ ID NO: 363 - DNA, 21 bp, Artificial Sequence
  Description of Artificial Sequence Synthetic oligonucleotide
acuuuggucucuucuacgctt

SEQ ID NO: 364 - DNA, 21 bp, Artificial Sequence
  Description of Artificial Sequence Synthetic oligonucleotide
gcguagaagagaccaaagutt

SEQ ID NO: 365 - DNA, 21 bp, Artificial Sequence
  Description of Artificial Sequence Synthetic oligonucleotide
cccucucaaccucaguauutt

SEQ ID NO: 366 - DNA, 21 bp, Artificial Sequence
  Description of Artificial Sequence Synthetic oligonucleotide
aauacugagguugagagggtt

SEQ ID NO: 367 - DNA, 21 bp, Artificial Sequence
  Description of Artificial Sequence Synthetic oligonucleotide
ccaaacacuuuggucucuutt

SEQ ID NO: 368 - DNA, 21 bp, Artificial Sequence
  Description of Artificial Sequence Synthetic oligonucleotide
aagagaccaaaguguuuggtt

SEQ ID NO: 369 - DNA, 21 bp, Artificial Sequence
  Description of Artificial Sequence Synthetic oligonucleotide
aaccucaguauuuucuauatt

SEQ ID NO: 370 - DNA, 21 bp, Artificial Sequence
  Description of Artificial Sequence Synthetic oligonucleotide
uauagaaaauacugagguutt

SEQ ID NO: 371 - DNA, 21 bp, Artificial Sequence
  Description of Artificial Sequence Synthetic oligonucleotide
gucuaucaguccaugaccatt

SEQ ID NO: 372 - DNA, 21 bp, Artificial Sequence
  Description of Artificial Sequence Synthetic oligonucleotide
uggucauggacugauagactt

SEQ ID NO: 373 - DNA, 21 bp, Artificial Sequence
  Description of Artificial Sequence Synthetic oligonucleotide
ucagcaccgagaacaucuatt

SEQ ID NO: 374 - DNA, 21 bp, Artificial Sequence
  Description of Artificial Sequence Synthetic oligonucleotide
uagauguucucggugcugatt

SEQ ID NO: 375 - DNA, 21 bp, Artificial Sequence
  Description of Artificial Sequence Synthetic oligonucleotide
ggugucuaucaguccaugatt

SEQ ID NO: 376 - DNA, 21 bp, Artificial Sequence
  Description of Artificial Sequence Synthetic oligonucleotide
ucauggacugauagacacctt

SEQ ID NO: 377 - DNA, 21 bp, Artificial Sequence
  Description of Artificial Sequence Synthetic oligonucleotide
ccccucucaaccucaguautt

SEQ ID NO: 378 - DNA, 21 bp, Artificial Sequence
  Description of Artificial Sequence Synthetic oligonucleotide
auacugagguugagaggggtt

SEQ ID NO: 379 - DNA, 21 bp, Artificial Sequence
  Description of Artificial Sequence Synthetic oligonucleotide
agcaccgagaacaucuacutt

SEQ ID NO: 380 - DNA, 21 bp, Artificial Sequence
  Description of Artificial Sequence Synthetic oligonucleotide
aguagauguucucggugcutt

SEQ ID NO: 381 - DNA, 21 bp, Artificial Sequence
  Description of Artificial Sequence Synthetic oligonucleotide
cagcaccgagaacaucuactt

SEQ ID NO: 382 - DNA, 21 bp, Artificial Sequence
  Description of Artificial Sequence Synthetic oligonucleotide
guagauguucucggugcugtt

SEQ ID NO: 383 - DNA, 21 bp, Artificial Sequence
  Description of Artificial Sequence Synthetic oligonucleotide
auggugucuaucaguccautt

SEQ ID NO: 384 - DNA, 21 bp, Artificial Sequence
  Description of Artificial Sequence Synthetic oligonucleotide
auggacugauagacaccautt

SEQ ID NO: 385 - DNA, 21 bp, Artificial Sequence
  Description of Artificial Sequence Synthetic oligonucleotide
aacacuuuggucucuucuatt

SEQ ID NO: 386 - DNA, 21 bp, Artificial Sequence
  Description of Artificial Sequence Synthetic oligonucleotide
uagaagagaccaaaguguutt

SEQ ID NO: 387 - DNA, 21 bp, Artificial Sequence
  Description of Artificial Sequence Synthetic oligonucleotide
agagaccagaucccugucutt

SEQ ID NO: 388 - DNA, 21 bp, Artificial Sequence
  Description of Artificial Sequence Synthetic oligonucleotide
agacagggaucuggucucutt

SEQ ID NO: 389 - DNA, 21 bp, Artificial Sequence
  Description of Artificial Sequence Synthetic oligonucleotide
accaugucugcccuaauuatt

SEQ ID NO: 390 - DNA, 21 bp, Artificial Sequence
  Description of Artificial Sequence Synthetic oligonucleotide
uaauuagggcagacauggutt

SEQ ID NO: 391 - DNA, 21 bp, Artificial Sequence
  Description of Artificial Sequence Synthetic oligonucleotide
accucaguauuuucuauactt

SEQ ID NO: 392 - DNA, 21 bp, Artificial Sequence
  Description of Artificial Sequence Synthetic oligonucleotide
guauagaaaauacugaggutt

SEQ ID NO: 393 - DNA, 21 bp, Artificial Sequence
  Description of Artificial Sequence Synthetic oligonucleotide
ugucuaucaguccaugacctt

SEQ ID NO: 394 - DNA, 21 bp, Artificial Sequence
  Description of Artificial Sequence Synthetic oligonucleotide
ggucauggacugauagacatt

SEQ ID NO: 395 - DNA, 21 bp, Artificial Sequence
  Description of Artificial Sequence Synthetic oligonucleotide
cauccggaccaagauguuctt

SEQ ID NO: 396 - DNA, 21 bp, Artificial Sequence
  Description of Artificial Sequence Synthetic oligonucleotide
gaacaucuugguccggaugtt

SEQ ID NO: 397 - DNA, 21 bp, Artificial Sequence
  Description of Artificial Sequence Synthetic oligonucleotide
ucucaaccucaguauuuuctt

SEQ ID NO: 398 - DNA, 21 bp, Artificial Sequence
  Description of Artificial Sequence Synthetic oligonucleotide
gaaaauacugagguugagatt

SEQ ID NO: 399 - DNA, 21 bp, Artificial Sequence
  Description of Artificial Sequence Synthetic oligonucleotide
ucauccggaccaagauguutt

SEQ ID NO: 400 - DNA, 21 bp, Artificial Sequence
  Description of Artificial Sequence Synthetic oligonucleotide
aacaucuugguccggaugatt

SEQ ID NO: 401 - DNA, 21 bp, Artificial Sequence
  Description of Artificial Sequence Synthetic oligonucleotide
aauggugucuaucaguccatt

SEQ ID NO: 402 - DNA, 21 bp, Artificial Sequence
  Description of Artificial Sequence Synthetic oligonucleotide
uggacugauagacaccauutt

SEQ ID NO: 403 - DNA, 21 bp, Artificial Sequence
  Description of Artificial Sequence Synthetic oligonucleotide
ccucaguauuuucuauacatt

SEQ ID NO: 404 - DNA, 21 bp, Artificial Sequence
  Description of Artificial Sequence Synthetic oligonucleotide
uguauagaaaauacugaggtt

SEQ ID NO: 405 - DNA, 21 bp, Artificial Sequence
  Description of Artificial Sequence Synthetic oligonucleotide
acuuuggucucuucuacgctt

SEQ ID NO: 406 - DNA, 21 bp, Artificial Sequence
  Description of Artificial Sequence Synthetic oligonucleotide
gcguagaagagaccaaagutt

SEQ ID NO: 407 - DNA, 21 bp, Artificial Sequence
  Description of Artificial Sequence Synthetic oligonucleotide
cccucucaaccucaguauutt

SEQ ID NO: 408 - DNA, 21 bp, Artificial Sequence
  Description of Artificial Sequence Synthetic oligonucleotide
aauacugagguugagagggtt

SEQ ID NO: 409 - DNA, 21 bp, Artificial Sequence
  Description of Artificial Sequence Synthetic oligonucleotide
ccaaacacuuuggucucuutt

SEQ ID NO: 410 - DNA, 21 bp, Artificial Sequence
  Description of Artificial Sequence Synthetic oligonucleotide
aagagaccaaaguguuuggtt

SEQ ID NO: 411 - DNA, 21 bp, Artificial Sequence
  Description of Artificial Sequence Synthetic oligonucleotide
aaccucaguauuuucuauatt

SEQ ID NO: 412 - DNA, 21 bp, Artificial Sequence
  Description of Artificial Sequence Synthetic oligonucleotide
uauagaaaauacugagguutt

SEQ ID NO: 413 - DNA, 21 bp, Artificial Sequence
  Description of Artificial Sequence Synthetic oligonucleotide
gucuaucaguccaugaccatt

SEQ ID NO: 414 - DNA, 21 bp, Artificial Sequence
  Description of Artificial Sequence Synthetic oligonucleotide
uggucauggacugauagactt

SEQ ID NO: 415 - DNA, 21 bp, Artificial Sequence
  Description of Artificial Sequence Synthetic oligonucleotide
ucagcaccgagaacaucuatt

SEQ ID NO: 416 - DNA, 21 bp, Artificial Sequence
  Description of Artificial Sequence Synthetic oligonucleotide
uagauguucucggugcugatt

SEQ ID NO: 417 - DNA, 21 bp, Artificial Sequence
  Description of Artificial Sequence Synthetic oligonucleotide
ggugucuaucaguccaugatt

SEQ ID NO: 418 - DNA, 21 bp, Artificial Sequence
  Description of Artificial Sequence Synthetic oligonucleotide
ucauggacugauagacacctt

SEQ ID NO: 419 - DNA, 21 bp, Artificial Sequence
  Description of Artificial Sequence Synthetic oligonucleotide
ccccucucaaccucaguautt

SEQ ID NO: 420 - DNA, 21 bp, Artificial Sequence
  Description of Artificial Sequence Synthetic oligonucleotide
auacugagguugagaggggtt

SEQ ID NO: 421 - DNA, 21 bp, Artificial Sequence
  Description of Artificial Sequence Synthetic oligonucleotide
agcaccgagaacaucuacutt

SEQ ID NO: 422 - DNA, 21 bp, Artificial Sequence
  Description of Artificial Sequence Synthetic oligonucleotide
aguagauguucucggugcutt

SEQ ID NO: 423 - DNA, 21 bp, Artificial Sequence
  Description of Artificial Sequence Synthetic oligonucleotide
cagcaccgagaacaucuactt

SEQ ID NO: 424 - DNA, 21 bp, Artificial Sequence
  Description of Artificial Sequence Synthetic oligonucleotide
guagauguucucggugcugtt

SEQ ID NO: 425 - DNA, 21 bp, Artificial Sequence
  Description of Artificial Sequence Synthetic oligonucleotide
auggugucuaucaguccautt

SEQ ID NO: 426 - DNA, 21 bp, Artificial Sequence
  Description of Artificial Sequence Synthetic oligonucleotide
auggacugauagacaccautt

SEQ ID NO: 427 - DNA, 21 bp, Artificial Sequence
  Description of Artificial Sequence Synthetic oligonucleotide
aacacuuuggucucuucuatt

SEQ ID NO: 428 - DNA, 21 bp, Artificial Sequence
  Description of Artificial Sequence Synthetic oligonucleotide
uagaagagaccaaaguguutt

SEQ ID NO: 429 - DNA, 21 bp, Artificial Sequence
  Description of Artificial Sequence Synthetic oligonucleotide
agagaccagaucccugucutt

SEQ ID NO: 430 - DNA, 21 bp, Artificial Sequence
  Description of Artificial Sequence Synthetic oligonucleotide
agacagggaucuggucucutt

SEQ ID NO: 431 - DNA, 21 bp, Artificial Sequence
  Description of Artificial Sequence Synthetic oligonucleotide
accaugucugcccuaauuatt

SEQ ID NO: 432 - DNA, 21 bp, Artificial Sequence
  Description of Artificial Sequence Synthetic oligonucleotide
uaauuagggcagacauggutt

SEQ ID NO: 433 - DNA, 21 bp, Artificial Sequence
  Description of Artificial Sequence Synthetic oligonucleotide
accucaguauuuucuauactt

SEQ ID NO: 434 - DNA, 21 bp, Artificial Sequence
  Description of Artificial Sequence Synthetic oligonucleotide
guauagaaaauacugaggutt

SEQ ID NO: 435 - DNA, 21 bp, Artificial Sequence
  Description of Artificial Sequence Synthetic oligonucleotide
cucucaaccucaguauuuutt

SEQ ID NO: 436 - DNA, 21 bp, Artificial Sequence
  Description of Artificial Sequence Synthetic oligonucleotide
aaaauacugagguugagagtt

SEQ ID NO: 437 - DNA, 21 bp, Artificial Sequence
  Description of Artificial Sequence Synthetic oligonucleotide
ugucuaucaguccaugacctt

SEQ ID NO: 438 - DNA, 21 bp, Artificial Sequence
  Description of Artificial Sequence Synthetic oligonucleotide
ggucauggacugauagacatt

SEQ ID NO: 439 - DNA, 21 bp, Artificial Sequence
  Description of Artificial Sequence Synthetic oligonucleotide
cauccggaccaagauguuctt

SEQ ID NO: 440 - DNA, 21 bp, Artificial Sequence
  Description of Artificial Sequence Synthetic oligonucleotide
gaacaucuugguccggaugtt

SEQ ID NO: 441 - DNA, 21 bp, Artificial Sequence
  Description of Artificial Sequence Synthetic oligonucleotide
ucucaaccucaguauuuuctt

SEQ ID NO: 442 - DNA, 21 bp, Artificial Sequence
  Description of Artificial Sequence Synthetic oligonucleotide
gaaaauacugagguugagatt

SEQ ID NO: 443 - DNA, 21 bp, Artificial Sequence
  Description of Artificial Sequence Synthetic oligonucleotide
ucauccggaccaagauguutt

SEQ ID NO: 444 - DNA, 21 bp, Artificial Sequence
  Description of Artificial Sequence Synthetic oligonucleotide
aacaucuugguccggaugatt

SEQ ID NO: 445 - DNA, 21 bp, Artificial Sequence
  Description of Artificial Sequence Synthetic oligonucleotide
aauggugucuaucaguccatt

SEQ ID NO: 446 - DNA, 21 bp, Artificial Sequence
  Description of Artificial Sequence Synthetic oligonucleotide
uggacugauagacaccauutt

SEQ ID NO: 447 - DNA, 21 bp, Artificial Sequence
  Description of Artificial Sequence Synthetic oligonucleotide
ccucaguauuuucuauacatt

SEQ ID NO: 448 - DNA, 21 bp, Artificial Sequence
  Description of Artificial Sequence Synthetic oligonucleotide
uguauagaaaauacugaggtt

SEQ ID NO: 449 - DNA, 21 bp, Artificial Sequence
  Description of Artificial Sequence Synthetic oligonucleotide
acuuuggucucuucuacgctt

SEQ ID NO: 450 - DNA, 21 bp, Artificial Sequence
  Description of Artificial Sequence Synthetic oligonucleotide
gcguagaagagaccaaagutt

SEQ ID NO: 451 - DNA, 21 bp, Artificial Sequence
  Description of Artificial Sequence Synthetic oligonucleotide
cccucucaaccucaguauutt

SEQ ID NO: 452 - DNA, 21 bp, Artificial Sequence
  Description of Artificial Sequence Synthetic oligonucleotide
aauacugagguugagagggtt

SEQ ID NO: 453 - DNA, 21 bp, Artificial Sequence
  Description of Artificial Sequence Synthetic oligonucleotide
ccaaacacuuuggucucuutt

SEQ ID NO: 454 - DNA, 21 bp, Artificial Sequence
  Description of Artificial Sequence Synthetic oligonucleotide
aagagaccaaaguguuuggtt

SEQ ID NO: 455 - DNA, 21 bp, Artificial Sequence
  Description of Artificial Sequence Synthetic oligonucleotide
aaccucaguauuuucuauatt

SEQ ID NO: 456 - DNA, 21 bp, Artificial Sequence
  Description of Artificial Sequence Synthetic oligonucleotide
uauagaaaauacugagguutt

SEQ ID NO: 457 - DNA, 21 bp, Artificial Sequence
  Description of Artificial Sequence Synthetic oligonucleotide
gucuaucaguccaugaccatt

SEQ ID NO: 458 - DNA, 21 bp, Artificial Sequence
  Description of Artificial Sequence Synthetic oligonucleotide
uggucauggacugauagactt

SEQ ID NO: 459 - DNA, 21 bp, Artificial Sequence
  Description of Artificial Sequence Synthetic oligonucleotide
ucagcaccgagaacaucuatt

SEQ ID NO: 460 - DNA, 21 bp, Artificial Sequence
  Description of Artificial Sequence Synthetic oligonucleotide
uagauguucucggugcugatt

SEQ ID NO: 461 - DNA, 21 bp, Artificial Sequence
  Description of Artificial Sequence Synthetic oligonucleotide
ggugucuaucaguccaugatt

SEQ ID NO: 462 - DNA, 21 bp, Artificial Sequence
  Description of Artificial Sequence Synthetic oligonucleotide
ucauggacugauagacacctt

SEQ ID NO: 463 - DNA, 21 bp, Artificial Sequence
  Description of Artificial Sequence Synthetic oligonucleotide
ccccucucaaccucaguautt

SEQ ID NO: 464 - DNA, 21 bp, Artificial Sequence
  Description of Artificial Sequence Synthetic oligonucleotide
auacugagguugagaggggtt

SEQ ID NO: 465 - DNA, 21 bp, Artificial Sequence
  Description of Artificial Sequence Synthetic oligonucleotide
agcaccgagaacaucuacutt

SEQ ID NO: 466 - DNA, 21 bp, Artificial Sequence
  Description of Artificial Sequence Synthetic oligonucleotide
aguagauguucucggugcutt

SEQ ID NO: 467 - DNA, 21 bp, Artificial Sequence
  Description of Artificial Sequence Synthetic oligonucleotide
cagcaccgagaacaucuactt

SEQ ID NO: 468 - DNA, 21 bp, Artificial Sequence
  Description of Artificial Sequence Synthetic oligonucleotide
guagauguucucggugcugtt

SEQ ID NO: 469 - DNA, 21 bp, Artificial Sequence
  Description of Artificial Sequence Synthetic oligonucleotide
auggugucuaucaguccautt

SEQ ID NO: 470 - DNA, 21 bp, Artificial Sequence
  Description of Artificial Sequence Synthetic oligonucleotide
auggacugauagacaccautt

SEQ ID NO: 471 - DNA, 21 bp, Artificial Sequence
  Description of Artificial Sequence Synthetic oligonucleotide
aacacuuuggucucuucuatt

SEQ ID NO: 472 - DNA, 21 bp, Artificial Sequence
  Description of Artificial Sequence Synthetic oligonucleotide
uagaagagaccaaaguguutt

SEQ ID NO: 473 - DNA, 21 bp, Artificial Sequence
  Description of Artificial Sequence Synthetic oligonucleotide
agagaccagaucccugucutt

SEQ ID NO: 474 - DNA, 21 bp, Artificial Sequence
  Description of Artificial Sequence Synthetic oligonucleotide
agacagggaucuggucucutt